(19) United States
(12) Patent Application Publication
(10) Pub. No.: US 2026/0284258 A1
(43) Pub. Date: Sep. 24, 2026

(54) BIOGEL, COMPOSITIONS AND METHODS OF USE THE SAME

(71) Applicants: Rutgers, The State University of New Jersey, New Brunswick, NJ (US); Sung Kwang Medical Foundation, Seoul-Si (KR)

(72) Inventors: Jeffrey Luo, Manalapan, NJ (US); Ki-Bum Lee, Monmouth Junction, NJ (US); Inbo Han, Jeju-Do (KR)

(21) Appl. No.: 19/472,561

(22) PCT Filed: Dec. 11, 2023
(86) PCT No.: PCT/US2023/083411

§ 371 Date: Oct. 3, 2025

Related U.S. Application Data
(60) Provisional application No. 63/494,314, filed on Apr. 5, 2023.

(51) Int. Cl. A61L 27/22; A61L 27/10; A61L 27/26; A61L 27/38; A61L 27/52; A61L 27/54

(52) U.S. Cl. CPC A61L 27/222; A61L 27/10; A61L 27/26; A61L 27/3834; A61L 27/52; A61L 27/54; A61L 2300/414; A61L 2430/34; A61L 2430/38

(57) ABSTRACT

Provided herein, inter alia, are hydrogel compositions, and use thereof for treatment of a tissue in a subject and monitoring the treatment.

### CROSS-REFERENCE TO RELATED APPLICATIONS

This application claims the benefit under 35 U.S.C. § 119(e) of the earlier filing date of U.S. Provisional Patent No. 63/494,314 filed on Apr. 5, 2023, which is hereby incorporated by reference in its entirety.

### STATEMENT REGARDING FEDERALLY FUNDED RESEARCH

This invention was made with government support under Grant No. CBET-1803517 awarded by The National Science Foundation and Grant Nos. 1R-1DC016612, 3R01DC016612-01S1 and 5R01DC016612-02S1 awarded by The National Institutes of Health. The government has certain rights in the invention.

### REFERENCE TO AN ELECTRONIC SEQUENCE LISTING

The contents of the electronic sequence listing (070439_01801_SeqList.xml; Size: 26,700 bytes; and Date of Creation: Dec. 11, 2023) is herein incorporated by reference in its entirety.

### FIELD

Provided herein, inter alia, are soft tissue devices including hydrogel composites, compositions, and use for treating a tissue injury.

### BACKGROUND

Intervertebral disc (IVD) degeneration is a leading cause of back pain and precursor to more severe conditions, including disc herniation and spinal stenosis. While traditional growth factor therapies (e.g., TGF-3) are effective at transiently reversing degenerated disc by stimulation of matrix synthesis, it is increasingly accepted that bioscaffolds are required for sustained, complete IVD regeneration. Current scaffolds (e.g., metal/polymer composites, non-mammalian biopolymers) can be improved in one or more IVD regeneration demands: biodegradability, noninvasive injection, recapitulated healthy IVD biomechanics, predictable crosslinking, and matrix repair induction.

### SUMMARY

Provided herein, inter alia, are biogels and compositions or kits including the biogels. The biogels may include (i) a gelatine-based polymer that is formed by crosslinking two or more succinylated gelatine components, e.g., succinylated gelatine-tetrazine (“GelTZ”) and succinylated gelatin-norbornene (“GelNb”) and (ii) a biologically active material that can promote regeneration of tissues (e.g., damaged or injured tissues).

In an aspect, the disclosure provides a biogel including a gelatin polymer formed by crosslinking of a first gelatin precursor and a second gelatin precursor and a biologically active material. The first gelatin precursor includes one or more tetrazine groups and the second gelatin precursor includes one or more olefin groups.

In certain embodiments, the first gelatin precursor has a structure of Formula (I):

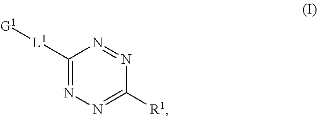

wherein:
G¹ is a gelatin protein, or a derivative or fragment thereof;
L¹ is a linking group selected from a bond, substituted or unsubstituted alkylene, or substituted or unsubstituted heteroalkylene; and
R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, and

the second gelatin precursor has a structure of Formula (II):

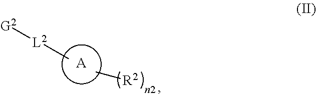

wherein:
G² is a gelatin protein, or a derivative or fragment thereof;
L² is a linking group selected from a bond, substituted or unsubstituted alkylene, or substituted or unsubstituted heteroalkylene;
Ring A is cycloalkenyl dienophile;
Each R² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein two or more R² may be same or different; or two or more R² may join to form a substituted or unsubstituted cycloalkyl, substituted or unsubstituted heterocycloalkyl, substituted or unsubstituted aryl, or substituted or unsubstituted heteroaryl; and
n2 is an integer from 0 to 6,

wherein G¹ and G² may be same or different.

In an aspect, the disclosure provides a method of treating a tissue injury in a subject comprising administering a biogel composition as described herein to the subject at the site of the tissue injury. In some embodiments, the subject has been or is suffering from an injury to a neural tissue, a cartilage, a muscle, or a bone. In some embodiments, the subject has been or is suffering from an injury to a neural tissue. In some embodiments, the subject has been or is suffering from degenerated intervertebral discs (IVD) and the biogel is applied to the site of IVD degeneration.

In some embodiments, the biogel is administered to the subject via exosomal delivery. In some embodiments, the subject suffers from traumatic brain injury (TBI). In some embodiments, the subject has been or is treated with neurosurgery.

The disclosure also provides a kit for treating a tissue in a subject. The kit includes a first vial comprising a first gelatin precursor; a second vial comprising a second gelatin precursor; a biologically active material; and an applicator.

Further provided are biocompatible tissue implants or tissue regenerating products including the biogel as described herein. In some embodiments, provided is an exosome comprising the biogel as described herein.

Other aspects of the invention are disclosed infra.

Where applicable or not specifically disclaimed, any one of the embodiments described herein are contemplated to be able to combine with any other one or more embodiments, even though the embodiments are described under different aspects of the invention.

### BRIEF DESCRIPTION OF THE DRAWINGS

FIG. 1 shows an exemplary injectable bioorthogonal hydrogel for intervertebral disc (IVD) regeneration. Gelatin-tetrazine (GelTz) and gelatin-norbornene (GelNb) are well-suited to form the basis for an injectable bioorthogonal hydrogel (BIOGEL). The bioorthogonal tetrazine and norbornene groups allow the liquid GelTz and GelNb precursors to form robust hydrogels approximately 5 minutes after simple mixing. Since the reaction is bioorthogonal (i.e., occurs under physiological conditions and does not react with endogenous biomolecules), this treatment can be combined with various therapeutic payloads and directly injected into patients to form minimally invasive hydrogels in situ. The resulting hydrogel is highly porous from the inert nitrogen gas byproduct, mimics native extracellular matrix (e.g., biocompatible, cell permissive), and mimics healthy IVD biomechanical properties.

FIG. 2A shows that precursor solutions were monitored for shear viscosity (typical room temperatures highlighted with a solid grey rectangle) at 1 Hz and unmodified gelatin increased in viscosity at room temperature, reducing ease of injection and necessitating more force for injection. In contrast, GelTz and GelNb displayed low viscosity across a wide temperature range, indicating high injectability regardless of environmental conditions and greater preservation of shear stress-sensitive payloads such as labile growth factors.

FIG. 2B shows that hydrogel crosslinking was monitored at 37° C. to simulate injection into a living patient. Precursor solutions do not exhibit any shear modulus increase, indicating no crosslinking and only mixed GelTz-GelNb solutions experienced increased shear modulus and cross-linking. Gelatin initiation (sustained G′ increase) began approximately 5 minutes post-mixing with a plateau at approximately 95 minutes.

FIG. 2C shows that in vitro culture of human primary IVD cells revealed that the precursor solutions are biocompatible. Moreover, cells undergo enhanced proliferation when seeded onto crosslinked BIOGEL substrates compared to PLL-coated tissue culture plastic control and Matrigel.

FIG. 2D shows that the biogel retained biodegradability via collagenase digestion. Crosslinked BIOGEL samples were subjected to varying concentrations of collagenase (type III) to measure biodegradation behavior. Hydrogel samples exhibited dose-dependent degradation, indicating the BIOGEL matrix can be remodeled and replaced as native cells infiltrate the BIOGEL scaffold. (n=3 biological replicates).

FIG. 2E shows that the biogel and its precursors were biocompatible with human induced pluripotent stem cell (hiPSC)-derived neuronal progenitor cells (NPC). Top: In vitro culture of human induced pluripotent stem cell-derived neuronal progenitor cells (hiPSC-NPC) revealed that BIOGEL precursor solutions are biocompatible. Moreover, cells underwent enhanced proliferation when seeded onto BIOGEL compared to the control wells. Bottom: BIOGEL promoted the formation of hiPSC-NPC cellular extensions whereas cells seeded on wells with undiluted and diluted Matrigel remained rounded. Relative viability was analyzed using one-way ANOVA followed by Dunnett's Test [α=0.05; * and ** indicate a statistically significant difference against all other conditions with different symbols, n=5 biological replicates].

FIG. 2F shows the Young's modulus of the biogel determined by determine atomic force microscopy.

FIG. 2G shows shear modulus and phase angle of the biogel determined by rheometry.

FIG. 2H shows a table summarizing some mechanical properties. The biogel exhibited mechanical properties similar to that of native IVD tissue reported in the literature.

FIG. 2I shows that the biogel retained its original mechanical properties even under significant strain (at least 10%), indicating high wear resistance and resiliency under mechanical load. i) Growth factors and other payloads (e.g., fluorescently labeled insulin) can be incorporated into the hydrogel by mixing with either precursor solution. Once the hydrogel was crosslinked, encapsulated payloads were released in a biphasic manner: an initial burst release followed by sustained release over time. This can be roughly modeled with an exponential decay function. Approximately 40% of the original loading mass remained within the hydrogel even after prolonged incubation. Relative viability was analyzed using one-way ANOVA followed by Dunnett's Test within cell groups [α=0.05; † and ‡ indicate a statistically significant difference against all other conditions with different symbols, n=5 biological replicates].

FIG. 2J shows that growth factors and other payloads (e.g., fluorescently labeled insulin) can be incorporated into the hydrogel by mixing with either precursor solution. Once the hydrogel is crosslinked, encapsulated payloads were released in a biphasic manner: an initial burst release followed by sustained release over time. This can be roughly modeled with an exponential decay function. Approximately 40% of the original loading mass remained within the hydrogel even after prolonged incubation. Relative viability was analyzed using one-way ANOVA followed by Dunnett's Test within cell groups [α=0.05; † and ‡ indicate a statistically significant difference against all other conditions with different symbols, n=5 biological replicates].

FIGS. 2K and 2L show biphasic drug release behavior persists in varied physiological conditions. FITC-insulin loaded biogel samples were incubated at varying pH and temperatures for comparison against typical physiological conditions. FIG. 2K shows that severe alkalosis (pH 8) did not include significant changes in drug release behavior compared to normal pH (7.2), while acidosis (pH 6) increased burst release. While the quantity of FITC-insulin increased during the burst release period, samples incubated at pH 6 still displayed sustained (reservoir) release after three days, similar to normal pH of 7.2. After two weeks, the difference between pH 6 and pH 7.2 was no longer statistically significant. FIG. 2L shows that incubating the biogel at 4° C. (hypothermia) initially resulted in significantly lower burst release compared to incubation at 37° C. (normal physiological temperature), but this difference subsided after one day. Differences in sustained release only became statistically significant after prolonged incubation (four weeks). Incubation at 43° C. (hyperthermia) failed to produce significant differences in FITC-insulin release until the sustained release period, where a small increase in drug release can be observed compared to 37° C. Drug release was analyzed using two-way repeated measurement ANOVA followed by Dunnett's Test (α=0.05, symbol indicates a statistically significant difference against pH 7.2 or 37° C., n=3 biological replicates).

FIG. 3A shows that after LPS exposure, both hAFCs and hNPCs experienced significantly increased expression of pro-inflammatory cytokines (CXCL1, IL-6, and MMP13). While TGF-β and BIOGEL led to statistically significant decreases in some genes, only TGF-β-BIOGEL led to consistent decreases in all three pro-inflammatory genes, indicating encapsulation of TGF-β within BIOGEL potentiates its anti-inflammatory effect.

FIG. 3B shows that while LPS was insufficient at upregulating Substance P in this hiPSC-NPC cell line, TGF-β BIOGEL was found to be effective at mitigating pain-related markers (CGRP and Substance P). RT-qPCR was analyzed using one-way ANOVA followed by Tukey's HSD [α=0.05, * indicates a statistically significant difference; †, ‡, and ~ indicates a statistically significant difference against all other conditions with different symbols, n=3 biological replicates].

FIGS. 4A, 4B and 4C show that fixed IVD sections were stained with hematoxylin & eosin (FIG. 4A) and Safranin O (FIG. 4B) (fast green counterstain) along with immunohistochemistry staining for Aggrecan (FIG. 4C) and Collagen type 2 (FIG. 4D). Staining area for (i) hematoxylin, (ii) Safranin O, (iii) Aggrecan, and (iv) Collagen type 2 was normalized to healthy, uninjured IVD. Out of all treatment conditions, only TGF-β loaded BIOGEL resulted in statistically significant increased staining across all targets. Additionally, TGF-β loaded BIOGEL led to the greatest recovery of staining among treatment conditions. FIG. 4D shows that holistic quantification of IVD injury/degeneration indicated TGF-β loaded biogel induced the greatest histological recovery. Staining and histological grading were analyzed using one-way ANOVA followed by Tukey's HSD [α=0.05, * indicates a statistically significant difference, † and ‡ indicates a statistically significant difference against all other conditions with different symbols, treatments: n=8 biological replicates, healthy and injury: n=24 biological replicates (i.e., 8 from each treatment group)]

FIGS. 4F, 4G and 4H show quantification of macrophage markers against total DAPI-positive cells. It is shown that TGF-β loaded BIOGEL promotes significantly less macrophage infiltration (M1, M2, and total) compared to all other conditions. CD86 (FIG. 4F)/CD163 (FIG. 4G)/Iba1 (FIG. 4H) % positivity was analyzed using one-way ANOVA followed by Tukey's HSD [α=0.05, symbol indicates a statistically significant difference against conditions with different symbols, treatments: n=4 biological replicates, healthy and injury: n=12 biological replicates (i.e., 4 from each treatment group)].

FIG. 5A shows schematic diagram of in vivo IVD degeneration model. Nucleotomies were performed on the Co4-5 and Co5-6 IVD of Sprague-Dawley rats. Immediately after nucleotomies, treatments consisting of TGF-β solution, BIOGEL, and TGF-β loaded BIOGEL were injected into the Co4-5 nucleus pulposus void space. Co3-4 IVD were monitored and used as a healthy control condition.

FIG. 5B shows healthy IVD was defined by well-hydrated tissue (bright signal under T2-MRI) and lack of painful sensations under benign conditions (allodynia).

FIG. 5C shows that grading for IVD MRI index revealed that treatment with bare biogel and TGF-β loaded BIOGEL resulted in significant recovery.

FIG. 5D shows that quantification of CGRP staining against DAPI staining (cell nucleus) revealed that TGF-β loaded biogel induced significantly reduced pain compared to non-treated injuries and bare biogel.

FIG. 5E shows that allodynia was assessed by measuring the amount of force (applied via Von Frey fibers) required to induce paw withdrawal in 50% of testing measurements. Starting two days post-treatment, rats with TGF-β loaded biogel hydrogels showed a greater withdrawal threshold compared to rats treated with TGF-β solution and bare biogel, indicating greater recovery from allodynia. MRI index, relative CFRP intensity per DAPI, and 50% withdrawal threshold were analyzed using one-way ANOVA followed by Tukey's HSD [α=0.05; * indicates a statistically significant difference, indicates a statistically significant difference against all other conditions with different symbols; treatments (CGRP and MRI): n=4 biological replicates, healthy and injury: n=12 biological replicates (i.e., 4 from each treatment group); treatments (Von Frey): n=6].

FIG. 6 shows an exemplary tetrazine methylamine synthesis. Schematic diagram showing key steps in the synthesis of 4-(1,2,4,5-tetrazin-3-yl)phenyl)methanamine hydrochloride (Tetrazine methylamine).

FIG. 7 shows a schematic diagram showing the chemical structure and synthesis steps of the injectable bioorthogonal hydrogel system.

FIG. 8A shows that gelTz exhibited a peak at 518 nm (resulting in a vibrant red color) due to the highly conjugated structure of tetrazine methylamine. The other precursors lacked such absorption peaks or notable coloration.

FIG. 8B shows that hydrogel crosslinking was non-invasively tracked by monitoring for color change as the tetrazine conjugated π-π structure is destroyed during reaction with norbornene, leading to a pale yellow-orange hydrogel and the tetrazine-based peak at 518 nm rapidly decreased over the course of the first 5 minutes, leading to a loss of the distinct red color normally found with (unreacted) GelTz.

FIG. 8C shows that the product hydrogel displayed near 100% degree of crosslinking as evidenced by the complete disappearance of the Tetrazine Methylamine absorbance peak after 100 minutes post-mixing.

FIG. 9A shows that MnO₂ nanosheets dispersed in BIOGEL precursor solutions to form MnO2-loaded BIOGEL. Since BIOGEL relies on bioorthogonal tetrazine-norbornene ligation, MnO₂ exhibits minimal interference with the crosslinking reaction.

FIG. 9B shows that MnO₂ can modulate drug release (triggered or sustained release). (i) When free fluorescein and fluorescein-MnO2 complexes were loaded into BIOGEL in phosphate buffered saline (PBS), fluorescein-MnO2 BIOGEL exhibited reduced burst release compared to free fluorescein. Additionally, both forms of fluorescein exhibited sustained release after approximately 3 days. (ii) When fluorescein-MnO2 BIOGEL was immersed in glutathione (GSH)-containing PBS, significantly more fluorescein was released from the degraded MnO2 complex. Moreover, the enhanced fluorescein release is dose dependent. The addition of more glutathione (at week 2) resulted in additional fluorescein release. (iii) These drug release curves indicate MnO2-BIOGEL reduced burst release and interacted with bioreductants (e.g., GSH) to accelerate drug release.

FIG. 9C shows that the introduction of MnO2 into the BIOGEL precursor solutions did not appreciably alter crosslinking kinetics (i.e., crosslinking initiation at 5 minutes, plateau at 90 minutes); however, the resulting hydrogel was significantly softer. This reduction in shear modulus is advantageous for SCI regeneration since neurogenesis is promoted on softer substrates.

FIG. 9D shows that despite the softer hydrogel, MnO2-BIOGEL remains intact (i.e., no yielding or mechanical failure) under the same amount of strain as bare BIOGEL.

FIG. 9E shows that bare BIOGEL and MnO₂—BIOGEL hydrogels exhibited different shear moduli (storage and loss) in response to varying measurement frequency, which indicates viscoelasticity.

FIG. 9F shows that the viscoelastic behavior of BIOGEL (quantified as phase angle) was found to be similar regardless of whether hydrogels contained MnO2. The monotonically increasing phase angle indicates the bare BIOGEL and MnO2-BIOGEL exhibited viscoelastic solid behavior.

FIG. 9G shows that the inclusion of MnO2 in the BIOGEL precursor solution (specifically gelatin-tetrazine solution) does not appreciably alter shear viscosity (i.e., how easily injectable the solution is). Fluorescein drug release was analyzed using a three-way ANOVA with Tukey's HSD post-hoc testing. Hydrogel elastic modulus was analyzed using Student's t-test [α=0.05; * indicates a statistically significant difference, n=3 biological replicates].

FIG. 10A shows that DAPT-MnO₂ BIOGEL induces neurogenesis and halts inflammation in an in vitro SCI Model. A cell culture insert-based, in vitro SCI model was developed to verify the ability for BIOGEL-based therapies to induce neurogenesis and halt inflammatory pathways. Human macrophages were seeded on the surface of the well and induced to a pro-inflammatory phenotype via chronic lipopolysaccharide (LPS) exposure. Meanwhile, human induced pluripotent stem cell-derived neuronal progenitor cells (hiPSC-NPC) were encapsulated in BIOGEL supplemented with DAPT and/or MnO2. These cell-loaded hydrogels were placed in a cell culture insert, which allowed pro-inflammatory cytokines to diffuse from seeded macrophages to encapsulated hiPSC-NPC.

FIG. 10B shows that these immunocytochemistry staining images were quantified with ImageJ to verify that DAPT-MnO2 promoted the greatest number of cells with Tuj1-positive, neuron-like morphologies.

FIG. 10C shows that macrophages exposed to LPS expressed significantly more TNFα, IL-6, and MMP2 compared to their non-LPS controls. Co-culturing BIOGEL with MnO2 and DAPT resulted in modest reduction, but co-culture with DAPT-MnO2 resulted in the most consistent downregulation of these deleterious, pro-inflammatory cytokines. Staining and RT-qPCR were analyzed using one-way ANOVA followed by Tukey's HSD (α=0.05, * indicates a statistically significant difference, † and ‡ indicates a statistically significant difference against all other conditions with different symbols, n=3 biological replicates).

FIG. 11A shows that neurogenesis [induced by Notch inhibition via DAPT release] was defined by fewer neural stem/neuronal progenitor cells and greater mature neuronal markers. Here, DAPT-MnO2 BIOGEL treatments inhibited Notch3 and Sox2 expression. Although the Nestin and MAP2 expression difference between bare and DAPT-MnO2 was not statistically significant, the pro-neurogenic expression changes nonetheless warranted further interest.

FIG. 11B shows that key SCI microenvironment parameters of interest included reactive gliosis, pro-/anti-inflammatory cytokines, and cell proliferation. DAPT-MnO2 BIOGEL treatments were significantly more effective at downregulating GFAP and IL-1β expression while upregulating IL-10 and Ki67. Gene expression was analyzed using Students' T Test [α=0.05; * indicates a statistically significant difference; n=3].

FIGS. 12A, 12B, and 12C are schematics of using the BIOGEL to treat Traumatic Brain Injury (TBI). FIG. 12A is schematic of TBI. FIG. 12B illustrates exosomes that were prepared via ex vivo engineering of iPSC-NPCs with deferoxamine (DFO) to mimic hypoxic conditions. FIG. 12C depicts how the BIOGEL overcomes challenges in TBI treatment.

FIGS. 13A, 13B, 13C, 13D, and 13E show isolation and characterization of exosomes with BIOGEL. FIG. 13A is a schematic of the co-encapsulation of exosomes and neural progenitor cells (NPCs) inside the BIOGEL. FIG. 13B is a schematic of DFO-conditioned exosomes. FIG. 13C is a graph that shows nanoparticle tracking analysis. FIG. 13D is a graph that shows the zeta potential and intensity of the exosomes. FIG. 13E is a Western blot depicting the exosome marker CD81 and actin as a loading control.

FIGS. 14A, 14B, 14C, 14D, 14E, and 14F show the results of brain samples from a controlled cortical impact (CCI) model of brain injury that was implemented by inducing trauma with an impactor between the bregma and lambda points on the rat brain. FIG. 14A shows the surgery workflow. FIG. 14B shows the experimental timeline. FIG. 14C is a schematic of the axes of the brain. FIG. 14D shows images of the cortex and hippocampus. FIG. 14E is a graph of the lesion cavity area of the indicated conditions. FIG. 14F show H&E images of the brain samples.

FIGS. 15A, 15B, 15C, 15D, 15E, 15F, 15G, and 15H show that DFO-treated exosome-loaded BIOGEL reduces brain scarring and increases BDNF levels. FIG. 15A is a schematic of neurotrophin release. FIG. 15B shows graphs of the relative ratio of CS56 or p75NTR area of the indicated conditions. FIG. 15C is a graph of the relative ratio of BDNF area of the indicated conditions. FIG. 15D is a graph of the concentration of BDNF of the indicated conditions. FIG. 15E shows graphs of the relative ratio of MAP2 or Syn1 area of the indicated conditions. FIG. 15F shows graphs of the relative ratio of SOX2 or Ki67 of the indicated conditions. FIG. 15G is a schematic of the indicated cells. FIG. 15H is a graph of the activation cells in the subgranular zone (SGZ) for the indicated conditions. NS=not significant.

FIGS. 16A, 16B, 16C, 16D, 16E, and 16F illustrate that BIOGEL with DFO-conditioned exosomes protects oligodendrocytes in vivo. FIG. 16A is a schematic of the protocol. FIG. 16B shows images of the brain with Luxol Fast Blue staining. FIG. 16C is a graph of the remyelination area/total area of the indicated conditions. FIG. 16D is a graph of the relative ratio of Sox10 area of the indicated conditions. FIG. 16E is a graph of the relative ratio of TUJ1 area of the indicated conditions. FIG. 16F is a graph of the relative ratio of NeuN area of the indicated conditions. FIG. 16G is a graph of the relative ratio of MBP area of the indicated conditions. NS=not significant.

FIGS. 17A, 17B, 17C, 17D, 17E, and 17F show that DFO-treated exosome-loaded BIOGEL improves angiogenesis, repairs Blood-Brain Barrier, reduces inflammation, and boosts functional recovery. FIG. 17A is a graph of the relative ratio of CD31 area of the indicated conditions. FIG. 17B shows graphs of the relative ratio of PDGFRβ or occluding area of the indicated conditions. FIG. 17C shows graphs of the relative ratio of CD86, CD163, or lba1 area of the indicated conditions. FIG. 17D is a graph of the results of a rotarod test in rates treated with the indicated conditions. FIG. 17E is a graph of the modified neurological severity score (mNSS) in rats exposed to the indicated conditions (FIG. 17D). FIG. 17F is a schematic of the rotarod performance test.

### DETAILED DESCRIPTION

Provided herein are nano-hybrid peptide hydrogels (“hydrogel composite”) that deliver pro-regenerative cytokines, suppress inflammatory factors, and construct a regenerative ECM microenvironment after IVD degeneration induction. The transplantation of this nanomaterial-based therapeutic hydrogels into a nucleotomy model increases ECM protein expression, facilitates nucleus pulposus cell differentiation, accelerates tissue regeneration, and further decreases pain associated with IVD degeneration. The nanomaterial-based hydrogels disclosed herein are highly biocompatible, completely biodegradable, and injectable. As a result, the nano-hybrid peptide hydrogels as disclosed herein provide new therapeutic strategies for the treatment of IVD degeneration as well as other degenerative diseases and injuries.

The following detailed description, given by way of example, but not intended to limit the invention solely to the specific embodiments described, may best be understood in conjunction with the accompanying drawings.

### Definitions

The abbreviations used herein have their conventional meaning within the chemical and biological arts. The chemical structures and formulae set forth herein are constructed according to the standard rules of chemical valency known in the chemical arts.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non-adjacent carbon atoms of the monocyclic ring are linked by an alkylene bridge of between one and three additional carbon atoms (i.e., a bridging group of the form (CH₂)_{w}, where w is 1, 2, or 3).

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle.

The term “alkylene,” by itself or as part of another substituent, means, unless otherwise stated, a divalent radical derived from an alkyl, as exemplified, but not limited by, —CH₂CH₂CH₂CH₂— Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., N, S, Si, or P) may be placed at any interior position of the heteroalkyl group or at the position at which the alkyl group is attached to the remainder of the molecule. Heteroalkyl is an uncyclized chain.

The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. A “cycloalkylene” and a “heterocycloalkylene,” alone or as part of another substituent, means a divalent radical derived from a cycloalkyl and heterocycloalkyl, respectively.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring).

The term “substituted” means to contain or include at least one “substituent group”. For example, the “substituent group,” as used herein, means a group selected from oxo, halogen, —CF₃, —CCl₃, —CBr₃, —CI₃, —CHF₂, —CHCl₂, —CHBr₂, —CHI₂, —CH₂F, —CH₂Cl, —CH₂Br, —CH₂I, —CN, —N₃, —OH, —NH₂, —COOH, —CONH₂, —NO₂, —SH, —SCH₃, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCF₃, —OCCl₃, —OCBr₃, —OCl₃, —OCHF₂, —OCHCl₂, —OCHBr₂, —OCHI₂, —OCH₂F, —OCH₂Cl, —OCH₂Br, —OCH₂I, unsubstituted alkyl (e.g., —C₁-C₈alkyl, —C₁-C₆alkyl, or C₁-C₄alkyl), unsubstituted heteroalkyl (e.g., —2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, as within a range of normal tolerance in the art, for example, within 2 standard deviations of the mean. In certain embodiments, “about” can be understood as within 10%, 9%, 8%, 7%, 6%, 5%, 4%, 3%, 2%, 1%, 0.5%, 0.1%, 0.05%, or 0.01% of the stated value.

When a range is described for a variable, it will be understood that the variable includes all values including the end points described within the stated range. For example, the range of “5 to 10” will be understood to include any subranges, such as 6 to 10, 7 to 10, 6 to 9, 7 to 9, and the like, as well as individual values of 5, 6, 7, 8, 9 and 10, and will also be understood to include any value between valid integers within the stated range, such as 5.5, 6.5, 7.5, 5.5 to 8.5, 6.5 to 9, and the like. Also, for example, the range of “10% to 30%” will be understood to include subranges, such as 10% to 15%, 12% to 18%, 20% to 30%, etc., as well as all integers including values of 10%, 11%, 12%, 13% and the like up to 30%, and will also be understood to include any value between valid integers within the stated range, such as 10.5%, 15.5%, 25.5%, and the like.

As used herein the specification, “patient,” “subject” or “subjects” or “individuals” may be used interchangeably and include, but are not limited to, mammals such as humans or non-human mammals, e.g., domesticated, agricultural or wild, animals, as well as birds, and aquatic animals. In certain embodiments, the subject is a human patient, anon-human patient or an animal subjected to medical treatment.

As used herein, the term “gel” refers to a solid three-dimensional network that spans the volume of a liquid medium and ensnares it through surface tension effects. This internal network structure may result from physical bonds (physical gels) or chemical bonds (chemical gels), as well as crystallites or other junctions that remain intact within the extending fluid. Virtually any fluid can be used as an extender including water (hydrogels), oil, and air (aerogel). Both by weight and volume, gels are mostly fluid in composition and thus exhibit densities similar to those of their constituent liquids. A hydrogel is a type of gel that uses water as a liquid medium.

As used herein, the term “biogel” is a type of gel that is formed by bioorthogonal reaction, e.g., click chemistry or cross-linking, that does not interfere with surrounding molecules or biomolecules. The biorthogonal reaction may include, but is not limited to, a click chemistry and bioorthogonal tetrazine-norbornene crosslinking reaction.

As used herein, the term “biologically active material” refers to a material that naturally interacts or reacts with biological systems, or is engineered to interact or react with biological systems. In certain embodiments, the biologically active material may include any one of a protein, antibody, nucleic acid, biologic drug, peptide, small molecule, ligand, cytokine, chemotherapeutic agent, antipyretic, analgesic, anesthetic, antibiotic, antiseptic, hormone, stimulant, depressant, statin, beta blocker, anticoagulant, antiviral, anti-fungal, anti-inflammatory, growth factor, vaccine, diagnostic composition, psychiatric medication, and psychoactive compound, or combinations thereof. The biological system can include, for example, an individual cell, a set of cells (e.g., a cell culture), an organ, a tissue, or a multi-cellular organism.

As used herein, the term “antibody” (Ab) is used in the broadest sense, and specifically may include any immunoglobulin, whether natural, or partly, or wholly synthetically produced, including, but not limited to monoclonal antibodies, polyclonal antibodies, multispecific antibodies (for example, bispecific antibodies and polyreactive antibodies), and antibody fragments. Thus, the term “antibody,” as used in any context within this specification, is meant to include, but not be limited to, any specific binding member, immunoglobulin class and/or isotype (e.g., IgG1, IgG2a, IgG2b, IgG3, IgG4, IgM, IgA1, IgA2, IgD, and IgE), and biologically relevant fragment, or specific binding member thereof, including, but not limited to, Fab, F(ab′)₂, scFv (single chain or related entity) and (scFv)₂.

As used herein, the term “cytokine” may refer to any substances secreted by cells of the immune system that have an effect on other cells, including both anti-inflammatory and pro-inflammatory cytokines. Exemplary cytokines include, but are not limited to, those in the IL-1 superfamily, TNF superfamily, interferons, chemokines, and IL-6 superfamily, as well receptors of any cytokines.

As used herein, the term “nucleic acid,” may refer to a polymer composed of a multiplicity of nucleotide units (ribonucleotide, deoxyribonucleotide, or related structural variants) linked via phosphodiester bonds, including but not limited to, DNA or RNA. The term encompasses sequences that include any of the known base analogs of DNA and RNA. Examples of a nucleic acid include, and are not limited to, mRNA, miRNA, tRNA, rRNA, snRNA, siRNA, dsRNA, cDNA and DNA/RNA hybrids. Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil (U), adenine (A), thymine (T), cytosine (C), guanine (G), and their derivative compounds. Nucleic acids may be obtained by chemical synthesis methods or by recombinant methods. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

As used herein, the term “peptide” may refer to peptide compounds containing two or more amino acids linked by the carboxyl group of one amino acid to the amino group of another, to form an amino acid sequence. Peptides may be purified and/or isolated from natural sources or prepared by recombinant or synthetic methods.

As used herein, the term “small molecule” may refer to non-peptidic, non-oligomeric organic compounds, either synthesized or found in nature. These compounds may be “natural product-like,” however, the term “small molecule” is not limited to “natural product-like” compounds. Small molecules are typically characterized in that they possess one or more of the following characteristics: several carbon-carbon bonds, multiple stereocenters, multiple functional groups, at least two different types of functional groups, and a molecular weight of less than 1500 Dalton, although not all, or even multiple, of these features need to be present. In certain embodiments, the “small molecule” or the like preferably has a molecule having a molecular weight of less than about 700 Dalton, e.g., less than about 1,000, 900, 800, 700, 650, 600, 550, 500, 450, 400, 350, 300, 250, 200, 100, or even 50 Dalton.

The term “growth factor” as used herein refers to a class of protein or natural substance (e.g., hormone) that stimulates growth, proliferation, maturation, healing or differentiation of cells or tissues. Exemplary growth factors may include, but not limited to, growth/differentiation factor (GDF), epidermal growth factor (EGF), platelet-derived growth factor (PDGF), vascular endothelial growth factor (VEGF), fibroblast growth factor (FGF), transforming growth factor-alpha (TGF-α), transforming growth factor-beta (TGF-β), nerve growth factor (NGF), placental growth factor (PIGF), VEGF/PIGF heterodimers, and connective tissue growth factor (CTGF).

In certain embodiments, the growth factor may include TGF-β or its isoforms (e.g., UniProt ID: P01137 (human TGF-β1), P61812 (human TGF-β2), or P10600 (human TGF-β3)) which is multifunctional cytokine and its activated complexes with other component may form kinase complex which regulates many cellular functions including cell proliferation, differentiation, adhesion and migration. For instance, TGF-β increases survival or regenerations of neurones in the nervous system (e.g., central or peripheral nervous system, or IVD).

The term “polymer” includes linear and branched polymer structures, and also encompasses crosslinked polymers as well as copolymers (which may or may not be crosslinked), thus including block copolymers, alternating copolymers, random copolymers, and the like. The polymer may be naturally occurring or obtained from synthetic sources. In certain embodiments, the polymer may be added to the composite or composition thereof to implement supplemental, desired properties to the composite.

As used herein, the term “biodegradable” refers to a material that can be broken down by biological means in a subject.

As used herein, the term “implantable” means able to be formulated for implantation via a syringe to a subject.

As used herein, the term “soft tissue” refers to tissues that connect, support, or surround other structures and organs of the body. Soft tissue includes muscles, tendons, ligaments, fascia, nerves, fibrous tissues, fat, blood vessels, and synovial membranes.

As used herein, the term “neural tissue” and “nervous system” refers to a biological system involving neuron tissues or cells and can cover a central nervous system including brain and spinal cord, and the peripheral nervous system including the autonomic and somatic nervous systems.

As used herein, the term “stable” refers to a material that does not degrade at room temperature.

As used herein, the term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. In certain embodiments, the “treating” or “treatment” includes “preventing” or “prevention” of disease. In certain embodiments, the “treating” or “treatment” may not include “preventing” or “prevention” of disease. The terms “prevent” or “preventing” refer to prophylactic and/or preventative measures, wherein the object is to prevent, or slow down the targeted pathologic condition or disorder. In addition, “treating” or “treatment” does not require complete alleviation of signs or symptoms, does not require a cure, and specifically includes protocols that have only a marginal effect on the patient.

### Biogel Composition

In an aspect, a biogel includes (a) a gelatin polymer formed by crosslinking of a first gelatin precursor and a second gelatin precursor and (b) a biologically active material. The first gelatin precursor includes one or more tetrazine groups and the second gelatin precursor includes one or more olefin groups.

In certain aspects, the first gelatin precursor has a structure of Formula (I):

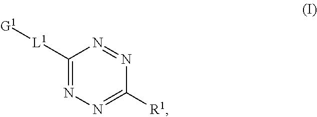

wherein:
G¹ is a gelatin protein, or a derivative or fragment thereof;
L¹ is a linking group selected from a bond, substituted or unsubstituted alkylene, or substituted or unsubstituted heteroalkylene; and
R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In certain aspects, the second gelatin precursor has a structure of Formula (II):

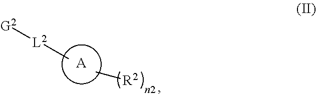

wherein:
G² is a gelatin protein, or a derivative or fragment thereof;
L² is a linking group selected from a bond, substituted or unsubstituted alkylene, or substituted or unsubstituted heteroalkylene;
Ring A is cycloalkenyl dienophile;
Each R² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein two or more R² may be same or different; or two or more R² may join to form a substituted or unsubstituted cycloalkyl, substituted or unsubstituted heterocycloalkyl, substituted or unsubstituted aryl, or substituted or unsubstituted heteroaryl; and
n2 is an integer from 0 to 6.

In some embodiments, the G¹ and G² are same. In some embodiments, the G¹ and G² are different.

In some embodiments, each L^{1A}, L^{1B}, L^{1C}, L^{2A}, L^{2B}, and L^{2C} is independently a bond, substituted or unsubstituted C₁-C₈ alkylene, or substituted or unsubstituted phenylene. In some embodiments, at least one of L^{1A}, L^{1B}, and L^{1C} is a bond. In some embodiments, at least one of L^{2A}, L^{2B}, and L^{2C} is a bond. In some embodiments, at least one of L^{1A}, L^{1B}, and L^{1C} is a substituted or unsubstituted C₁-C₈ alkylene. In some embodiments, at least one of L^{2A}, L^{2B}, and L^{2C} is a substituted or unsubstituted C₁-C₈ alkylene. In some embodiments, at least one of L^{1A}, L^{1B}, and L^{1C} is a substituted or unsubstituted phenylene. In some embodiments, at least one of L^{2A}, L^{2B} and L^{2C} is a substituted or unsubstituted phenylene.

In some embodiments, L¹ is —NH(CO)-L^{1A}-C(O)NH-L^{1B}-L^{1C}-, —NH(CO)-L^{1A}-C(O)-L^{1B}-L^{1C}-, or —NH(CO)-L^{1A}-NH-L^{1B}-L^{1C}-. In certain embodiments, L¹ is —NH(CO)-L^{1A}-C(O)NH-L^{1B}-L^{1C}-, and each L^{1A}, L^{1B}, and L^{1C} is independently a bond or substituted or unsubstituted C₁-C₄ alkylene. In certain embodiments, L¹ is —NH(CO)-L^{1A}-C(O)-L^{1B}-L^{1C}-, and each L^{1A}, L^{1B}, and L^{1C} is independently a bond or substituted or unsubstituted C₁-C₄ alkylene. In certain embodiments, L¹ is —NH(CO)-L^{1A}-NH-L^{1B}-L^{1C}- and each L^{1A}, L^{1B}, and L^{1C} is independently a bond or substituted or unsubstituted C₁-C₄ alkylene.

In some embodiments, L² is —NH(CO)-L^{2A}-C(O)NH-L^{2B}-L^{2C}-, —NH(CO)-L^{2A}-C(O)-L^{2B}-L^{2C}-, or —NH(CO)-L^{2A}-NH-L^{2B}-L^{1C}. In certain embodiments, L² is —NH(CO)-L^{2A}-C(O)NH-L^{2B}-L^{2C}- and each L^{1A}, L^{1B}, and L^{1C} is independently a bond, substituted or unsubstituted C₁-C₄ alkylene, or substituted or unsubstituted phenylene. In certain embodiments, L² is —NH(CO)-L^{2A}-C(O-L^{2B}-L^{2C}- and each L^{1A}, L^{1B}, and L^{1C} is independently a bond, substituted or unsubstituted C₁-C₄ alkylene, or substituted or unsubstituted phenylene. In certain embodiments, L² is —NH(CO)-L^{2A}-NH-L^{2B}-L^{2C}- and each L^{1A}, L^{1B}, and L^{1C} is independently a bond, substituted or unsubstituted C₁-C₄ alkylene, or substituted or unsubstituted phenylene.

In certain embodiments, each L¹ and L² is independently —NH(CO)—(CH₂)ₚ—C(O)NH—(CH₂)_{q}—, or —NH(CO)—(CH₂)ₚ—C(O)NH—(CH₂)_{q}—(C₆H₅)—. In certain embodiments, L¹ is —NH(CO)—(CH₂)ₚ—C(O)NH—(CH₂)_{q}—, and L² is —NH(CO)—(CH₂)ₚ—C(O)NH—(CH₂)_{q}—(C₆H₅)—.

In certain embodiments, R¹ is hydrogen. In certain embodiments, n2 is 0 or 1.

In one aspect, the biogel composition includes the gelatin polymer formed by the first gelatin precursor having a structure of

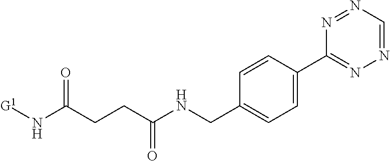

and the second gelatin precursor having a structure of

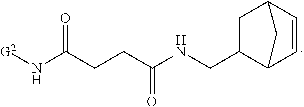

In certain embodiments, each G¹ and G² independently includes a gelatin protein derived from aggrecan or collagen.

The biologically active material comprises a protein, antibody, nucleic acid, biologic drug, peptide, small molecule, ligand, cytokine, chemotherapeutic agent, antipyretic, analgesic, anesthetic, antibiotic, antiseptic, hormone, stimulant, depressant, statin, beta blocker, anticoagulant, antiviral, anti-fungal, anti-inflammatory, growth factor, vaccine, cell, stem cell, tissue, diagnostic composition, psychiatric medication, psychoactive compound, or combinations thereof.

The biogel composition as described herein may be advantageously utilized by containing one or more biologically active materials when applied to a subject, for example, on a body surface (e.g., a site of tissue injury).

In certain embodiments, the composite may include one or more growth factors (e.g., exogenous growth factors) for growth, proliferation, maturation, healing or differentiation of cells or tissues. Exemplary growth factors may include, but not limited to, growth/differentiation factor (GDF), epidermal growth factor (EGF), platelet-derived growth factor (PDGF), vascular endothelial growth factor (VEGF), fibroblast growth factor (FGF), transforming growth factor-alpha (TGFα) and/or nerve growth factors (NGF's).

For example, the biogel including EGF/TGF may advantageously be used in the acceleration of wound healing and burns, reduction in keloid scar formation (especially for burns), skin engraftment dressings, and the treatment of chronic leg ulcers. The biogel including VEGF may promote angiogenesis (blood vessel growth) or contribute to angiogenesis both indirectly and directly by stimulating proliferation of endothelial cells at the microvessel level, causing them to migrate and to alter their generic expression. The biogel including FGF may promote or induce angiogenic in vivo and its angiogenicity is enhanced by combined use of TNF. Also, keratinocyte growth factor (KGF) may be suitably contained in the biogel for wound healing and other disorders involving epithelial cell destruction. Further, the biogel including transforming growth factors (TGF) may transform various cell lines, for example, may have the ability to grow in culture for more than a limited number of generations, growth in multiple layers rather than monolayers, and the acquisition of an abnormal karyotype. In certain embodiments, the biogel may include one or more growth factors (e.g., exogenous growth factors) alone or in combination with cytokines.

Particularly, the biogel includes TGF-β as the biologically active material. The biogel may be preferably used for treating an injury in a neural tissue or a nervous system (e.g., central or peripheral nervous system) by increasing survival or regenerations of neurons. For example, the biogel including TGF-β may be suitably used in treating the subject or patient having degenerated intervertebral discs (IVD), and the subject or the patient may have been treated or be treated with neurosurgery.

A concentration of the growth factor in the hydrogel composition may range from about 0.1 to about 100 μg/ml, from about 1 to about 100 μg/ml, from about 1 to about 50 μg/ml, from about 1 to about 20 μg/ml, or from about 1 to about 10 μg/ml. In certain embodiments, the concentration of the growth factor in the hydrogel composition is about 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, or 20 μg/ml. In certain embodiments, the concentration of GDF5 in the composition may range from about 0.1 to about 100 μg/ml, from about 1 to about 100 μg/ml, from about 1 to about 50 μg/ml, from about 1 to about 20 μg/ml, or from about 1 to about 10 μg/ml. In certain embodiments, the concentration of GDF in the composition is about 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, or 20 μg/ml. For example, the biogel composition includes the GDF5 at the concentration of about 10 μg/ml.

Suitable biologically active materials (e.g., TGF-β) loaded or incorporated into the biogel compositions may be delivered or administered and may be used in combination with other active or medical agents. Such agents include, but are not limited to, analeptic agents, analgesic agents, anesthetic agents, antiarthritic agents, anti-infective (e.g., antibiotics, antifungal agents, and antiviral agents), anti-inflammatory agents, narcotic antagonists, nicotine, and nutritional agents, (e.g., vitamins, essential amino acids and fatty acids).

In some embodiments, the biologically active material includes a stem cell. For example, the stem cell may promote or induce regeneration of tissue at or around degenerated intervertebral discs (IVD).

In certain embodiments, the biogel may further include one or more of manganese dioxide (MnO2) nanosheets and/or a polymer.

The nanosheets may include a single layer of MnO₂ nanosheet or a multiple layers thereof. Each MnO₂ nanosheet may suitably have a thickness of about 0.5 to 1.5 nm, or around 1 nm. A number of the stacked MnO₂ nanosheets may be controlled to form a desired thickness of the nanosheets. Also, the nanosheets may include MnO₂ nanosheets with or without additional materials disposed therebetween.

The mean size of the nanosheets ranges from about 10 nm to about 1,000 nm, from about 10 nm to about 500 nm, from about 10 nm to about 400 nm, from about 10 nm to about 300 nm, from about 10 nm to about 200 nm, or from about 10 nm to about 100 nm, as measured at the longest dimension of the nanosheets. Preferred mean size of a single nanosheet may range from about 50 to about 100 nm, or about 100 nm. The nanosheets may be stacked in staggered way to optimize inner space between the stacked nanosheets for accommodate therapeutic or biological agents (e.g., biologically active materials) and hydrogel components (e.g., self-assembling peptide).

In certain embodiments, the mean size of a single layer MnO₂ nanosheet may range from about 10 nm to about 1,000 nm, from about 10 nm to about 500 nm, from about 10 nm to about 400 nm, from about 10 nm to about 300 nm, from about 10 nm to about 200 nm, or from about 10 nm to 100 nm. Preferred mean size of the nanosheets may range from about 50 to about 100 nm, or about 100 nm.

The nanosheets as used in the biogel may be biodegradable. The rate of degradation of the MnO2 nanosheets may be controlled by other means, such as for example, controlling the thickness of each MnO₂ layer, the aspect ratio of the total thickness of the nanosheets to surface area of entire nanosheets, presence of additional layers (e.g., adhesion or ionomer layer), presence of other biological material (e.g., spacer proteins, extracellular matrix protein or biodegradable protein), contents of chemical components (e.g., salts, oxidants, reductants, or solvent components), and/or porosity. For example, reducing the thickness of the nanosheets by 5 times can increase the degradation speed by about 3 times; increasing the aspect ratio slows down the degradation speed by over 10 times; and increasing the protein concentrations utilized to assemble nanosheets lead to a significant increase around 7 times.

The rate of the nanosheets may also affect the release of the biological or therapeutic agents (biologically active material) embedded in the nanosheets. For example, the rate at which the therapeutic agent or cells are released from the biodegradable nanoscaffolding is typically substantially equivalent to the rate at which the biodegradable scaffolding material is degraded in vivo. For example, the MnO₂ nanosheets may be controlled to degrade rapidly, with full degradation in one or more days (e.g., 2 to 3 days), or slowly, with around 20% degradation after 2 weeks. In certain embodiments, the degradation rate of the nanosheets may be used for rationally guided drug selection and scaffold design). For example, when the nanosheets are used in the hydrogel composition for treating spinal cord injury (SCI) or intervertebral discs (IVD), slow degradation rate is preferred.

In order to obtain suitably range of physical properties (e.g., stiffness and stability) of the biogel composition in a body (e.g., injury site of a subject), a ratio of the nanosheets to the biogel may be suitably controlled. The weight ratio of the nanosheets to the biogel ranges from 1:10 to 20:1. In certain embodiments, a weight ratio of the nanosheets to the self-assembling peptides is about 1:10, about 1:9, about 1:8, about 1:7, about 1:6, about 1:5, about 1:4, about 1:3, about 1:2, about 1:2.5, about 1:1.5 about 1:1. In certain embodiments, a weight ratio of the nanosheets to the biogel is about 20:1; about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 2.5:1, about 2:1, about 1.5:1, or about 1:1.

The gelatin polymer may be polymerized in the present of the nanosheets. The first gelatin precursor, biologically active material, and the nanosheet may be combined and/or incubated, and then the second gelatin precursor may be added to the mixture to induce biorthogonal reaction.

Alternatively, the second gelatin precursor, biologically active material, and the nanosheet may be combined and/or incubated, and then the first gelatin precursor may be added to the mixture to induce biorthogonal reaction.

In certain embodiments, the second gelatin precursor having a structure of

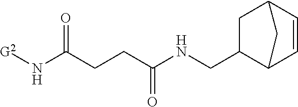

is mixed with the nanosheets and the biologically active material (e.g., drug) and then first gelatin precursor having a structure of

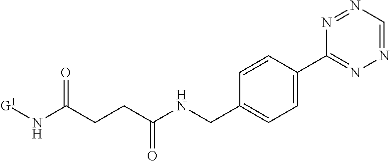

is added to the mixture.

The biogel may further include a matrix. The matrix may include a solvent component (e.g., water, polar aprotic solvent, polar protic solvent, mixtures thereof, or aqueous solution including a pharmaceutically acceptable salt formulation). The matrix may optionally include a polymer that facilitate or promote formation of stable composition. Selection of a solvent component may depend upon the characteristics of the polymer, for example, to provide the secondary forces that stabilize interactions among the polymer, hydrogel, and the nanosheets.

The biogel composition may suitably have a water content greater than about 10% by weight, greater than about 20% by weight, greater than about 30% by weight, greater than about 40% by weight, greater than about 50% by weight, greater than about 60% by weight, greater than about 70% by weight, greater than about 80% by weight, or greater than about 90% by weight. Alternatively, the biogel composition may suitably have a water content of about 10 to 90% by weight, about 10 to 80% by weight, of about 10 to 70% by weight, of about 10 to 60% by weight, of about 10 to 50% by weight, of about 10 to 40% by weight, of about 20 to 80% by weight, about 20 to 60% by weight, about 40 to 80% by weight, or about 40 to 60% by weight.

The biogel compositions may also include additional, optional additive components. Such components may include, for example, permeation enhancers fillers, preservatives, pH regulators, softeners, thickeners, pigments, dyes, refractive particles, stabilizers, toughening agents, detackifiers, and pharmaceutical agents (e.g., antibiotics, angiogenesis promoters, antifungal agents, immunosuppressing agents, antibodies, and the like). These additives, and amounts thereof, are selected in such a way that they do not significantly interfere with the desired chemical and physical properties of the hydrogel composition.

A filler or absorbent filler may be advantageously incorporated to control the degree of hydration when the adhesive is on the skin or other body surface. Such fillers can include microcrystalline cellulose, talc, lactose, kaolin, mannitol, colloidal silica, alumina, zinc oxide, titanium oxide, magnesium silicate, magnesium aluminum silicate, hydrophobic starch, calcium sulfate, calcium stearate, calcium phosphate, calcium phosphate dihydrate, woven and non-woven paper and cotton materials. Other suitable fillers are inert, i.e., substantially non-adsorbent, and include, for example, polyethylenes, polypropylenes, polyurethane polyether amide copolymers, polyesters and polyester copolymers, nylon and rayon.

The biogel compositions can also include one or more preservatives. Preservatives include, by way of example, p-chloro-m-cresol, phenylethyl alcohol, phenoxyethyl alcohol, chlorobutanol, 4-hydroxybenzoic acid methylester, 4-hydroxybenzoic acid propylester, benzalkonium chloride, cetylpyridinium chloride, chlorohexidine diacetate or gluconate, ethanol, and propylene glycol.

The biogel compositions may also include pH regulating compounds. Compounds useful as pH regulators include, but are not limited to, glycerol buffers, citrate buffers, borate buffers, phosphate buffers, or citric acid-phosphate buffers may also be included so as to ensure that the pH of the hydrogel composition is compatible with that of an individual's body surface.

The biogel compositions may also include suitable softening agents. Suitable softeners include citric acid esters, such as triethylcitrate or acetyl triethylcitrate, tartaric acid esters such as dibutyltartrate, glycerol esters such as glycerol diacetate and glycerol triacetate; phthalic acid esters, such as dibutyl phthalate and diethyl phthalate; and/or hydrophilic surfactants, preferably hydrophilic non-ionic surfactants, such as, for example, partial fatty acid esters of sugars, polyethylene glycol fatty acid esters, polyethylene glycol fatty alcohol ethers, and polyethylene glycol sorbitan-fatty acid esters.

The biogel compositions may also include thickening agents. Preferred thickeners herein are naturally occurring compounds or derivatives thereof, and include, by way of example: collagen; galactomannans; starches; starch derivatives and hydrolysates; cellulose derivatives such as methyl cellulose, hydroxypropylcellulose, hydroxyethyl cellulose, and hydroxypropyl methyl cellulose; colloidal silicic acids; and sugars such as lactose, saccharose, fructose and glucose. Synthetic thickeners such as polyvinyl alcohol, vinylpyrrolidone-vinylacetate-copolymers, polyethylene glycols, and polypropylene glycols may also be used.

### METHODS

In an aspect, provided is a method of treating a tissue injury in a subject. The method includes administering the biogel composition as described herein to the subject at the site of the tissue injury.

The subject, e.g., human patient, or non-human subject, may have injury or disorder in a neural tissue, a cartilage, a muscle, or a bone. For example, the subject, e.g., human patient, or non-human subject, has been or is suffering from an injury in a neural tissue, musculoskeletal system (e.g., a muscle, or a bone), or a connective tissue (e.g., cartilage, or ligament).

In certain embodiments, the subject has been or is suffering from an injury in a neural tissue or a nervous system, e.g., central nervous system (CNS) or peripheral nervous system (PNS). For example, the injury is in brain or spinal cord. In certain embodiments, the subject has been or is suffering from degenerated intervertebral discs (IVD). In certain embodiments, the subject has been or is treated with neurosurgery.

The biogel composition is injected or implanted on or around the injured tissue of the subject. The biogel composition may be formulated for injectional administration as described herein.

The biogel composition is gelated or polymerized before the administration or after the administration. The biogel composition is polymerized by crosslinking of the first and second gelatin precursors when it includes them at the predetermined concentration. In certain embodiments, the first gelatin precursor and the second gelatin precursor are sequentially injected or implanted on or around the tissue to form the gelatin polymer to form the gelatin polymer.

The method may further include administering one or more additional therapeutic agents to enhance or synergize the treatment effects.

In an aspect, provided is a kit for treating a tissue in a subject as the methods described herein. The kit includes (i) a first vial including the first gelatin precursor as described herein; (ii) a second vial including the second gelatin precursor as described herein; (iii) the biologically active material as described herein; and an applicator.

The biologically active material may be contained in the first vial and/or second vial. Preferably, the biologically active material may be contained in the second vial. Prior to injection, the first vial and the second vial are combined before injection.

The kit may include one or more of manganese dioxide (MnO₂) nanosheets and/or a polymer contained in the first vial, the second vial, and/or a third vial. Preferably, the nanosheets may be contained in the second or third vial.

The suitable applicator includes an injection syringe that may be attached to a needle, for example, clinically-relevant gauge needles of 25-gauge (diameter: 250 μm), 27-gauge (diameter: 210 μm), or and 30-gauge (diameter: 160 μm). For example, the biogel may be injected via a needle in a syringe subcutaneously (subdermally) to a subject. In certain embodiments, the applicator may include a one- or two-part injection syringe. The applicator may include a two-part injection syringe such that the first gelatin precursor can be added to the second gelatin precursor just prior or at the time of use.

The kit may also include a vial for a dispersion solution (e.g., water, saline solution or suitable dispersion or reconstitution fluid) that may be optionally combined with the biogel composition prior to or at the time of physician's use.

Also provided is a biocompatible tissue implant that includes the biogel as described herein. Further provided herein is a tissue regenerating product including the biogel as described herein.

### EXAMPLES

### Example 1: Preparation of Biogel

### tert-butyl (4-cyanobenzyl)carbamate (tBoc-4CBA) Synthesis

4-(aminomethyl)benzonitrile hydrochloride (1.05 g) was dissolved in 5 mL of water (Solution A). Sodium hydroxide (0.75 g) was dissolved in 5 mL of water. Di-tert-butyl dicarbonate (1.5 g) was added to the solution to form a suspension (Suspension B). Solution A was quickly poured into Suspension B, capped with a rubber septum, and allowed to stir overnight.

The solids were collected and washed with water (2× reaction volume) before placement in a desiccator. A vacuum was drawn to remove residual water overnight to yield the product tert-butyl (4-cyanobenzyl)carbamate (tBoc-4CBA).

### tert-butyl (4-(1,2,4,5-tetrazin-3-yl)benzyl)carbamate (tBoc-Tetrazine) Synthesis

The threads of a 25 mL Duran® laboratory bottle (Schott) were wrapped with PTFE tape to ensure retention of volatile reagents during synthesis. tBoc-4CBA (392 mg), formamidine acetate (1.756 g), and zinc(II) trifluoromethanesulfonate (308 mg) were suspended in dioxane (2 mL) and allowed to stir while hydrazine monohydrate (4.096 mL) was added dropwise. The dark orange/brown solution was transferred to a 60° C. oil bath and allowed to stir for 16 hours.

Pressure was carefully released from the laboratory bottle, and its contents were diluted with ethyl acetate (40 mL). Hydrochloric acid (1 M, 40 mL) was used to extract unreacted reagents with the aid of a separatory funnel, and the aqueous phase was then extracted thrice with ethyl acetate (40 mL). The organic phases were combined with the diluted reaction solution and dried with anhydrous sodium sulfate. A rotary evaporator was used to remove the solvent followed by overnight vacuum drying on a vacuum gas manifold to result in an amorphous orange residue.

Dichloromethane (10 mL) was used to dissolve the orange residue in a glass round bottom flask, and sodium nitrite (2.337 g) was added under stirring to form a suspension. A 50% v/v solution of acetic acid in dichloromethane (20 mL) was slowly added dropwise to the suspension in a well-ventilated chemical safety cabinet with the aid of an addition funnel. During this time, the solution turned bright pink and evolved bubbles.

A cold air purge was used to remove hazardous gases from the flask before diluting with dichloromethane (40 mL). The reaction solution was extracted twice with saturated sodium bicarbonate in water (50 mL per extraction) followed by saturated sodium chloride in water (50 mL per extraction). The organic phase was dried with anhydrous sodium sulfate, placed on a rotary evaporator, and vacuum dried overnight on a vacuum gas manifold to result in an amorphous red solid. The solid was further purified via silica column chromatography using a 5% ethyl acetate in dichloromethane eluent to yield tert-butyl (4-(1,2,4,5-tetrazin-3-yl)benzyl)carbamate (tBoc-Tetrazine) as a red solid.

### 4-(1,2,4,5-tetrazin-3-yl)phenyl)methanamine hydrochloride (Tetrazine Methylamine) Synthesis

tBoc-Tetrazine (75 mg) was dissolved in dichloromethane (4 mL) and allowed to stir while hydrogen chloride in dioxane (4 M, 2 mL) was added dropwise. The reaction solution was capped and allowed to stir for 2 hours. During this time, the solution turned opaque pink.

A rotary evaporator was used to remove the solvent followed by overnight vacuum drying on a vacuum gas manifold to yield 4-(1,2,4,5-tetrazin-3-yl)phenyl)methanamine hydrochloride (Tetrazine Methylamine) as a pink powder.

### Succinylated Gelatin (SA-Gel) Synthesis

Gelatin, type B (1 g) was dissolved in water (24 mL) heated to 37° C. A solution of sodium hydroxide (6 mg) in water (1.5 mL) was added to the gelatin solution and allowed to stir. Succinic anhydride (0.3 g) was added and allowed to react overnight at 37° C.

The reaction solution was neutralized with NaOH in water (1 M) before loading into dialysis bags (nominal MWCO 3.5 kDa). Distilled water was used as the dialysate and changed twice every day for two days. Water was removed via vacuum concentration to yield succinylated gelatin (SA-Gel) as a fibrous white solid.

### Gelatin-Tetrazine and Gelatin-Norbornene (GelTz and GelNb) Synthesis

SA-Gel (240 mg) was dissolved in 2-(N-morpholino)ethanesulfonic acid buffer, pH 6.5 (0.1 M, 24 mL, MES). 1-(3-Dimethylaminopropyl)-3-ethylcarbodiimide hydrochloride (129.6 mg, EDC), N-hydroxysuccinimide (93.6 mg, NHS), and Tetrazine Methylamine (54 mg) were dissolved in separate containers with MES buffer, pH 6.5 (0.1 M, 2.4 mL) immediately before use. The EDC and NHS solutions were added to the SA-Gel solution while stirring and allowed to react for 15 minutes. Tetrazine Methylamine solution was added thereafter, and the solution was allowed to stir for 2 days.

The reaction solution was diluted with water (25 mL) and centrifuged at 6,000 g for 30 minutes to remove aggregates. Desalting and further purification was accomplished using protein concentrators (nominal MWCO 10 kDa) and water (100 mL) according to manufacturer recommended protocol. Care was taken to not allow the protein to exceed 10 mg mL⁻¹ as this increased the risk of protein concentrator fouling. The purified product was removed from the protein concentrators and placed on a vacuum concentrator to yield gelatin-tetrazine (GelTz) as a fibrous pink solid.

Gelatin-norbornene (GelNb) was synthesized and purified with the same protocol except 5-norbornene-2-methylamine (29.72 μL) was added instead of Tetrazine Methylamine to yield a fibrous white solid.

### Fluorescently Labeled Insulin (FITC-Insulin) Synthesis

Insulin (10 mg) was dissolved in sodium carbonate solution (0.1 M, 5 mL, Alfa Aesar), and fluorescein isothiocyanate (1 mg, FITC) was dissolved in anhydrous dimethyl sulfoxide (1 ml). FITC solution (10 drops of 25 μL) was slowly added to the insulin solution under stirring. The reaction vessel was covered from light and allowed to react overnight.

The reaction solution was purified using a protein concentrator (nominal MWCO 3 kDa) and water (25 mL) according to manufacturer recommended protocol. The purified product (FITC-Insulin) was removed from the protein concentrator, tested for concentration using a spectrophotometer (Nanodrop Lite) and A₂₈₀ values derived from protein solutions with known concentrations, and stored at 4° C.

### Fluorescently Labeled Gelatin-Norbornene (FITC-GelNb) Synthesis

GelNb (40 mg) was dissolved in MES buffer, pH 6.5 (0.1 M, 8 mL). EDC (20 mg), NHS (14.4 mg), and ethylenediamine dihydrochloride (20 mg, EDA) were dissolved in separate containers with MES buffer, pH 6.5 (0.1 M, 0.8 mL). The EDC and NHS solutions were added to the GelNb solution while stirring and allowed to react for 15 minutes. EDA solution was added thereafter, and the solution was allowed to stir overnight.

The EDA-GelNb reaction solution was diluted with water (8 mL) and centrifuged at 6,000 g for 30 minutes to remove aggregates. Further purification and buffer exchange was accomplished using protein concentrators (nominal MWCO 10 kDa) and sodium carbonate buffer, pH 9 (0.1 M, 50 mL) to a final volume of 10 mL.

Fluorescein isothiocyanate (FITC) was dissolved in anhydrous DMSO (1 mg mL⁻¹) and used within an hour of mixing. FITC solution (20 aliquots of 20 μL, total 400 μL) was slowly added to the EDA-GelNb solution and allowed to stir overnight.

The reaction solution was diluted with water (5 mL) and centrifuged at 6,000 g for 30 minutes to remove aggregates. Further purification was accomplished using protein concentrators (nominal MWCO 10 kDa) and water (50 mL). The purified product was removed from the protein concentrators and placed on a vacuum concentrator to yield FITC-labeled gelatin-norbornene (FITC-GelNb) as a fibrous red-orange solid.

### Colorimetric Reaction Monitoring

Gelatin, SA-gel, GelTz, and GelNb were dissolved in DPBS (5% w/v) and transferred into a spectrophotometric cuvette. Absorbance measurements were taken with a Cary 60 UV-Vis spectrophotometer (Agilent Technologies).

For colorimetric biogel crosslinking monitoring, GelTz and GelNb were dissolved in DPBS (5% w/v) and quickly mixed before transfer into a spectrophotometric cuvette. Absorbance measurements were taken every minute for 100 minutes using a Cary 60 UV-vis spectrophotometer.

Degree of crosslinking was determined via built-in functions in OriginLab. Absorbance spectra were subjected to Asymmetric Least Squares Smoothing to determine the spectra baseline for each time point. The absorbance peak of tetrazine (k=518 nm) was subtracted from the spectra baseline and normalized to the absorbance peak of tetrazine at T=0 min (i.e., 100% tetrazine unreacted).

### References in Example 1

[1] J. Chin, K. Lang, Methods of incorporating an amino acid comprising a bcn group into a polypeptide using an orthogonal codon encoding it and an orthorgonal pylrs synthase, in: W.I.P. Organization (Ed.) Medical Research Council, 2013.
[2] Y. Wang, J. Ren, Y. Liu, R. Liu, L. Wang, Q. Yuan, J. He, Y. Nie, J. Xu, J. Yu, Preparation and evaluation of folic acid modified succinylated gelatin micelles for targeted delivery of doxorubicin, Journal of Drug Delivery Science Technology 46 (2018) 400-407, https://doi.org/10.1016/j.jddst.2018.06.002.
[3] S. T. Koshy, R. M. Desai, P. Joly, J. Li, R. K. Bagrodia, S. A. Lewin, N. S. Joshi, D. J. Mooney, Click—crosslinked injectable gelatin hydrogels, Adv. Healthcare Mater. 5 (2016) 541-547, https://doi.org/10.1002/adhm.201500757.

### Example 2: Injectable Bioorthogonal Hydrogel (Biogel) Accelerates Tissue Regeneration in Degenerated Intervertebral Discs

Provided herein is a tetrazine-norbornene bioorthogonal ligation combined with gelatin to create an injectable bioorthogonal hydrogel (“biogel”). The liquid hydrogel precursors remain free-flowing across a wide range of temperatures and crosslink into a robust hydrogel after 5-10 minutes, allowing a human operator to easily inject the therapeutic constructs into degenerated IVD. Moreover, the biogel encapsulation of TGF-β potentiated histological repair (e.g., tissue architecture and matrix synthesis) and functional recovery (e.g., high water retention by promoting the matrix synthesis and reduced pain) in an in vivo rat IVD degeneration/nucleotomy model. This biogel procedure readily integrates into existing nucleotomy procedures, indicating that clinical adoption should proceed with minimal difficulty. Since bioorthogonal crosslinking is essentially non-reactive towards biomolecules, this developed material platform can be extended to other payloads and degenerative injuries.

One of the most prevalent causes of back pain is intervertebral disc (IVD) degeneration. IVD degeneration is a progressive and irreversible process causing potential disability as a result of back pain [1, 2]. While growth factor injections have been investigated to encourage matrix synthesis in degenerated IVDs, these injections have not been clinically adopted due to poor efficacy [3]. More specifically, the short half-life of many growth factors [e.g., transforming growth factor beta (TGF-β, implicated in IVD extracellular matrix synthesis) has an in vivo half-life of 2-3 minutes] presents significant limitations to in vivo efficacy [3-5]. Fortunately, growth factors can be loaded into bioscaffolds, resulting in increased bioactivity by modulating release and safeguarding labile payloads from premature degradation [6]. In turn, bioscaffolds provide mechanical stability to the damaged area and act as a template for cells to reconstruct into healthy tissue, allowing the degenerated tissue to be efficiently repaired. [6-10].

To effectively treat IVD degeneration, scaffolds should recapitulate the physicochemical properties of healthy IVD in a clinically translatable manner [11]. These scaffolds serve two key functions: i) regulating stress loading and reducing pain, and ii) delivering biophysical cues to promote pro-regenerative behavior [1, 11]. Several FDA-approved TVD replacements are available, but they primarily comprise metal/synthetic polymer composites that neither recapitulate all biological functions nor mimic the viscoelastic mechanical properties of native IVD tissue [12-14]. While novel biomaterials are being explored for IVD regeneration, many mechanically robust synthetic polymers (e.g., polyethylene glycol, poly lactic-co-glycol acid) display poor biodegradability and/or poor cell adherence [2]. In contrast, various biopolymers (e.g., alginate, chitosan, gellan gum, decellularized IVD) rely on mechanically weak crosslinking modalities [15-17]. Among readily available biomaterials, gelatin-based hydrogels are well-suited for IVD treatment as they retain the unique combination of biocompatibility, biodegradability, cell adhesiveness, cell permissiveness, and viscoelastic properties of its parent structure collagen (a major component of healthy IVD tissue) [10, 18-20]. While unmodified gelatin is unstable at physiological temperatures (i.e., melting at 37° C.), chemical modification with crosslinking groups can impart mechanical stability in vivo while retaining gelatin's beneficial biomaterial characteristics for IVD regeneration [10, 18-20].

Among the many crosslinking modalities reported in the literature, bioorthogonal reactions are of special interest for biomedical applications [10, 18, 19, 21]. These reactions form covalent bonds without damaging or interfering with endogenous tissues [10, 21]. Tetrazine-norbornene ligation represents an ideal bioorthogonal system since these chemical moieties are absent in biological systems, exhibit high selectivity between the two functional groups, crosslink without external triggers or toxic byproducts, and remain stable in aqueous environments [10, 21]. Moreover, tetrazine-norbornene ligation can be adapted to form in situ crosslinking hydrogels, which entail liquid precursors forming solid hydrogels within the body [10, 19, 22]. The clinical benefit of using bioorthogonal, injectable hydrogels for IVD regeneration includes minimizing damage to degenerated IVD tissue, lower costs, shorter post-operative recovery periods, and reduced surgical complications [10, 22, 23].

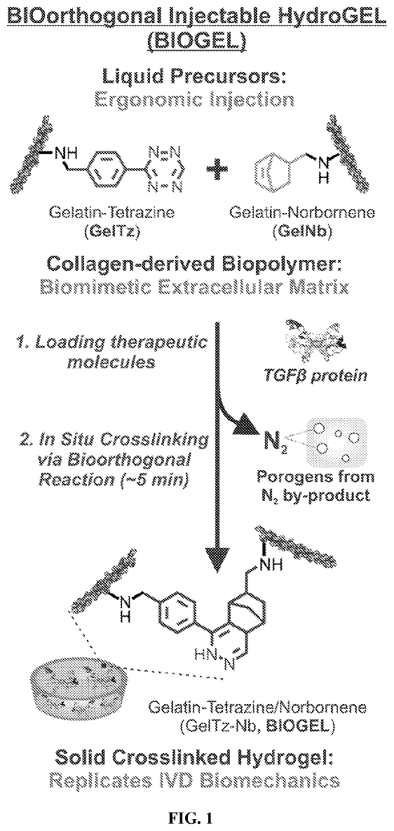

To address the above issues, bioorthogonal chemistry was applied to synthesize an injectable bioorthogonal hydrogel by grafting gelatin with tetrazine and norbornene groups (GelTz-Nb, biogel) (FIG. 1). The resulting biogel system is ergonomic (e.g., it has a low viscosity, which allows for facile injection; it crosslinks within ten minutes of mixing; and it changes color to indicate reaction completion) for ease-of-use in clinical settings, and it mimics the native IVD's biomechanical properties (e.g., viscoelasticity). This recapitulation of IVD biomechanical strength enables the biogel scaffold to withstand mechanical stresses and remain intact until endogenous cells can infiltrate and remodel the hydrogel for IVD regeneration. Given the unique biological, chemical, and mechanical properties of biogel, this injectable hydrogel system was adapted to address the need for effective IVD degeneration treatments. The novel biogel-based therapy described herein included a pro-regenerative cytokine (TGF-β) and a scaffold that replicated IVD biomechanical properties to repair damaged IVD tissue [2, 3, 11, 24]. The injectable hydrogel can protect the labile payload from premature degradation, impart a sustained release profile, and undergo in situ gelation to mechanically reinforce the degenerated IVD. Meanwhile, TGF-β functions as a cell signaling cue to facilitate regeneration and remodel the scaffold [3, 24]. This injectable hydrogel system was then adapted for nucleotomy procedures (removal of nucleus pulposus material through an annulus fibrosus incision) to demonstrate the ease of clinical translation [24, 25]. Collectively, the TGF-loaded biogel treatment was found to improve histological recovery (e.g., tissue architecture and proteoglycan synthesis), which translated into increased functional recovery (e.g., reduced pain) in animal IVD degeneration models.

### MATERIALS AND METHODS

### Materials

Rompun® was purchased from Bayer. RapidCal™ Immuno was purchased from BBC Biochemical. Matrigel® were purchased from Corning. Sprague-Dawley rats were purchased from Orient Bio Inc. Berkovich silicon tips were purchased from Park Systems. Recombinant human FGF-basic (bFGF) was purchased from PeproTech. Complete Nucleus Pulposus Cell Media (CNPCM), human annulus fibrosus cells (hAFCs), and human nucleus pulposus cells (hNPCs) were purchased from ScienCell. B27™ Supplement, human transforming growth factor beta 3 (TGF-β3), lipopolysaccharide (LPS), N2™ Supplement, Neurobasal™ media, and PrestoBlue™ Cell Viability Reagent, was purchased from Thermo Fisher. Zoletil® was purchased from Virbac Laboratories.

Human induced pluripotent stem cell-derived neuronal progenitor cells (hiPSC-NPC) were a generous gift from Dr. Muoti (UC San Diego).

### Precursor and Hydrogel Cytotoxicity

For precursor cytotoxicity with hAFCs and hNPCs, wells of a 96-well plate were coated with PLL as specified by ScienCell for hAFC and hNPC culture. Each well was seeded with hAFCs or hNPCs suspended in either GelTz in DPBS (5% w/v), GelNb in DPBS (5% w/v), or CNPCM (control) (400,000 cell mL⁻¹, 25 μL). After 15 minutes, CNPCM (200 μL) was added to each well.

For hydrogel cytotoxicity with hAFCs and hNPCs, wells of a 96-well plate were coated with either Matrigel® or biogel (25 μL). Matrigel (4° C.) and biogel in DPBS (5% w/v, used within 5 minutes of mixing) were pipetted into their respective wells and allowed to form hydrogels in a humidified, 37° C. incubator for at least 15 minutes before cell seeding. PLL-coated wells (prepared as specified by ScienCell for hAFC and hNPC culture) were used as a control. Each well was seeded with hAFCs or hNPCs (50,000 cell mL⁻¹, 200 μL) suspended in CNPCM.

For hydrogel cytotoxicity with hiPSC-NPC, Matrigel® and biogel-coated wells were prepared as above. Diluted Matrigel®-coated wells (1:200 in DMEM/F-12) were used as a control. Each well was seeded with hiPSC-NPC (5,000 cell mL⁻¹, 200 μL) suspended in proliferation media (0.5% B27, 0.5% N2, 20 ng ml⁻¹ bFGF in 1:1 Neurobasal: DMEM/F-12).

For precursor cytotoxicity with hiPSC-NPC, wells of a 96-well plate were coated with diluted Matrigel®. Each well was seeded with hiPSC-NPC suspended in either GelTz in DPBS (5% w/v), GelNb in DPBS (5% w/v), or proliferation media (control) (5,000 cell mL⁻¹, 25 μL). After 15 minutes, proliferation media (200 μL) was added to each well.

Cytotoxicity was assayed using PrestoBlue™ Cell Viability Reagent after 24-hour culture with hydrogels or precursors. In brief, cells were washed with DPBS to remove excess uncrosslinked GelTz and GelNb before PrestoBlue™ in CNPCM or hiPSC-NPC proliferation media (10% v/v, 200 μL per well) was added. Wells without cells received PrestoBlue™ in CNPCM or hiPSC-NPC proliferation media for background correction. Plates were incubated in a humidified, 37° C. incubator and read using an Infinite M Plex plate reader (Tecan) according to manufacturer specification.

### Field Emission-Scanning Electron Microscopy (FE-SEM) of Crosslinked Hydrogels

Biogel samples were formed using a standardized hydrogel mold. In short, Parafilm was placed over an empty pipette tip rack (5-200 μL, Fisher Scientific), and a multichannel pipette was used to create uniform divots. Equal volumes of hydrogel precursor solutions (25 μL each, 50 μL total) were mixed, pipetted into the molded divots to crosslink for 15 minutes. Crosslinked hydrogels were placed in a lyophilizer (Labconco) overnight to remove water.

Dehydrated samples were attached on SEM specimen mounts with carbon tape and coated with gold (20 nm thickness) using a sputter coater (Quorum Technologies). Electron microscopy was conducted using a secondary electron detector to form images with an FE-SEM (Carl Zeiss AG).

Cross-section samples were generated by cutting lyophilized biogel samples with a stainless-steel razor blade and subjected to the same mounting and coating procedure as described above.

### Rheometry

Rheological measurements were conducted with a Kinexus Ultra rotational rheometer (Malvern Instruments). Solutions (5% w/v, 200 μL distilled water) were loaded into a 600 μm gap between two flat, stainless-steel plates (top 20 mm diameter). GelTz-Nb precursors were mixed and pipetted directly on the rheometer to ensure the cross-linked hydrogel conforms to the top and bottom geometry gap.

Gelatin, SA-Gel, GelTz, GelNb, and GelTz-Nb hydrogel crosslinking were measured with an oscillatory time sweep at a constant 37° C., 1 Hz, 1% shear strain for 100 minutes.

Crosslinked biogel samples were subjected to the following tests: (1) an oscillatory frequency sweep at a constant 37° C., 1% shear strain for 0.01-10 Hz and (2) an oscillatory amplitude sweep at a constant 37° C., 1 Hz for 0.01-10% shear strain.

Gelatin, SA-Gel, GelTz and GelNb were subjected to the following tests: (1) a viscometry temperature sweep at a constant 1 and 100 s⁻¹ for 37-4° C. and (2) an oscillatory temperature sweep at a constant 1 Hz, 1% shear strain for 37-4° C.

### Atomic Force Microscopy (AFM) of Crosslinked Hydrogels

A hydrophobic barrier pen was used to draw a sample retention square on a glass coverslip. Crosslinked hydrogels were placed in the middle of the square to prevent excessive hydrogel movement during microscopy.

AFM (Park Systems) was performed in contact mode with a Berkovich silicon tip at a scan rate of 0.5 Hz over a 5×5 μm area. The hydrogel and probe were kept immersed in distilled water for the duration of probing. Young's modulus was calculated using XEI (Park Systems) software to apply the Oliver-Pharr Model across 10 randomly selected areas. This measurement process was repeated across 3 different regions to yield the reported hydrogel Young's modulus and standard deviation.

FITC-Insulin Release from Biogel

A blocking buffer (1% w/v) was prepared with bovine serum albumin in DPBS. A 24-well plate was pre-treated with blocking buffer (2 mL per well) and placed on a rotary shaker (75 rpm) overnight at 37° C. The blocking buffer was replaced with DPBS (1.5 mL) immediately before use.

Gelatin-Tetrazine (GelTz) and Gelatin-Norbornene (GelNb) were dissolved in either DPBS or fluorescein isothiocyanate-labeled insulin (FITC-Insulin) in DPBS solution (0.25 mg mL⁻¹) with the aid of a sonicator bath to yield hydrogel precursor solutions (5% w/v). A hydrogel mold was made by spreading Parafilm over an empty pipette tip rack (5-200 μL, Fisher Scientific) and using a multichannel pipet to create uniform divots. Equal volumes of hydrogel precursor solutions (25 μL each, 50 μL total) were mixed, pipetted into the molded divots to crosslink for 15 minutes, and transferred to the pre-treated 24-well plate. The well plate was covered with aluminum foil and sealed with Parafilm before placement on a rotary shaker (75 rpm) set in a 37° C. incubator. An aliquot of FITC-Insulin solution was placed in a glass dram vial, covered with aluminum foil, and placed alongside the well plate for the duration of the experiment.

Fluorescence measurements were taken at 1 hour, 4 hr, 1 day, 2 d, 3 d, 1 week, and 2 wk using a plate reader (Tecan Infinite M200 Pro) at λₑₓ=488 nm, λₑₘ=525 nm. An aliquot (100 μL) was taken from each well without replacement and placed in a half area, black well plate for fluorescence readings. A standard curve was created by serial dilution of the co-incubated FITC-Insulin solution at each time point to calculate % FITC-Insulin release.

### Collagenase Biodegradation Assay

A blocking buffer (1% w/v) was prepared with bovine serum albumin in DPBS. A 24-well plate was pre-treated with blocking buffer (2 mL per well) and placed on a rotary shaker (75 rpm) overnight at 37° C. The blocking buffer was replaced with DPBS (1 mL) immediately before use.

GelTz was dissolved in DPBS (5% w/v) in a microcentrifuge tube, while GelNb and FITC-GelNb (9:1 w/w ratio) were dissolved in DPBS (total 5% w/v) in a separate tube to yield the hydrogel precursor solutions. A hydrogel mold was made by spreading Parafilm over an empty pipette tip rack (5-200 μL, Fisher Scientific) and using a multichannel pipet to create uniform divots. Equal volumes of hydrogel precursor solutions (25 μL each, 50 μL total) were mixed, pipetted into the molded divots to crosslink for 15 minutes, and transferred to the pre-treated 24-well plate. The well plate was covered with aluminum foil and sealed with Parafilm before placement on a rotary shaker (75 rpm) set in a 37° C. incubator. An aliquot of FITC-GelNb solution was placed in a glass dram vial, covered with aluminum foil, and placed alongside the well plate for the duration of the experiment.

DPBS was replaced every day for two days to leach any unreacted/uncrosslinked FITC-GelNb. Leaching solutions were assayed with a plate reader (Tecan Infinite M200 Pro) at λₑₓ=488 nm, μₑₘ=525 nm to determine the concentration of unreacted FITC-GelNb.

Meanwhile, collagenase type III was reconstituted in DPBS (10 mg mL⁻¹). Aliquots were made and subjected to at most two freeze-thaw cycles prior to use. After the second DPBS leaching procedure, the biogel samples were resuspended in collagenase solution (100, 10, or 1 μg mL⁻¹, 1.5 mL). Fluorescence measurements were taken at 1 hour, 4 hr, 1 day, and 2 d. An aliquot (100 μL) was taken from each well without replacement and placed in a half area, black well plate for fluorescence readings. A standard curve was created by serial dilution of the co-incubated FITC-GelNb solution at each time point to calculate % FITC-GelNb release/biogel degradation after correcting for the unreacted FITC-GelNb mass.

### In Vitro TGF-β Release for Anti-Inflammatory Response

PLL-coated wells of a 24-well plate were seeded with hAFCs and hNPCs resuspended in CNPCM (200,000 cell mL⁻¹, 0.5 mL) and allowed to stabilize overnight. Likewise, Matrigel®-coated wells were seeded with hiPSC-NPC in proliferation media in a similar manner.

TGF-β3 was diluted in DPBS (0.15 mg mL⁻¹) before addition to GelTz in DPBS (5% w/v) for a final concentration of 75 μg mL⁻¹ TGF-β3 (TGF-β3+GelTz). TGF-β3 was excluded from the control unsupplemented biogel; only DPBS was used to dilute the stock GelTz (5% w/v) solution.

(TGF-β3+) GelTz (16.56 μL) and GelNb (5% w/v, 12.5 μL) solutions were mixed and allowed to form hydrogels in the Parafilm divots at least 15 minutes prior to being introduced to cells. hAFC and hNPC media was replaced with LPS-containing CNPCM (10 μg mL⁻¹, 0.6 mL) to simulate intervertebral disc degeneration. Likewise, hiPSC-NPC media was replaced with LPS-containing proliferation media at the same concentration. A cell culture insert (0.4 μm pore size, polycarbonate, 6.5 mm) was added to each well and filled with LPS-containing media (10 μg mL⁻¹, 0.1 mL). In non-LPS-containing controls, LPS was omitted from the media.

In control and TGF-β3-treated wells, DPBS or TGF-β3 (0.25 mg mL⁻¹ TGF-β3, 2.5 μL) was added to the cell culture inserts. In (TGF-β3+) biogel-treated wells, appropriate hydrogels were transferred into the cell culture inserts. After 24 hours, cell contents were harvested for RT-qPCR with primers below.

TABLE 1
RT-qPCR primer sequences

| Gene | Forward | Reverse |
|---|---|---|
| Name | Primer (5′-3′) | Primer (5′-3′) |
| GAPDH | CAT GTT CCA ATA | GAT GGG ATT TCC |
| | TGA TTC CAC C | ATT GAT GAC |
| | (SEQ ID NO: 1) | (SEQ ID NO: 2) |
| CXCL1 | TCA CAG TGT GTG | AGC CCC TTT GTT |
| | GTC AAC AT | CTA AGC CA |
| | (SEQ ID NO: 3) | (SEQ ID NO: 4) |
| IL-6 | AAA CAA CCT GAA | GCA AGT CTC ATT |
| | CCT TCC AAA GA | GAA TCC A |
| | (SEQ ID NO: 5) | (SEQ ID NO: 6) |
| MMP13 | CCC AAC CCT AAA | AAA CAG CTC CGC |
| | CAT CCA A | ATC AAC C |
| | (SEQ ID NO: 7) | (SEQ ID NO: 8) |
| CGRP | ATG CAG CAC CAT | CCA GCC GAT GAG |
| | TCA GGT CTG | TCA CAC AG |
| | (SEQ ID NO: 9) | (SEQ ID NO: 10) |
| Substance | CGA CAG CGA CCA | TGC ATT GCA CTC |
| P | GAT CAA GGA GG | CTT TCA T |
| | (SEQ ID NO: 11) | (SEQ ID NO: 12) |

### Animal Model for Intervertebral Disc (IVD) Injury and Treatment

All animal study procedures were approved by the Institutional Animal Care and Use Committee (IACUC) of CHA Bundang Medical Center (IACUC200141).

Eight-week-old Sprague-Dawley rats (220-240 g) were kept in a controlled environment (22±1° C., 50±1% relative humidity, and 12/12 hr light/dark cycle) for the duration of the study. Animals were anesthetized by intraperitoneal injection of Zoletil® (50 mg kg⁻¹) and Rompun® (10 mg kg⁻¹) before being placed on heated pads for the duration of surgery. The tail and pelvic area were sterilized with 70% alcohol, followed by povidone iodine. A skin incision (1 cm) was made longitudinally along the tail followed by the insertion of a scalpel blade (#11, 1.5 mm) into coccygeal discs Co4-5 and Co 5-6. The annulus fibrosus was longitudinally cut to expose the NP for nucleotomy via aspiration (22-gauge needle, 5 ml syringe). biogel, TGF-β, and TGF-β loaded biogel (5% w/v in DPBS) were prepared and intradiscally injected into the Co 4-5 nucleotomy site (10 μL, 25-gauge catheter).

The surgical site was sutured and disinfected with povidone-iodine before subcutaneous injection of normal saline (0.9%, 5 mL). For 3 days post-surgery, animals were prophylactically treated with Cefazolin and Ketoprofen.

Twenty-four Sprague Dawley rats were subjected to nucleotomies at coccygeal vertebrae 4-5 and 5-6 (Co4-5 and Co5-6), with Co4-5 receiving TGF-β, biogel, or TGF-β loaded biogel treatments. Co3-4 was designated as the non-injured control. The rats were randomly divided into three groups: (1) TGF-β, (2) biogel, and (3) TGF-β loaded biogel treatment groups (n=8 for each group). Forty two days after treatment, the coccygeal discs were removed for radiologic and histologic analysis.

### Histological Recovery Assessments of IVD Regeneration

Animals were sacrificed six weeks post-surgery to evaluate tissue structure and morphology. IVD and adjacent vertebral bodies were fixed in neutral buffered formalin (10%) for one week, decalcified with RapidCal Immuno for two weeks, paraffin-embedded, sliced using a microtome (Leica) to produce coronal sections (10 μm), dewaxed, and rehydrated.

Tissue architecture was determined by Safranin-O and Mayer's Hematoxylin & Eosin staining. Sections were mounted using mounting media and scanned with a microscope (OLYMPUS).

Built-in ImageJ region of interest functions were used to determine the NP area. Additionally, IVD tissue architecture was graded by blinded pathologists for the following criteria as detailed elsewhere (0 for healthy disc, 2 for severely injured disc): AF morphology (score 0-2), NP cellularity (score 0-2), NP matrix (score 0-2), and the boundary between AF and NP (score 0-2) [24]. The IVD tissue architecture score is presented as an average of the four criteria.

Specific markers were determined via immunostaining with antibodies below.

TABLE 2
Immunostaining reagents and catalog numbers

| Antigen | Conjugated | Catalog # | Dilution |
|---|---|---|---|
| Aggrecan | | ab36861 (Abcam) | 1:1000 |
| Collagen Type 2 | | ab34712 (Abcam) | 1:100 |
| CGRP | | ab47027 (Abcam) | 1:200 |
| Iba1 | | ab 107159 (Abcam) | 1:200 |
| CD86 | | ab213044 (Abcam) | 1:200 |
| CD163 | | ab182422 (Abcam) | 1:200 |
| Rabbit IgG | Horseradish | 05271967001 (Roche) | (Kit) |
| | Peroxidase | | |
| Rabbit IgG | Alexa Fluor ® 488 | A11034 (Invitrogen) | 1:400 |
| Mouse IgG | Alexa Fluor ® 488 | A11029 (Invitrogen) | 1:400 |
| Rabbit IgG | Alexa Fluor ® 568 | A10042 (Invitrogen) | 1:400 |
| Goat IgG | Alexa Fluor ® 647 | A21469 (Invitrogen) | 1:400 |
| | DAPI | D1306 (Invitrogen) | 1:500 |

Sectioned samples were incubated with primary antibodies at 4° C. for overnight, washed with DPBS and Tween 20, incubated with secondary antibodies at room temperature for one hour, washed, and counter-stained with DAPI for 10 minutes. Sections were mounted using mounting media and scanned with a fluorescence microscope (Zeiss).

Built-in ImageJ region of interest functions were used to determine positive staining area and relative cell number percentages.

### Functional Recovery Assessments of IVD Regeneration

Von Frey tests were conducted −2 [pre-injury], 2, 7, 14, 21, 28, 35, and 42 days after surgery to assess mechanical allodynia as pain behavior in rats. Animals were placed into individual six-compartment enclosures with wire mesh floors and lids with air holes for a habituation period (20 minutes) to minimize exploratory activity. Von Frey hairs (starting with a 2-g filament) were applied to the ventral surface of the tail base with sufficient force to buckle the filament. Positive responses were recorded if animals displayed flinching, licking, withdrawing, or tail shaking behaviors within 6 seconds of filament application. Tests were conducted five times for each rat with two blinded observers.

A T2-weighted, 9.4 T MRI spectrometer (Bruker BioSpec) was used to study the changes in disc structure and water content 42 days after surgery. T2-weighted MRI protocol was set as: (1) coronal plane; time to repetition (TR) of 5000 ms, time to echo (TE) of 30 ms, 150 horizontal×150 vertical matrix; field of view of 15 horizontal 15 vertical, and 0.5 mm slices with 0 mm spacing between each slice. (2) Sagittal plane; time to repetition (TR) of 5000 milliseconds (ms), time to echo (TE) of 50 ms, 200 horizontal_600 vertical matrix; field of view of 20 horizontal_60 vertical, and 0.8 mm slices with 0 mm spacing between each slice. The signal intensity and MRI index (the area of NP multiplied by average signal intensity) were evaluated [47]. Built-in ImageJ region of interest functions were used to determine the signal intensity (product of NP area and average signal intensity) while two blinded observers were used to determine the MRI index as detailed elsewhere [43].

### Statistical Analysis

In vitro RT-qPCR data was analyzed with R (statistical program), while in vivo data was analyzed with GraphPad Prism. All data are visualized as mean±standard deviation. Statistical significance was defined as p-value<0.05 when analyzed by one-way ANOVA followed by Tukey's HSD test.

### Computer Graphics

OriginLab was used for data analysis and graphing unless indicated otherwise. TGF-β and gelatin figures were obtained from RCSB PDB (PDB ID: 1TGK and 1BKV) and visualized using Mol* [48-52]. Select graphics were created with BioRender.com.

### Results and Discussion

### Biogel Synthesis and Physicochemical Characterization

Gelatin was selected to form the injectable hydrogel since it retains many biological functions of its parent structure, collagen (a major component of healthy IVD tissue) [20]. Since gelatin melts at physiological temperatures, a crosslinking mechanism is required to impart stability during IVD regeneration [20]. Tetrazine and norbornene were chosen as the bioorthogonal functional groups because of their simple synthesis, favorable reaction kinetics, and essentially non-reactivity toward endogenous biomolecules [10, 21]. Moreover, the crosslinking reaction generates non-toxic byproducts and is easily initiated by simple mixing [10]. The resulting gelatin-tetrazine (GelTz) and gelatin-norbornene (GelNb) is a shelf-stable solid that can be freely reconstituted. Once mixed, GelTz and GelNb solutions form the biogel hydrogel at ambient temperatures without substantial interference from other biomolecules. This bioorthogonal reaction was adapted to form an injectable hydrogel system, where the liquid precursors are injected in vivo and allowed to crosslink in situ.

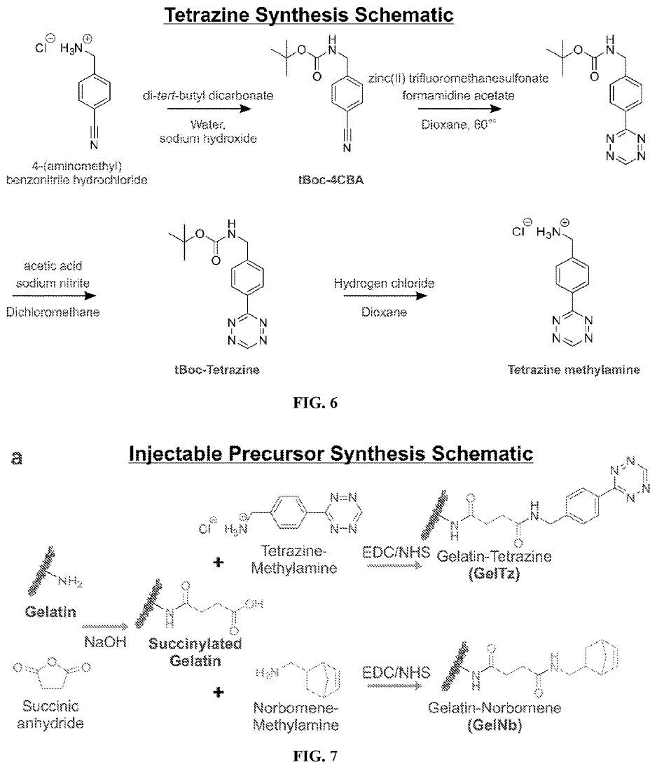

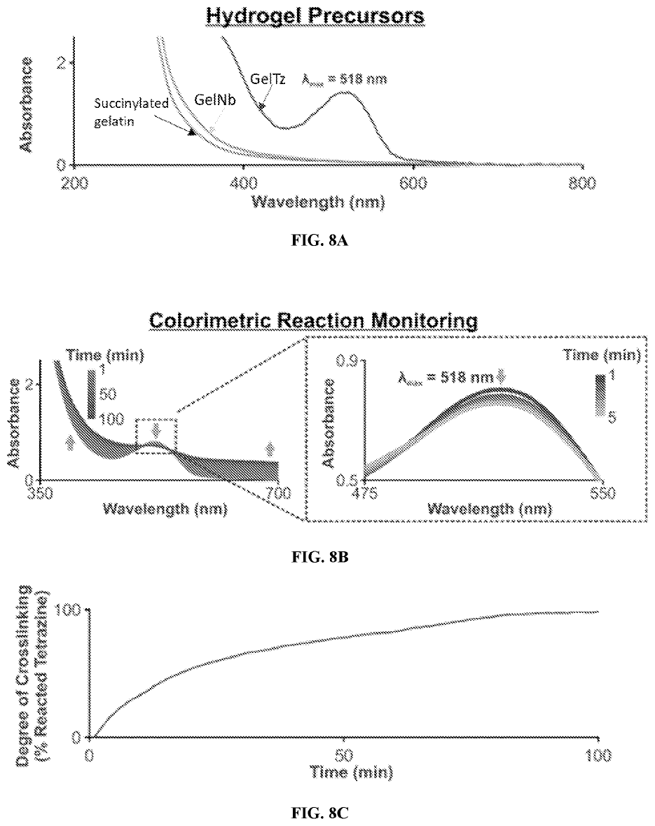

Gelatin natively contains functional groups for bioconjugation. Norbornene-Methylamine (5-norbornene-2-methylamine) is commercially available, while Tetrazine-Methylamine [4-(1,2,4,5-tetrazin-3-yl)phenyl)methanamine hydrochloride] synthesis (FIG. 6) and nuclear magnetic resonance (NMR) are found in Supplementary Information [26]. GelTz and GelNb synthesis schematics are found in FIG. 7. First, gelatin was modified with succinic anhydride to (1) increase available carboxylic acid groups for further modifications, (2) prevent amine-carboxylic acid cross-coupling within gelatin molecules, and (3) reduce shear viscosity of the resulting precursor solution. Carboxylic acid groups on the succinylated gelatin were conjugated with Tetrazine-Methylamine and Norbomene-Methylamine via carbodiimide chemistry to form GelTz and GelNb. NMR confirmed the addition of succinate groups, tetrazine, and norbornene. The conjugated π-π system of Tetrazine-Methylamine gives GelTz a bright red color with an absorption maximum at 518 nm (FIG. 8A). This permits facile visualization of the injected hydrogel as it crosslinks, since the 518 nm peak diminishes as the reaction progresses (FIGS. 8B, 8C). When tactile feedback is limited (e.g., laparoscopic surgeries), this additional means of verifying successful biogel crosslinking is desirable to (1) indicate when mixed GelTz-Nb solution is no longer injectable and (2) confirm gelation once injected into the patient.

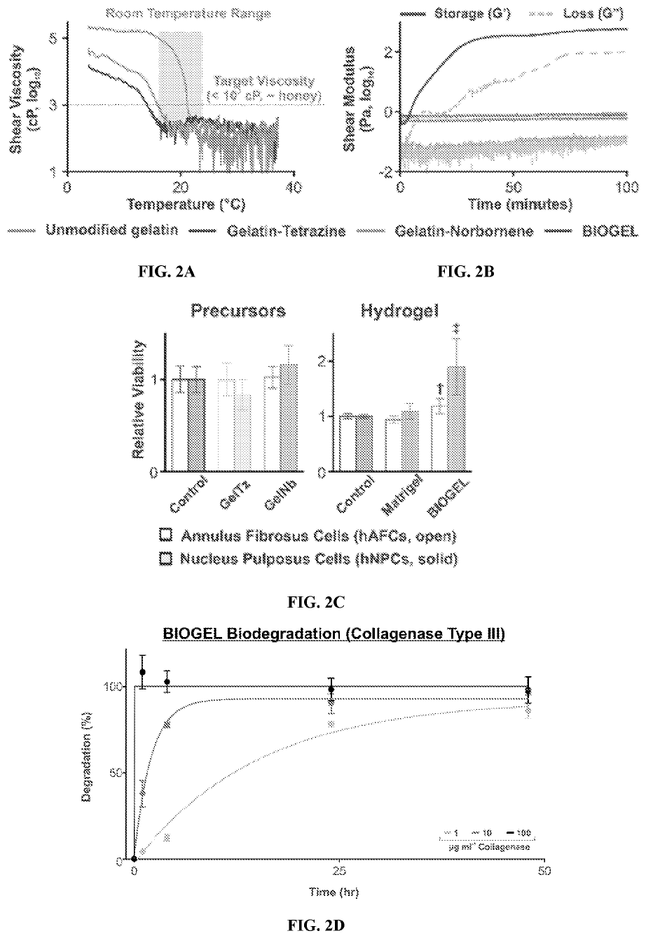

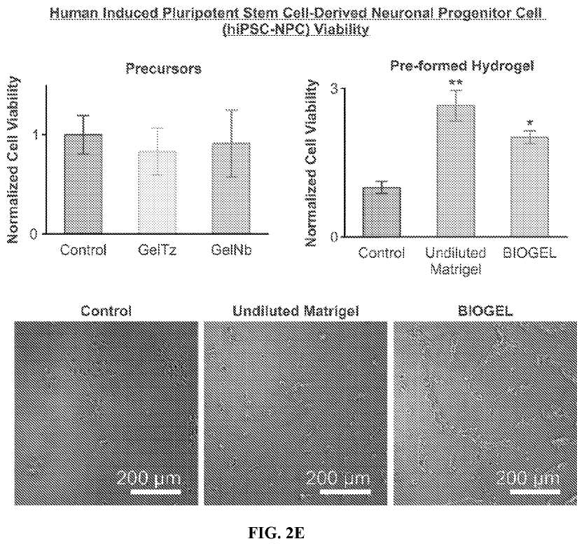

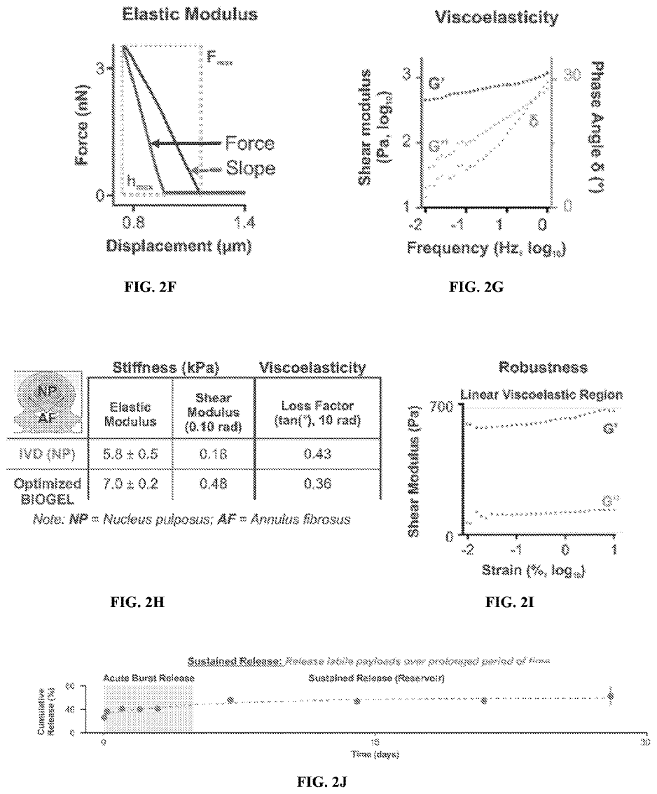

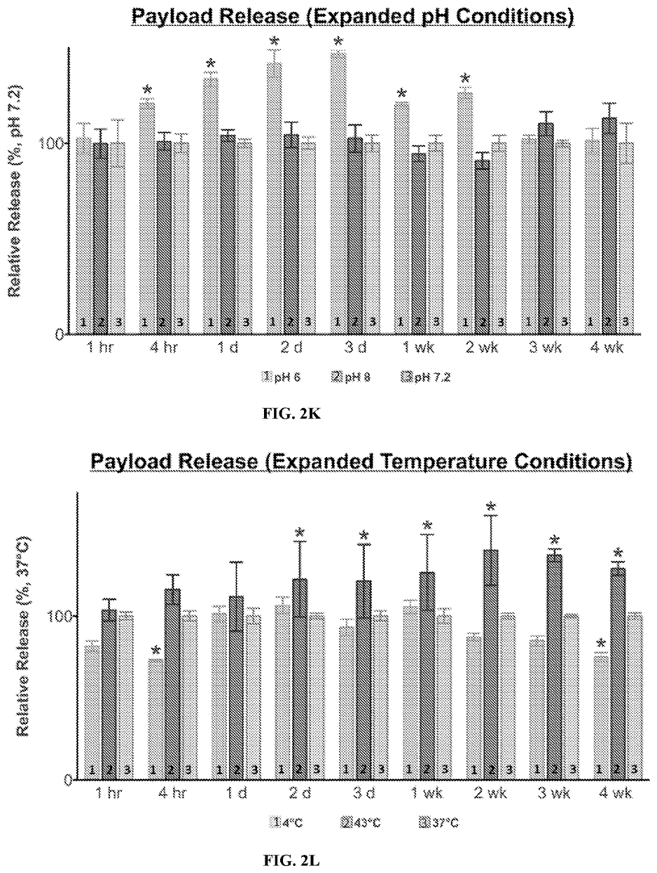

Ease of injection is paramount for an injectable hydrogel system, particularly considering constraints present in a surgical site (e.g., space for additional equipment). The injectable hydrogel should keep a low viscosity so that it can be easily delivered to the patient, the amount of force needed for injection can be minimized, and shear stresses can be reduced [7, 8]. Biopolymers may exhibit significantly increased viscosity when placed in non-optimal conditions, thus hindering injection. Unmodified gelatin solutions became significantly more viscous at room temperature (FIG. 2A), potentially necessitating additional equipment to maintain physiological temperatures prior to injection [20]. GelTz and GelNb solutions exhibited low viscosities across a wider range of temperatures (e.g., honey at 37° C. has a viscosity of approximately 10³ cP). These results were also seen when measuring the shear modulus of the precursor, whereby the GelTz and GelNb solutions exhibited lower moduli than gelatin or succinylated gelatin. With a lower viscosity, less force is required to pass the precursor solutions through a syringe, and any encapsulated payloads (e.g., sensitive proteins or cells) will experience less damaging shear stresses during injection [7]. Thus, ease of injectability is maintained across a wide temperature range for the biogel hydrogel system, and additional equipment is not required to maintain optimal injection temperatures.

Ergonomic design and use are important considerations when translating a new therapy to the clinic. A crosslinking-based injectable hydrogel must react at an appropriate timescale. Excessively fast reactions can be difficult when administering the hydrogel before complete gelation [6]. Unacceptably slow reactions can be subjected to premature clearance by local fluid flow and increase healthcare costs related to prolonged operations [6]. biogel gelation (i.e., sustained increase in shear modulus) began approximately 5 minutes after precursor mixing with a plateau after 95 minutes (FIG. 2B). In vivo experiments were usually concluded (i.e., closing the surgical site) before the gelation plateau was complete. As a control, unmodified gelatin and individual precursor solutions were subjected to the same rheometer time sweep sequence and failed to achieve any substantial gelation. The delay in gelation was deemed appropriate for clinical use as it gave the operator ample time to thoroughly mix the precursor solutions, load into a syringe, and administer to the surgical site.

While tetrazine-norbornene ligation is bioorthogonal and biogel uses a biomimetic extracellular matrix (ECM), it is still prudent to establish the cell adhesiveness, biocompatibility, and biodegradability of both the precursor solutions and biogel. Cytotoxicity assays on human annulus fibrosus cells (hAFCs) and human nucleus pulposus cells (hNPCs) showed no significant cell death when (1) precursor solutions are compared to media or (2) biogel is compared to conventional cell culture on poly-L-lysine (PLL)-coated tissue culture plastic control or Matrigel hydrogels (FIG. 2C). In fact, biogel was found to promote hAFC and hNPC proliferation. When monitoring the morphology of cells on PLL, Matrigel, and biogel, an interesting observation was seen in hNPCs. Matrigel induced hNPCs to form cell clusters, an in vivo indication of IVD degeneration, whereas cells on biogel maintained conventional monolayer culture (not shown) [24]. In addition, crosslinked biogel samples were verified to be biodegradable via collagenase digestion (FIG. 2D). Lastly, both the biogel precursors and hydrogel were also found to be non-toxic to human-induced pluripotent stem cell-derived neuronal progenitor cells (hiPSC-NPC, FIG. 2E), indicating biocompatibility with neuronal tissue in the nearby spinal cord.

FE-SEM revealed that biogel contained pores larger than 100 μm (the upper bound for most mammalian cells), even after dehydration for electron microscopy. These pores were seen both on the surface of the hydrogel and when it was cross-sectioned. Porous hydrogels are well-documented to promote nutrient exchange and encourage host cell infiltration and integration, thus providing resident cells with a favorable microenvironment to remodel the injectable hydrogel [7, 9]. The combination of high biocompatibility, high cell adhesiveness, and high porosity enables host cells to readily infiltrate the hydrogel for eventual remodeling and tissue regeneration.

Since one of the key IVD functions is load transmission throughout the spinal column, the mechanical properties of biogel are important for in vivo function. Similarity to healthy human IVD tissue is significant as it assists with regulating cellular mechanotransduction, host tissue integration, and normal loading bearing (i.e., lack of stress shielding) [27, 28]. Atomic force microscopy (AFM) was employed to obtain a detailed image of biogel when hydrated and to determine the elastic modulus (FIG. 2F), while rheometry was used to determine viscoelastic behavior under shear stresses (FIG. 2G). Biogel exhibited elastic modulus, shear modulus, and loss factors similar to healthy IVD tissue (FIG. 211) [29, 30]. In comparison, current FDA-approved treatments exhibit significantly higher elastic modulus than healthy IVD (e.g., elastic modulus on the order of GPa) [12-14]. Moreover, oscillatory frequency sweeps indicated that biogel viscoelasticity allows for the overall structure to remain unchanged at low frequencies (elastic behavior) while undergoing dynamic reorganization in response to high frequency (viscous behavior) [28]. In practice, this viscoelasticity allows the hydrogel to maintain its shape for low-frequency forces (e.g., standing at rest) and deform at higher frequencies (e.g., rapid spine extension and rotation). Additionally, the hydrogel retained its mechanical properties under a wide range of strains (0.01-10% strain, termed linear viscoelastic region), showing that the hydrogel does not undergo structural breakdown and has high wear resistance (FIG. 2I) [28]. Taken together, the biogel system performs similarly to healthy IVD tissue under key mechanical loading situations expected when implanted in vivo.

### Biogel Enhances In Vitro Efficacy of Growth Factor Therapy

While biogel can provide mechanical support to degenerated IVD, it is preferable to concurrently induce nearby cells to remodel the hydrogel and regenerate damaged tissue. Growth factor therapies are suited to initiate IVD regeneration but suffer from significant issues that limit clinical efficacy [5]. For TGF-β, a well-known cytokine that is implicated in ECM remodeling and anti-inflammation, its short in vivo half-life severely limits its clinical potential as a bolus injection [3-5, 31-33]. Potential benefits may be found by encapsulating TGF-β with the biogel system to reverse IVD degeneration.

The release of encapsulated payloads in biogel was modeled using FITC-labeled insulin (similar size to TGF-β). Since GelTz and GelNb can be dehydrated into a shelf-stable solid, FITC-insulin was integrated into the hydrogel by reconstituting GelTz in a FITC-insulin solution. A biphasic release profile was seen where burst release within the first five days transitions into a more gradual, sustained release (FIG. 2J). This biphasic drug release behavior persisted under various physiological conditions including tissue acidosis, alkalosis, hypothermia, and hyperthermia (FIGS. 2K, 2L). Moreover, a portion of FITC-insulin (approximately 40% of the original loading mass) remained trapped in the hydrogel under normal physiological conditions. The presence of unreleased payload (e.g., TGF-β) represents a significant benefit over conventional growth factor therapies. Whereas injected TGF-β has a short half-life and is rapidly cleared, a significant portion of TGF-β within the hydrogel is sequestered until cells infiltrate and release the trapped payload [3-5, 24]. biogel protects against premature clearance and acts as a reservoir of sequestered TGF-β, while the growth factor induces cells to remodel the hydrogel, abate pro-inflammatory signals in the environment, and initiate pro-regenerative cascades [3, 5, 6].

To verify the biogel system's capacity to enhance growth factor therapies, hAFCs, hNPCs, and differentiated hiPSC-NPC neurons were treated with lipopolysaccharide (LPS) to simulate an inflammatory state comparable to IVD degeneration [34, 35]. In hAFCs and hNPCs, this testing condition promotes the production of IVD degeneration markers such as C-X-C Motif Chemokine Ligand 1 (CXCL1), Interleukin-6 (IL-6), and Matrix Metalloprotease 13 (MMP13) [36]. Similarly, hiPSC-NPC neurons express pain-related genes such as calcitonin gene-related peptide (CGRP) and Substance P [37]. These LPS-activated signaling pathways can be blocked by administering TGF-β to induce anti-inflammatory cascades, leading to reduced IVD degeneration and pain [32, 33]. While free, unreacted gelatin is well-known for activating integrin receptors, its net effect on inflammation is less predictable as it can activate both pro- and anti-inflammatory pathways [38].

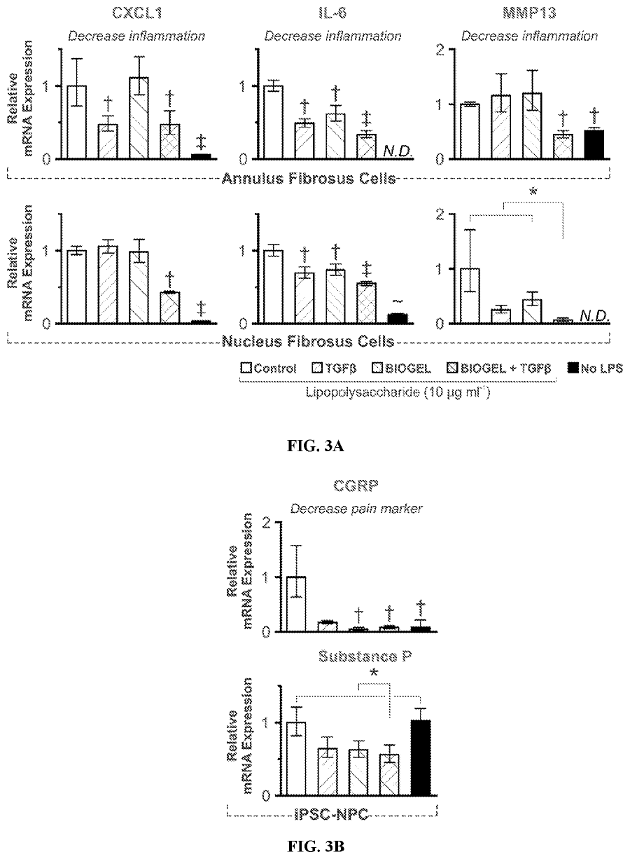

TGF-β solution, bare biogel, and TGF-β loaded biogel (“TGF-β biogel”) were administered to LPS-primed cells via cell culture inserts (0.4 μm pore to allow diffusion from the treatment chamber to cells) to test their ability to mitigate the pro-inflammatory, pain-inducing effects of LPS. After 24 hours, the various inflammatory and pain markers were found to be significantly reduced when cells were treated with TGF-β biogel (FIGS. 3A-3B). Though the reduction usually could not achieve gene expression levels found in the non-inflammatory control, these showed significant potential for TGF-β biogel to reduce IVD degeneration and pain. Moreover, TGF-β biogel was found to reduce inflammatory and pain markers with similar or greater efficiency than either TGF-β or biogel component by itself, suggesting that biogel encapsulation potentiates therapeutic payload effects. While LPS was insufficient at inducing Substance P upregulation here, the ability of TGF-β biogel to downregulate baseline expression is significant enough to warrant interest in IVD treatment.

Several ECM-related genes were also assayed in hAFCs and hNPCs (i.e., Aggrecan, Collagen Type 2) but failed to yield sufficient PCR amplification (data not shown). Dedifferentiation and loss of ECM-related gene expression are well documented for primary IVD cells after extended monolayer culture [39].

### Biogel Enhances In Vivo Histological Recovery

IVD degeneration is a complex pathology that necessitates in vivo experiments to assess novel therapies. Nucleotomy (removal of nucleus pulposus material [NP] through an annulus fibrosus [AF] incision) was selected as the IVD degeneration model and method of biogel injection due to established clinical use in conditions such as lumbar microdiscectomy, which is the most commonly performed spinal surgery procedure [25]. Sprague Dawley rats were subjected to nucleotomies at coccygeal vertebrae 4-5 and 5-6 (Co4-5 and Co5-6), with Co4-5 receiving TGF-β, biogel, or TGF-β loaded biogel treatments. Co3-4 was designated as the non-injured control. Forty-two days after treatment, the rats were sacrificed for tissue harvesting and histological assessment of the IVD. Healthy IVD tissue contains distinct boundaries between the AF and NP, while degenerated IVD lacks a clear boundary [24]. Hematoxylin and Eosin (H&E) staining was used to visualize this boundary as the interface between the pink, eosinophilic AF and purple, hematoxylin-staining NP [24, 40]. Additionally, proteoglycan-rich NP stains red, while the AF stains blue-green under Safranin-O/Fast Green [24, 40]. Together, these staining techniques were used to elucidate the extent of IVD regeneration.

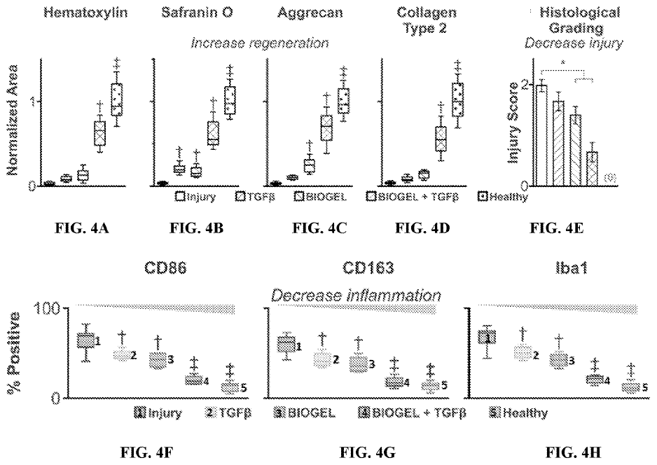

Healthy IVD discs retained distinct AF/NP regions, while injured IVD showed diminished boundaries. While TGF-β and bare biogel, as control conditions, in the Co4-5 IVD showed negligible to modest recovery of AF-NP boundaries, TGF-β loaded biogel displayed substantial histological recovery as quantified by hematoxylin and Safranin O-stained area (FIGS. 4A, 4B) and histological grading (FIG. 4C). The recovery of distinct AF/NP boundaries and proteoglycan content in the NP indicated regeneration [24, 40]. More specifically, the recovered proteoglycan content helped the IVD retain water and resist compressive forces during load bearing [11]. Since biogel contained fewer proteoglycans than the TGF-β loaded biogel, the injected biogel potentiates encapsulated growth factor therapies (e.g., TGF-β) to induce native cells to remodel the hydrogel.

Tissue and organ functions, particularly those under mechanical forces, are intrinsically linked with tissue composition and architecture [1, 11]. Cartilaginous tissues, such as the IVD, need to resist various mechanical forces while remaining pliable enough to allow movement [1, 11]. Collagen type 2 and Aggrecan are two cornerstones of IVD composition and function [1, 11, 40]. Immunohistochemistry staining for Collagen Type 2 and Aggrecan showed ample staining, primarily localized on the NP. TGF-β notably failed to induce significant recovery of either ECM component after injury (results mirrored in literature) [3]. While bare biogel enabled modest recovery of Aggrecan synthesis, treatment with TGF-β loaded biogel promoted the greatest recovery of matrix (Aggrecan and Collagen type 2) synthesis (FIGS. 4C, 4D). Holistic IVD evaluation using a histological grading scheme (details in literature reference) also corroborated the conclusion of enhanced tissue recovery when patients are treated with TGF-β loaded biogel (FIG. 4E) [24, 40]. Taken together with H&E and Safranin-O staining, these results provide ample evidence that TGF-β loaded biogel induced significant recovery of key histological features of the IVD, including key proteoglycans and structural proteins. Moreover, treatments with TGF-β solution or biogel alone failed to produce consistent recovery.

Concurrent with tissue damage, IVD degeneration is associated with inflammation [41, 42]. While numerous cells are involved in inflammation, macrophages (identified by Iba1) remain an integral mediator across many tissues [41, 42]. M1 macrophages (CD86-positive) are associated with a pro-inflammatory state which can lead to further tissue damage, while M2 (CD163-positive) is associated with anti-inflammation and regenerative processes such as tissue remodeling and matrix deposition [41, 42]. In injured IVD, a significant number of macrophages infiltrated the damaged tissue (FIGS. 4F, 4G, 4H). While TGF-β and bare biogel resulted in a moderate decrease in macrophages, TGF-β loaded biogel treatment produced the greatest reduction. Notably, the difference in macrophage infiltration between TGF-β loaded biogel and healthy controls was not statistically significant.

### Biogel Enhances In Vivo Functional Recovery

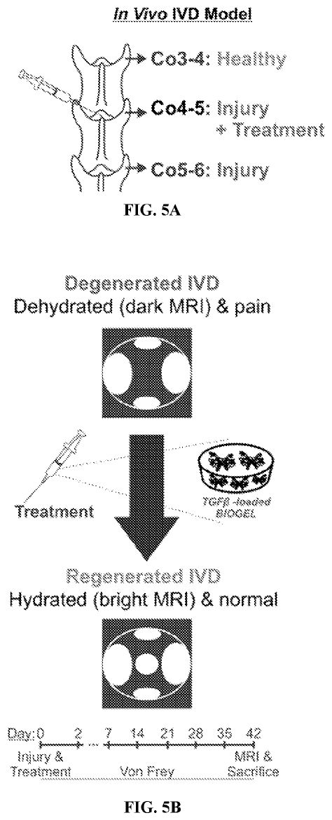

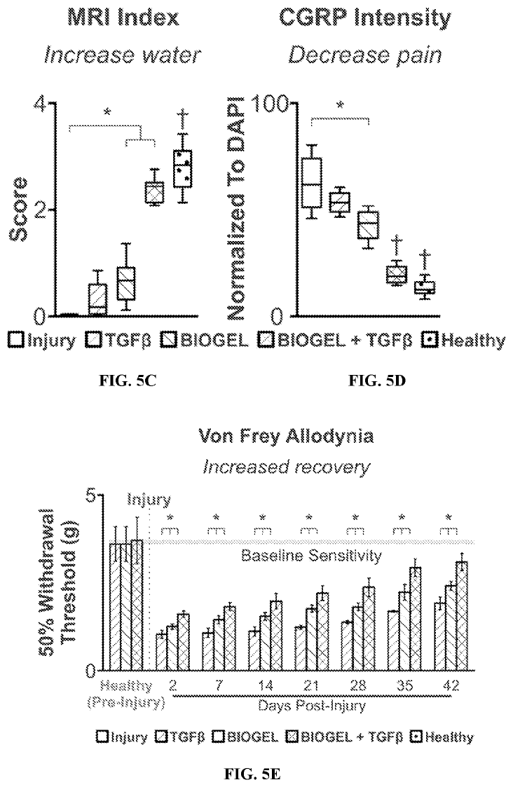

Functional recovery is the ultimate goal of treating IVD degeneration. For this, the regenerated IVD must have the load-bearing capacity of native IVD and an absence of IVD degeneration symptoms. The in vivo IVD degeneration model from was employed here to characterize functional recovery (FIG. 5A).

The IVD's capacity to retain water can be considered as the culmination of various factors, including the health of the cells and the degree of proteoglycan present [11, 40]. High water content is paramount to the viscoelastic properties of the IVD and effective load-bearing; progressive water loss indicates greater IVD degeneration (FIG. 5B) [11]. T2-weighted magnetic resonance imaging (T2-MRI) was employed to acquire coronal and axial images non-invasively to evaluate the water content of regenerated IVD on day 42 [40, 43]. Healthy control IVD appeared as a bright signal under T2-weighted MRI [11, 40, 43]. Conversely, injured IVD showed a significantly darker signal against the surrounding tissue, indicating severe degeneration and water loss. While TGF-β and bare biogel treatments showed T2-MRI images with moderate signal intensity at the center of the IVD, they exhibited significantly reduced area compared to the healthy control. In contrast, IVD treated with TGF-β loaded biogel resulted in the highest signal intensity and area out of the treatment cohorts, signifying the greatest water retention. When the T2-MRI images were graded, TGF-β was found to have negligible regeneration compared to the non-treated injury control (FIG. 5C). This is expected as the lost NP tissue from the nucleotomy is not replaced, and native cells must regenerate the tissue without a scaffold. Conversely, both biogel and TGF-β loaded biogel resulted in significant recovery of T2 contrast, with TGF-β loaded biogel exhibiting more extensive water retention. While bare biogel provided a well-hydrated scaffold for remodeling, it lacked the cell signaling cues to initiate remodeling towards cartilage-like tissue. TGF-β loaded biogel contained both cell signaling cues and a physical scaffold for tissue remodeling, resulting in the greatest regeneration.

One of the principal clinical manifestations of IVD degeneration is back pain, which is responsible for a significant portion of healthcare and economic burden (e.g., high medical expenses and lost productivity) in developed countries [44]. Reducing pain caused by IVD degeneration is a key step towards functional recovery as it will allow patients to resume normal activities after the IVD has regenerated damaged tissue. Neuropathic pain such as allodynia can be caused by IVD degeneration, so mechanical allodynia was measured to assess discogenic back pain recovery in IVD degeneration. Von Frey filaments were used to test allodynia by measuring paw withdrawal threshold and allodynia, while the presence of sensory neuropeptide Calcitonin Gene Receptor Protein (CGRP) was assessed via immunofluorescence (FIG. 5B) [45, 46]. Healthy IVD showed little staining for CGRP, while injured IVD showed abundant and intense CGRP. Neither TGF-β nor bare biogel treatments substantially reduced CGRP staining intensity (normalized to DAPI staining) (FIGS. 5C, 5D). Conversely, IVD treated with TGF-β loaded biogel showed significantly reduced CGRP staining intensity. These results indicate that encapsulating therapeutic payloads (e.g., TGF-β) in biogel potentiated discogenic pain reduction. As predicted by the greater histological recovery in the preceding sections, animals treated with TGF-β loaded biogel displayed a greater tolerance to mechanical forces (i.e., probing with Von Frey filaments) before initiating an adverse reaction (e.g., paw withdrawal) (FIG. 5E). This increased force/pain tolerance indicates less severe allodynia starting two days post-treatment, compared to animals treated with TGF-β or bare biogel.

The ability to treat IVD degeneration-related allodynia indicates accelerated recovery from discogenic back pain and earlier resumption of normal activity for human patients [44]. This expedited treatment of IVD symptoms would alleviate strain on healthcare and social security systems by presenting an effective treatment for one of the leading causes of medical disabilities in adults [44].

### Conclusions

In summary, provided is an injectable biogel system for enhanced IVD tissue regeneration. More specifically, tetrazine and norbornene functional groups were grafted onto gelatin to produce a biocompatible, biodegradable, and cell adhesive hydrogel that can crosslink in situ. The liquid GelTz and GelNb precursor solutions remained low viscosity across a wide range of temperatures, allowing for ergonomic injections and reducing shear stresses experienced by any encapsulated payloads. Once the liquid precursors were activated via simple mixing, a clinical operator was allowed a comfortable window of time to inject the solution (5-10 minutes) before it stabilized and formed a hydrogel that displayed biomechanical properties similar to healthy IVD tissue. To demonstrate synergy with existing growth factor therapies, the biogel system was supplemented with TGF-β to provide a sustained release profile, which potentiated the anti-inflammatory and pro-regenerative effects of TGF-β in vitro. Moreover, TGF-β loaded biogel was administered to an in vivo rat model of IVD degeneration to induce greater histological recovery (e.g., recovered tissue architecture and induced de novo matrix synthesis) and functional recovery (e.g., increased water retention and reduced discogenic pain) than either TGF-β or biogel component by itself. Since biogel relies on bioorthogonal chemistry and utilizes gelatin derived from collagen (a major component of mammalian extracellular matrices), this system can be easily extended to include other payloads, target other degenerative conditions, and regenerate various tissues. In short, given biogel's versatility in tissue engineering, this technology platform can be applicable to a broad range of regenerative medicine and clinical conditions other than IVD regeneration.

### References in Example 2

[1] M. A. Adams, P. J. Roughley, What is intervertebral disc degeneration, and what causes it?, Spine 31 (2006) 2151-2161
[2] R. D. Bowles, L. A. Setton, Biomaterials for intervertebral disc regeneration and repair, Biomaterials 129 (2017) 54-67
[3] A. J. Walsh, D. S. Bradford, J. C. Lotz, In vivo growth factor treatment of degenerated intervertebral discs, Spine 29 (2004) 156-163, https://doi.orQ/10.1097/01.BRS.0000107231.67854.9F.
[4] L. M. Wakefield, T. S. Winokur, R. S. Hollands, K. Christopherson, A. D. Levinson, M. B. Sporn, Recombinant latent transforming growth factor beta 1 has a longer plasma half-life in rats than active transforming growth factor beta 1, and a different tissue distribution, The Journal of clinical investigation 86 (1990) 1976-1984, https://doi.org/Doi 10.1172/Jci114932.
[5] K. Masuda, Biological repair of the degenerated intervertebral disc by the injection of growth factors, European Spine Journal 17 (2008) 441-451, https://doi.org/10.1007/s00586-008-0749-z.
[6] J. Li, D. J. Mooney, Designing hydrogels for controlled drug delivery, Nat. Rev. Mater. 1 (2016) 1-17, https://doi.org/10.1038/natrevmats.2016.71.
[7] J. M. Alonso, J. Andrade del Olmo, R. Perez Gonzalez, V. Saez-Martinez, Injectable hydrogels: From laboratory to industrialization, Polymers 13 (2021) 650, https://doi.org/10.3390/polym13040650.
[8] J. H. Lee, Injectable hydrogels delivering therapeutic agents for disease treatment and tissue engineering, Biomaterials research 22 (2018) 1-14, https://doi.org/10.1186/s40824-018-0138-6.
[9] N. Annabi, J. W. Nichol, X. Zhong, C. Ji, S. Koshy, A. Khademhosseini, F. Dehghani, Controlling the porosity and microarchitecture of hydrogels for tissue engineering, Tissue Engineering Part B: Reviews 16 (2010) 371-383, https://doi.org/10.1089/ten.TEB.2009.0639.
[10] S. T. Koshy, R. M. Desai, P. Joly, J. Li, R. K. Bagrodia, S. A. Lewin, N. S. Joshi, D. J. Mooney, Click-crosslinked injectable gelatin hydrogels, Adv. Healthcare Mater. 5 (2016) 541-547, https://doi.org/10.1002/adhm.201500757.
[11] B. V. Fearing, P. A. Hernandez, L. A. Setton, N. O. Chahine, Mechanotransduction and cell biomechanics of the intervertebral disc, JOR spine 1 (2018) e1026, https://doi.org/10.1002/jsp2.1026.
[12] R. F. Heary, N. Parvathreddy, S. Sampath, N. Agarwal, Elastic modulus in the selection of interbody implants, Journal of spine surgery 3 (2017) 163, https://doi.org/10.21037/iss.2017.05.01.
[13] C.-Y. Lin, H. Kang, J. P. Rouleau, S. J. Hollister, F. La Marca, Stress analysis of the interface between cervical vertebrae end plates and the Bryan, Prestige LP, and ProDisc-C cervical disc prostheses: an in vivo image-based finite element study, Spine 34 (2009) 1554-1560, https://doi.org/10.1097/BRS.0b013e3181aa643b.
[14] J. J. Rawlinson, K. P. Punga, K. L. Gunsallus, D. L. Bartel, T. M. Wright, Wear simulation of the ProDisc-L disc replacement using adaptive finite element analysis, Journal of Neurosurgery: Spine 7 (2007) 165-173, https://doi.org/10.3171/Spi-07/08/166.
[15] M. D. Harmon, D. M. Ramos, D. Nithyadevi, R. Bordett, S. Rudraiah, S. P. Nukavarapu, I. L. Moss, S. G. Kumbar, Growing a backbone-functional biomaterials and structures for intervertebral disc (ivd) repair and regeneration: challenges, innovations, and future directions, Biomaterials Science 8 (2020) 1216-1239, https://doi.org/10.1039/c9bm01288e.
[16] J. Stergar, L. Gradisnik, T. Velnar, U. Maver, Intervertebral disc tissue engineering: A brief review, Bosnian journal of basic medical sciences 19 (2019) 130, https://doi.orQ/10.17305/bibms.2019.3778.
[17] S. van Uden, J. Silva-Correia, J. M. Oliveira, R. L. Reis, Current strategies for treatment of intervertebral disc degeneration: substitution and regeneration possibilities, Biomaterials research 21 (2017) 1-19, https://doi.org/10.1186/s40824-017-0106-6.
[18] J. Zhu, R. E. Marchant, Design properties of hydrogel tissue-engineering scaffolds, Expert review of medical devices 8 (2011) 607-626, https://doi.org/10.1586/erd.11.27.
[19] J. Van Hoorick, L. Tytgat, A. Dobos, H. Ottevaere, J. Van Erps, H. Thienpont, A. Ovsianikov, P. Dubruel, S. Van Vlierberghe, (Photo-) crosslinkable gelatin derivatives for biofabrication applications, Acta Biomater. 97 (2019) 46-73, https://doi.org/10.1016/j.actbio.2019.07.035.
[20] A. B. Bello, D. Kim, D. Kim, H. Park, S.-H. Lee, Engineering and functionalization of gelatin biomaterials: From cell culture to medical applications, Tissue Engineering Part B: Reviews 26 (2020) 164-180
[21] D. M. Patterson, L. A. Nazarova, J. A. Prescher, Finding the right (bioorthogonal) chemistry, ACS Chem. Biol. 9 (2014) 592-605, https://doi.org/10.1021/cb400828a.
[22] A. Sivashanmugam, R. A. Kumar, M. V. Priya, S. V. Nair, R. Jayakumar, An overview of injectable polymeric hydrogels for tissue engineering, Eur. Polym. J. 72 (2015) 543-565, https://doi.org/10.1016/j.eurpolymj.2015.05.014.
[23] M. H. Amer, F. R. Rose, K. M. Shakesheff, M. Modo, L. J. White, Translational considerations in injectable cell-based therapeutics for neurological applications: concepts, progress and challenges, NPJ Regenerative medicine 2 (2017) 1-13, https://doi.org/10.1038/s41536-017-0028-x.
[24] K. Masuda, Y. Aota, C. Muehleman, Y. Imai, M. Okuma, E. J. Thonar, G. B. Andersson, H. S. An, A novel rabbit model of mild, reproducible disc degeneration by an anulus needle puncture: correlation between the degree of disc injury and radiological and histological appearances of disc degeneration, Spine 30 (2005) 5-14, https://doi.org/DOI 10.1097/01.brs.0000148152.04401.20.
[25] Y. Saruhashi, K. Mori, A. Katsuura, S. Takahashi, Y. Matsusue, S. Hukuda, Evaluation of standard nucleotomy for lumbar disc herniation using the Love method: results of follow-up studies after more than 10 years, European Spine Journal 13 (2004) 626-630, https://doi.org/10.1007/s00586-004-0690-8.
[26] J. Chin, K. Lang, Methods of incorporating an amino acid comprising a bcn group into a polypeptide using an orthogonal codon encoding it and an orthorgonal pylrs synthase, in: W.I.P. Organization (Ed.) Medical Research Council, 2013.
[27] D. P. Pioletti, Integration of mechanotransduction concepts in bone tissue engineering, Computer methods in biomechanics and biomedical engineering 16 (2013) 1050-1055, https://doi.org/10.1080/10255842.2013.780602.
[28] L. Cacopardo, N. Guazzelli, A. Ahluwalia, Characterizing and Engineering Biomimetic Materials for Viscoelastic Mechanotransduction Studies, Tissue Engineering Part B: Reviews (2021), https://doi.org/10.1089/ten.TEB.2021.0151.
[29] S. Umehara, S. Tadano, K. Abumi, K. Katagiri, K. Kaneda, T. Ukai, Effects of degeneration on the elastic modulus distribution in the lumbar intervertebral disc, Spine 21 (1996) 811-819, https://doi.orQ/10.1097/00007632-199604010-00007.
[30] J. C. Iatridis, L. A. Setton, M. Weidenbaum, V. C. Mow, Alterations in the mechanical behavior of the human lumbar nucleus pulposus with degeneration and aging, Journal of Orthopaedic Research 15 (1997) 318-322, https://doi.org/10.1002/jor.1100150224.
[31] E.-Y. Kim, B.-C. Kim, Lipopolysaccharide inhibits transforming growth factor-beta1-stimulated Smad6 expression by inducing phosphorylation of the linker region of Smad3 through a TLR4-IRAK1-ERK1/2 pathway, FEBS Lett. 585 (2011) 779-785, https://doi.org/10.1016/j.febslet.2011.01.044.
[32] W. Li, T. Liu, L. Wu, C. Chen, Z. Jia, X. Bai, D. Ruan, Blocking the function of inflammatory cytokines and mediators by using IL-10 and TGF-β: A potential biological immunotherapy for intervertebral disc degeneration in a beagle model, International journal of molecular sciences 15 (2014) 17270-17283
[33] H. Yang, C. Cao, C. Wu, C. Yuan, Q. Gu, Q. Shi, J. Zou, TGF-β1 suppresses inflammation in cell therapy for intervertebral disc degeneration, Sci. Rep. 5 (2015) 1-10
[34] N. E. Rajan, O. Bloom, R. Maidhof, N. Stetson, B. Sherry, M. Levine, N. O. Chahine, Toll-Like Receptor 4 (TLR4) expression and stimulation in a model of intervertebral disc inflammation and degeneration, Spine 38 (2013) 1343-1351
[35] T. Ohnishi, N. Iwasaki, H. Sudo, Causes of and molecular targets for the treatment of intervertebral disc degeneration: a review, Cells 11 (2022) 394
[36] K. Phillips, K. Cullen, N. Chiverton, A. Michael, A. Cole, L. Breakwell, G. Haddock, R. Bunning, A. Cross, C. Le Maitre, Potential roles of cytokines and chemokines in human intervertebral disc degeneration: interleukin-1 is a master regulator of catabolic processes, Osteoarthritis and Cartilage 23 (2015) 1165-1177, https://doi.org/10.1016/j.joca.2015.02.017.
[37] F.-J. Lyu, H. Cui, H. Pan, K. Mc Cheung, X. Cao, J. C. Iatridis, Z. Zheng, Painful intervertebral disc degeneration and inflammation: from laboratory evidence to clinical interventions, Bone Res. 9 (2021) 1-14, https://doi.org/10.1038/s41413-020-00125-x.
[38] P. J. McKeown-Longo, P. J. Higgins, Integration of canonical and noncanonical pathways in TLR4 signaling: complex regulation of the wound repair program, Advances in wound care 6 (2017) 320-329, https://doi.org/10.1089/wound.2017.0736.
[39] T. Kluba, T. Niemeyer, C. Gaissmaier, T. Grunder, Human anulus fibrosis and nucleus pulposus cells of the intervertebral disc: effect of degeneration and culture system on cell phenotype, Spine 30 (2005) 2743-2748, https://doi.ora/10.1097/01.brs.0000192204.89160.6d.
[40] S. Lim, S. B. An, M. Jung, H. P. Joshi, H. Kumar, C. Kim, S. Y. Song, J. R. Lee, M. Kang, I. Han, Local delivery of senolytic drug inhibits intervertebral disc degeneration and restores intervertebral disc structure, Adv. Healthcare Mater. 11 (2022) 2101483
[41] K. R. Nakazawa, B. A. Walter, D. M. Laudier, D. Krishnamoorthy, G. E. Mosley, K. L. Spiller, J. C. Iatridis, Accumulation and localization of macrophage phenotypes with human intervertebral disc degeneration, The Spine Journal 18 (2018) 343-356, https://doi.org/10.1016/j.spinee.2017.09.018.
[42] A. Kawakubo, K. Uchida, M. Miyagi, M. Nakawaki, M. Satoh, H. Sekiguchi, Y. Yokozeki, G. Inoue, M. Takaso, Investigation of resident and recruited macrophages following disc injury in mice, Journal of Orthopaedic Research 38 (2020) 1703-1709, https://doi.org/10.1002/jor.24590.
[43] C. W. Pfirrmann, A. Metzdorf, M. Zanetti, J. Hodler, N. Boos, Magnetic resonance classification of lumbar intervertebral disc degeneration, Spine 26 (2001) 1873-1878, https://doi.org/10.1097/00007632-200109010-00011.
[44] A. L. Dutmer, H. R. S. Preuper, R. Soer, S. Brouwer, U. Bultmann, P. U. Dijkstra, M. H. Coppes, P. Stegeman, E. Buskens, A. D. van Asselt, Personal and societal impact of low back pain: the Groningen spine cohort, Spine 44 (2019) E1443-E1451, https://doi.org/10.1097/BRS.0000000000003174.
[45] T. Ozawa, S. Ohtori, G. Inoue, Y. Aoki, H. Moriya, K. Takahashi, The degenerated lumbar intervertebral disc is innervated primarily by peptide-containing sensory nerve fibers in humans, Spine 31 (2006) 2418-2422
[46] S. R. Chaplan, F. W. Bach, J. Pogrel, J. Chung, T. Yaksh, Quantitative assessment of tactile allodynia in the rat paw, Journal of neuroscience methods 53 (1994) 55-63
[47] U. Y. Choi, H. P. Joshi, S. Payne, K. T. Kim, J. W. Kyung, H. Choi, M. J. Cooke, S. Y. Kwon, E. J. Roh, S. Sohn, M. S. Shoichet, I. Han, An Injectable Hyaluronan-Methylcellulose (HAMC) Hydrogel combined with Wharton's jelly-derived mesenchymal Stromal cells (WJ-MSCs) promotes degenerative disc repair, International journal of molecular sciences 21 (2020) 7391
[48] P. R. Mittl, J. P. Priestle, M. G. Gruetter, Human Transforming Growth Factor Beta 3, Crystallized from PEG 4000, 1996.
[49] P. R. Mittl, J. P. Priestle, D. A. Cox, G. Mcmaster, N. Cerletti, M. G. Grutter, The crystal structure of TGF-β3 and comparison to TGF-02: implications for receptor binding, Protein Sci. 5 (1996) 1261-1271
[50] R. Z. Kramer, J. Bella, P. Mayville, B. Brodsky, H. M. Berman, Sequence dependent conformational variations of collagen triple-helical structure, Nature structural biology 6 (1999) 454-457, https://doi.org/10.1038/8259.
[51] R. Z. Kramer, J. Bella, P. Mayville, B. Brodsky, H. M. Berman, Collagen, 1998.
[52] D. Sehnal, S. Bittrich, M. Deshpande, R. Svobodovi, K. Berka, V. Bazgier, S. Velankar, S. K. Burley, J. Koča, A. S. Rose, Mol* Viewer: modern web app for 3D visualization and analysis of large biomolecular structures, Nucleic Acids Res. 49 (2021) W431-W437, https://doi.org/10.1093/nar/gkab314.

### Example 3: Nanomaterial-Augmented, Injectable Hydrogel Promotes Neurological Recovery in Spinal Cord Injuries

Spinal cord injuries (SCI) are potentially life-altering conditions which can result in significant healthcare costs, decreased quality of life, and loss of motor function [1-3]. The poor prognosis after SCI is typically attributed to poor regenerative capacity of the central nervous system (CNS), which prevents the body from readily replacing damaged tissues and cells [1, 3]. Given the inability for higher mammals to induce robust CNS regeneration, biomedical interventions are required to assist patients with regaining lost neurological function. To restore neurological function after SCI, damaged neurons must be replaced [3]. While various stem cells can be expanded ex vivo and differentiate to theoretically form new replacement neurons (i.e., neurogenesis) at the SCI site, this process is obstructed by the SCI neuroinflammatory state [4]. Here, neuroinflammation presents itself as a persistently pro-neuroinflammatory environment that disrupts tissue repair, discourages neurogenesis, and limits functional recovery [3-5]. These detrimental features have impeded early attempts (e.g., dissociated cell suspensions and simple scaffolds) at regenerating SCI. Thus, subsequent attempts at targeting SCI regeneration must include mechanisms to allow stem cell neurogenesis despite a neuroinflammatory environment.

Among the most urgent concerns of SCI is the persistently high concentration of reactive oxygen species (ROS) [6]. These ROS induce significant cytotoxic effects and reduce the viability of both native and transplanted cells. To reduce the deleterious effects of ROS at the SCI site, manganese dioxide (MnO₂) nanosheets can be administered as an ROS scavenger [6]. Moreover, the process of ROS neutralization causes MnO₂ to biodegrade. Here, MnO₂ can act to limit cell death induced by ROS after SCI.

In addition to causing nonspecific cell death, SCI creates a hostile microenvironment which can limit neurogenesis. Therapeutic drugs can be employed to counter these anti-regenerative effects. The Notch signaling cascade is well-known to have (1) anti-inflammatory effects and (2) influence proliferation and differentiation (e.g., neurogenesis) in both embryonic and adult neural stem cells (NSC). While downregulation of Notch is effective at inducing neurogenesis in embryonic NSC, several reports have suggested that Notch downregulation in adult NSC may have context-dependent detrimental effects including NSC depletion. Thus, an embryonic NSC-like cell would be ideal to minimize the possibility of off-target effects from Notch downregulation. Human induced pluripotent stem cell-derived neuronal progenitor cells (hiPSC-NPC) have greater similarities to embryonic NSC compared to adult NSC while lacking the controversies found with embryonic-derived stem cells. Thus, drugs targeting Notch (e.g., DAPT) can be employed to restore the neurogenic potential of transplanted hiPSC-NPC in damaged spinal cords.

To minimize the potential of Notch downregulation off-target effects on local (adult) NSC, a controlled drug release mechanism must be used to localize DAPT to the lesion site. MnO₂ is well suited for this purpose since it readily biodegraded via ROS. This key degradation mechanism can be paired with high MnO₂-drug adsorption capacity to create a stimuli-responsive drug release mechanism to ameliorate SCI secondary injury. Here, payload-MnO₂ conjugates neutralize ROS present at the SCI site, which locally releases neurogenic and anti-inflammatory agents to inhibit further ROS production and induce neurogenesis in target hiPSC-NPC. Moreover, the strong physicochemical interactions between the MnO₂ carrier and DAPT payloads limits nonspecific diffusion away from the SCI site. To administer payload-MnO₂ conjugates at the SCI site, a suitable injectable carrier is required.

The ideal payload-MnO₂ delivery vehicle should also function to create a permissive environment for tissue regeneration. The recently reported injectable BIOGEL platform is a suitable candidate for these two target applications. The BIOGEL matrix is primarily composed of gelatin (i.e., denatured collagen), which can replace the extracellular matrix (ECM) damaged by the primary injury and various degenerative processes associated with secondary injury [8]. Additionally, gelatin matrices permit numerous cell types (including neurons) to form focal adhesions, infiltrate the hydrogel, and remodel the ECM as needed. This tissue regeneration platform is activated via a bioorthogonal tetrazine-norbornene crosslinking reaction to minimize interference with native biomolecules and host tissue [8]. Once the BIOGEL precursor solutions are mixed, an operator can inject the low-viscosity solution to the injury site before the hydrogel begins to set (~5 minutes post-mixing) [8]. This hydrogel injection procedure confers substantial benefits over transplanting pre-formed solid hydrogels as it avoids large surgical incisions that can exacerbate the SCI site [9]. Since the underlying bioorthogonal reaction is nonreactive toward MnO₂ and most drugs, BIOGEL is well situated to serve the dual role of delivering payload-MnO2 conjugates and promoting tissue regeneration.

Here, the inventors propose the use of a nanomaterial-augmented therapy to promote SCI recovery. This therapy consists of four components which integrate with each other to enhance regeneration. Human induced pluripotent stem cell-derived neuronal progenitor cells (hiPSC-NPC) differentiate into neurons to replace cells lost during SCI [1]. The ability for hiPSC-NPC to replace dead neurons is augmented by DAPT (γ-secretase inhibitor), which induces neurogenic and reduce pro-inflammatory pathways [10, 11]. While DAPT exhibits strong neuroprotective, neurogenic, and anti-inflammatory effects when administered to CNS injuries, its short half-life (approximately six hours) and hydrophobicity (requiring organic solvents) impedes its clinical applicability [10, 11]. Both limitations are addressed by adsorbing DAPT onto MnO₂, which imparts an extended drug release profile and enhances stability in aqueous environments. As an additional functionality, MnO₂ scavenges ROS to improve hiPSC-NPC transplantation viability. Since DAPT-MnO₂ cannot support cell growth in the absence of a solid matrix, BIOGEL will be used to (1) noninvasively deliver DAPT-MnO₂ and hiPSC-NPC to the SCI lesion site, (2) form a cell-permissive hydrogel that can support hiPSC-NPC during neurogenesis, and (3) replace damaged extracellular matrix within the injured spinal cord. With this integrative therapeutic design, key SCI regeneration requirements (e.g., replacing dead cells, inducing de novo generation of neurons, supplementing damaged SCI tissue) are addressed in a single, ergonomic treatment.

This proof-of-concept therapy was applied to in vitro and in vivo neuroinflammation and SCI models to demonstrate enhanced neurogenesis and recovery. hiPSC-NPC encapsulated in BIOGEL with DAPT-MnO₂ underwent more extensive neurogenesis and displayed more neuron-like morphologies than cells encapsulated with DAPT or MnO₂ alone. Additionally, immune cells co-cultured with DAPT-MnO₂—BIOGEL expressed fewer cytotoxic and pro-inflammatory cytokines. In vivo, mice with contused spinal cords exhibited reduced glial scarring, more extensive neuronal infiltration, enhanced cell survival, and reduced inflammation when treated with DAPT-MnO₂—BIOGEL. Since the key therapeutic design is highly modular (i.e., can readily substitute DAPT, MnO₂, and BIOGEL with other therapeutic drugs, nanomaterials, and injectable hydrogels) and applicable toward other neurological conditions including traumatic brain injuries, the nanomaterial-augmented injectable hydrogel described herein can pave the way to treat numerous CNS injuries.

### Results and Discussion

### Formation of DAPT-Manganese Dioxide Drug-Carrier Complexes

To address key deficiencies in pharmacological treatment of SCI (e.g., organic solvent vehicles and premature drug clearance), drug-manganese dioxide nanosheet (MnO₂) complexes were investigated. Small molecule drugs can be adsorbed onto MnO₂ via non-destructive physicochemical interactions to preserve the chemical structure of the pharmaceutical agents [7, 12]. In turn, MnO₂ acts as a water-stable carrier for hydrophobic drugs (removing the need for organic solvent vehicles) and biodegrades upon cellular activity [7, 12]. Moreover, the large drug-carrier complex limits the ability for the drug to diffuse away from the SCI lesion site [13]. As a proof-of-demonstration, the inventors adsorbed DAPT (neurogenic and anti-inflammatory drug) onto MnO₂ [10, 11].

DAPT-MnO₂ drug-carrier complexes were readily prepared in an aqueous-based overnight adsorption protocol. Hydrophobic DAPT adsorbed onto MnO₂ as confirmed by X-ray photoelectron spectroscopy (XPS). Fluorine peaks from DAPT can be viewed on the DAPT-MnO₂ spectrum, indicating successful adsorption of DAPT onto MnO₂ nanosheets. Moreover, the atomic ratio of fluorine to manganese indicates a loading capacity of 0.64 mg DAPT per mg Mn. This high drug loading capacity is advantageous for clinical use as it minimizes the DAPT-MnO₂ dosing required to achieve a therapeutic effect, thus minimizing the risk of excessive manganese causing manganism in the patient [14].

Since DAPT can adsorb onto MnO₂ nanosheets, excessive DAPT loading may induce multiple MnO₂ nanosheets to aggregate into multilayer assemblies, which may adversely affect colloidal stability [15]. To verify the absence of multilayer aggregates, transmission electron microscopy (TEM) was used to visualize MnO₂ nanosheets before and after DAPT adsorption. TEM images revealed no significant change in MnO₂ opacity upon DAPT adsorption, indicating that DAPT did not induce MnO₂ nanosheets to aggregate and become multilayer assemblies. To support this finding, (DAPT-)MnO₂ samples were probed with atomic force microscopy (AFM) to directly measure nanosheet thickness. While DAPT adsorption resulted in a small increase in MnO₂ height, the change was less than the thickness of a single MnO₂ nanosheet (i.e., less than 1 nm), further indicating a lack of multilayer aggregates. These results are supported by dynamic light scattering which show negligible change in hydrodynamic sizes after DAPT adsorption. One notable change after DAPT adsorption was a slight increase in Zeta potential towards a neutral charge with increasing DAPT loading. This result is likely due to neutral DAPT occupying the surface of the negatively charged MnO₂. Since excessive Zeta potential neutralization can compromise the aqueous stability of drug-carrier complexes, subsequent DAPT-MnO₂ adsorption was fixed to an initial loading concentration to 1:3 w/w DAPT:MnO₂ [15]. Here, it was demonstrated DAPT-MnO₂ complexes retain the enhanced aqueous stability of MnO₂ nanosheets for use in biological systems.

Once DAPT-MnO₂ complexes are formed, the solutions can be mixed with BIOGEL precursors and dehydrated to form shelf-stable solids. This processing scheme removes any trace organic solvents used in the DAPT-MnO₂ complex synthesis procedure and ensures high biocompatibility when injected into patients. Moreover, these dehydrated DAPT-MnO₂—BIOGEL precursor samples can be reconstituted in aqueous solutions without causing severe DAPT-MnO₂ aggregation [16]. In fact, BIOGEL precursors appear to stabilize DAPT-MnO₂ in PBS compared to bare (DAPT-)MnO₂ which experienced severe aggregation when resuspended in saline or media [16]. The drug-MnO₂—BIOGEL precursor preparation workflow represents a significant advancement in pharmaceutical treatment of SCI since it is highly modular (i.e., can substitute DAPT and MnO₂ with other payloads and nanomaterials), creates a large drug-carrier conjugate to limit diffusion and premature clearance from the SCI site, and stabilizes hydrophobic drugs in aqueous media.

Supplementing BIOGEL with Manganese Dioxide

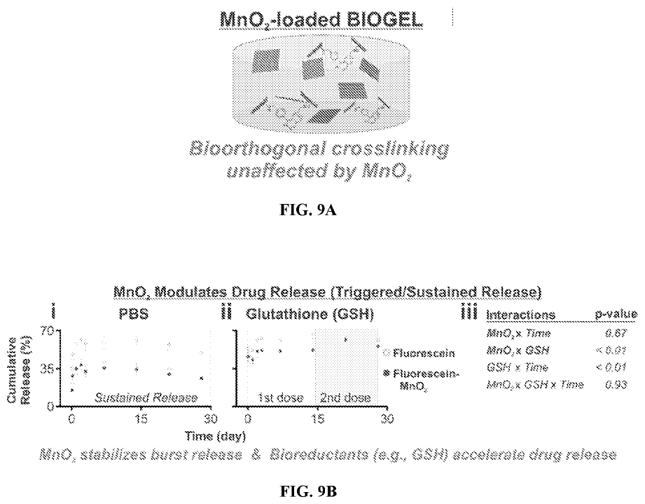

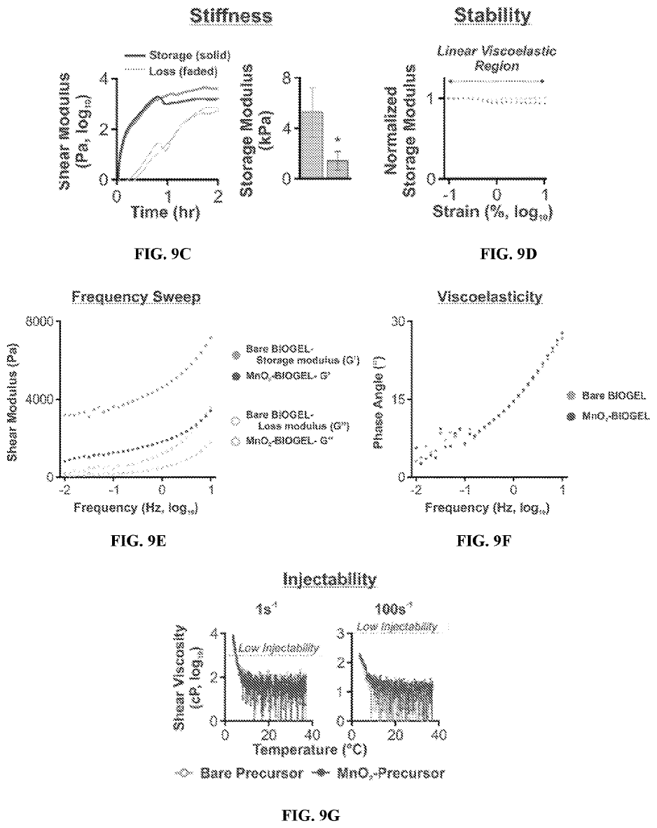

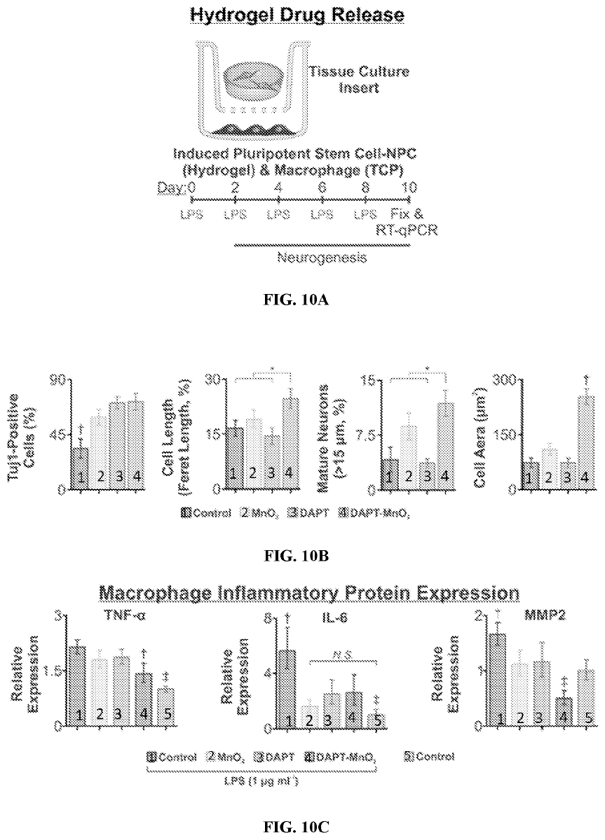

In addition to modulating the stability and physicochemical properties of DAPT, MnO₂ was selected to supplement the BIOGEL (gelatin tetrazine-norbornene) injectable hydrogel platform. This nanomaterial was chosen due to its ability to (1) modulate drug release profiles from BIOGEL hydrogels and (2) scavenge reactive oxygen species that are released as a part of SCI secondary injury [12]. Moreover, the bioorthogonal crosslinking mechanism of tetrazine-norbornene ligation minimizes the risk of DAPT-MnO₂ cross-reactivity with BIOGEL precursors during crosslinking (FIG. 9A). Preliminary experiments revealed that MnO₂ does not appreciably interfere with BIOGEL crosslinking until over 0.6 mg ml⁻¹ of MnO₂ is included in the mixed precursor solution (data not shown). To ensure consistent crosslinking, drug-MnO₂ conjugate loading was restricted to 0.3 mg ml⁻¹, which is under the cytotoxic MnO₂ concentration threshold for human induced pluripotent stem cell-derived neuronal progenitor cell (hiPSC-NPC).

To demonstrate the free radical scavenging capacity of MnO₂, BIOGEL samples with and without encapsulated MnO₂ were challenged with a hydrogen peroxide (H₂O₂, source of cytotoxic free radicals) [6]. Bare and MnO₂—BIOGEL samples were incubated in an H₂O₂ solution to permit diffusion throughout the hydrogel and subsequently assayed with xylenol orange-iron to stain for peroxide penetration. While bare BIOGEL shows significant H₂O₂ content throughout the entire hydrogel (as indicated by the strong purple color), MnO₂—BIOGEL exhibits significantly reduced staining around its periphery and retains the characteristic brown coloration of intact, unreacted MnO₂ in its center. These results indicate that MnO₂ scavenged and neutralized free radicals around the periphery of the BIOGEL sample, where H₂O₂ diffusion and concentration is highest. Moreover, the MnO₂—BIOGEL center was spared from peroxide and free radical diffusion. For subsequent in vitro studies, MnO2 can be employed to effectively neutralize cytotoxic free radicals before significant cellular and tissue damage can occur.

To assess the ability for MnO₂ to moderate drug release profiles, fluorescein-MnO2 complexes were encapsulated in BIOGEL samples for fluorescence measurements in phosphate buffered saline (PBS) (FIG. 9B). As a control, free fluorescein was also encapsulated in BIOGEL to compare release of free drug from the hydrogel. Throughout the entire 28-day test period, significantly less drug release was detected from fluorescein-MnO₂ BIOGEL samples compared to free (non-adsorbed) fluorescein-BIOGEL samples (FIG. 9B). This result may be due to the large fluorescein-MnO₂ complexes displaying significantly lower diffusivity through the hydrogel, resulting in greater fluorescein retention over time [13]. In addition, the BIOGEL samples exhibit a sustained release profile after a 3-day initial burst release profile. Thus, drug-MnO₂ complexes can be incorporated into BIOGEL to moderate drug burst release, retain a greater proportion of loaded drugs within the hydrogel (i.e., reduce premature clearance), and promote sustained release.

In addition to modulating drug burst release, MnO₂ can be used to impart stimuli-responsive drug release in physiological media [12]. This enhanced drug release control is due to MnO₂ biodegadation via bioreductants secreted by cells [12]. To characterize the release of drugs in response to cellular activity, free fluorescein and fluorescein-MnO₂ BIOGEL samples were incubated in PBS supplemented with glutathione (GSH, cell-secreted bioreductant in cerebrospinal fluid) (FIG. 9B) [17]. While free fluorescein samples displayed similar release profiles with and without glutathione, fluorescein-MnO2 samples exhibited vastly different drug release behaviors when exposed to glutathione. In the presence of glutathione, fluorescein-MnO₂ exhibits significantly higher drug release. This accelerated drug release is likely due to the degradation of the MnO₂ nanosheet carrier [12]. Without the nanocarrier restricting fluorescein diffusion through the hydrogel, a greater amount of fluorescein is released from the BIOGEL sample [13]. Moreover, this release is dose dependent as evidenced by (1) a sustained release after exposure to the initial GSH dose and (2) additional burst release upon the addition of a second dose of GSH. Taken together, MnO₂ demonstrates ample ability to moderate drug release from BIOGEL as evidenced by three-way ANOVA analysis indicating a significant interaction between MnO₂ and GSH (FIG. 9B). With the inclusion of MnO₂ in BIOGEL, drug burst release is reduced, and encapsulated payloads are not liberated until MnO₂ is degraded by bioreductants.

The inclusion of nanomaterials into hydrogels has been documented to have profound effects on the final composite scaffold biomechanical properties. Such changes in hydrogel properties can affect cellular behavior, influence the mechanical forces on surrounding tissue (i.e., stress shielding), and hydrogel robustness after implantation [18, 19]. At a MnO₂ loading concentration of 0.3 mg ml⁻¹, BIOGEL samples experienced a significant reduction in shear modulus (FIG. 9C). This change in stiffness is advantageous for SCI regeneration as numerous reports indicate neurogenesis to be accelerated in softer hydrogels [18]. While hydrogel stiffness changed with the inclusion of MnO₂, the onset of hydrogel crosslinking (i.e., approximately 5 minutes) and stiffness plateau (i.e., approximately 1.5 hours) remains similar. In addition, the viscoelastic behavior of BIOGEL appears unaffected by MnO₂ (FIG. 9E, 9F). When examining bare and MnO₂-loaded BIOGEL under significant strain (up to 10%), both hydrogels remain within the linear viscoelastic region, whereby neither hydrogel undergoes yielding or structural breakdown (FIG. 9D). Thus, MnO₂ (0.3 mg ml⁻¹) can be incorporated into BIOGEL precursor solutions to fine-tune the final hydrogel stiffness without affecting its stability under strain.

While MnO₂ imparts significant advantages in scavenging ROS, controlling drug release, and modulating hydrogel stiffness, it is prudent to verify that MnO₂ does not impair the clinical translation of BIOGEL treatments. The BIOGEL precursor solutions were designed to remain low viscosity (e.g., under 10³ cP) for a wide range of temperature to facilitate injection in a clinical environment [8]. Moreover, lower viscosities reduce the shear stresses experienced by suspended cells and payloads during injection [9]. With the inclusion of 0.3 mg ml⁻¹ MnO₂, there is no significant changes in the BIOGEL precursor solution viscosity across the entire range of temperatures investigated (FIG. 9G). In other words, MnO₂ does not interfere with the ergonomic injection of BIOGEL precursors into patients.

Here, the inventors demonstrated that MnO₂ can be successfully incorporated into BIOGEL for enhanced physicochemical function (i.e., ROS scavenging, controlled drug release, and modulating hydrogel stiffness) without compromising the clinical translatability of the injectable hydrogel system nor the mechanical stability of the resulting composite hydrogel. Since these enhanced physicochemical functions are applicable for halting neuroinflammation and inducing neurogenesis, the MnO₂—BIOGEL platform holds significant promise for SCI treatment.

### Drug-MnO₂ Complexes Enhance Stem Cell Differentiation and Anti-Inflammatory Effects in BIOGEL

Though stem cell therapies have been attempted to replace lost neurons and restore neurological function in SCI patients, transplanted cells often fail to differentiate and integrate into the host neurological processes [1]. The lackluster progress during in vivo stem cell therapies can be attributed to a complex cascade of multiple maladaptive processes (e.g., tissue damage after the mechanical trauma of the SCI). Among the notable injury pathways, neuroinflammation has been determined to be one of the most persistent barriers to neurological and functional recovery [1, 3, 5]. To address the need for more effective stem cell therapies, DAPT (Notch pathway inhibitor, neurogenesis promoter and anti-inflammatory drug) was included in the MnO₂—BIOGEL platform to mitigate the effects of neuroinflammation [10, 11].

To assess the in vitro efficacy of DAPT-loaded MnO₂—BIOGEL for treating neuroinflammation and SCI, a cell culture insert-based setup was established to examine neurogenesis under a neuroinflammatory environment. THP-1-derived macrophages were induced into a pro-inflammatory state with lipopolysaccharide (LPS) in the well plate, while hiPSC-NPC were encapsulated in various BIOGEL therapies and incubated in cell culture inserts [5, 10]. The extent of neuroinflammation was determined in macrophages via reverse transcriptase-quantitative polymerase chain reaction (RT-qPCR) for key inflammation genes tumor necrosis factor α (TNF( ), interleukin-6 (IL-6), and chemokine (C-C motif) ligand 5 (CCL5) [5]. Concurrently, neurogenesis marker expression was determined in hiPSC-NPC via whole mount immunocytochemistry staining and image analysis for (1) percent of Class III β-tubulin (Tuj1), (2) percent of neuron-like cells (cells with Tuj1 staining surface area longer than >15 μm), and (3) average cell area [20, 21]. These quantitative metrics will provide insight into which experimental conditions are most suited to promoting functional neurological recovery in vivo.

Without MnO₂ or DAPT loading, hiPSC-NPC encapsulated in BIOGEL undergo minimal neurogenesis as evidenced by relatively few elongated Tuj1⁺ cells. Rather, most cells remain spherical with negligible Tuj1 staining, indicating that the pro-inflammatory environment induced by chronically inflamed immune cells (i.e., LPS-stimulated macrophages) severely diminished the ability for stem cells to differentiate and replenish SCI-damaged neurons. The inclusion of DAPT moderately stimulated neurogenesis marker expression, indicating moderate bioactivity. This pharmacological payload was further potentiated when adsorbed onto MnO₂ as evidenced by the majority of cells becoming Tuj1⁺ with a substantial proportion exhibiting neuron-like morphologies and greater cell areas. Even in the absence of macrophages or inflammatory factors (i.e., non-neuroinflammatory environment), hiPSC-NPC encapsulated in BIOGEL with DAPT-MnO₂ exhibited more neuron-like morphology compared to cells encapsulated with DAPT or MnO₂ alone. These results indicate that neurogenesis marker expression can be optimized by co-encapsulating the cells with DAPT-MnO₂ in BIOGEL.

Macrophages co-cultured with bare BIOGEL exhibited typical pro-inflammation gene expression patterns known to interfere with neurogenesis [5]. Depending on the specific gene, DAPT inclusion in BIOGEL produced either negligible to moderate reduction in TNFα, IL-6, and CCL5 expression. These payloads exhibit significantly greater bioactivity when are adsorbed onto MnO₂. DAPT adsorbed on MnO₂ was particularly effective at suppressing the expression of pro-inflammatory genes. This is presumably due to MnO₂ decreasing burst release and enabling a more sustained release profile for DAPT. Together with the immunocytochemistry staining results, adsorbing DAPT onto MnO₂ significantly increases in vitro stem cell neurogenesis efficiency while simultaneously decreasing the neuroinflammatory environment which normally diminishes in vivo stem cell neurogenesis after SCI.

### DAPT-MnO₂ in BIOGEL Enhances In Vivo Histological Recovery

Due to the complex nature of the nervous system, in vivo animal experiments are required to gain further insight before human clinical trials. To mimic the most common modes of SCI in developed nations (e.g., car crashes, falls), a moderate contusion SCI model was chosen using the RWD Impactor device at T9-T10 [2, 3]. Since contusion injuries generally leave the dura intact, BIOGEL can be directly injected into the spinal cord without the need to further perturbate the sensitive injury site [2, 3, 9]. At predetermined post-injury times, the mice were sacrificed for tissue harvesting and assessment of injury regeneration. More specifically, two interconnected regenerative processes were explored during the in vivo experiment: neurogenesis and microenvironment modulation. While injured spinal cords display low neurogenesis because of a hostile and pro-inflammatory microenvironment, an ideally regenerated spinal cord permits high rates of neurogenesis (to restore neurological function) due to a more favorable microenvironment. These two processes were assayed to determine the extent of SCI.

Neurogenesis is vital to replace damaged neurons and restore neurological connectivity across the SCI lesion site [4, 11, 21]. Here, DAPT induces neurogenesis in hiPSC-NPC by inhibiting Notch signaling and expression (FIG. 11A) [11]. The result is a reduction in neural stem and neuronal progenitor cell marker expression (Sox2 and Nestin expression, FIG. 11A and -iii) concurrent with an increase in neurons (MAP2 expression, FIG. 11A) [4, 11, 21]. While DAPT-MnO₂—BIOGEL treatment induced statistically significant changes in only Notch3 and Sox2, there was a notable trend toward greater neurogenesis across all genes tested compared to bare BIOGEL. Thus, there is strong in vivo support for DAPT-MnO₂—BIOGEL treatment to regenerate lost neurons.

To potentiate neurogenesis and integrate newly formed neurons into the host tissue, the spinal cord microenvironment must be modulated away from a neuroinflammatory state [5]. Three key regenerative processes affected by neuroinflammation are reactive gliosis (GFAP, FIG. 11B), pro-/anti-inflammation dysregulation (IL-1β and IL-10, FIG. 11B), and proliferation (Ki67, FIG. 11B) [4, 5]. DAPT-MnO₂—BIOGEL treatment induced significant downregulation of maladaptive reactive gliosis and pro-inflammation cytokine expression while bolstering pro-regenerative anti-inflammatory cytokine expression and proliferation. With these key post-SCI processes modulated towards repair, DAPT-MnO₂—BIOGEL treatment can provide a suitable microenvironment for neurogenesis.

### Conclusion

In summary, an injectable bioorthogonal hydrogel (BIOGEL) was augmented with drug-nanomaterial conjugates to enhance its neurogenic and anti-inflammatory capacity. Here, DAPT (hydrophobic neurogenic and anti-inflammatory drug) was adsorbed onto MnO₂ to form a water-stable, cell-responsive drug-carrier complex. The resulting DAPT-MnO₂ was loaded into BIOGEL without compromising the injectability or mechanical integrity of the final hydrogel. DAPT-MnO₂-loaded BIOGEL displayed greater neurogenic and anti-inflammatory activity in an in vitro spinal cord injury model compared to DAPT and MnO₂ individually loaded into BIOGEL. This synergistic DAPT-MnO₂ complex was extended to in vivo use, where mice treated with DAPT-MnO₂—BIOGEL displayed greater neurogenesis marker expression and reduced neuroinflammation after contusion SCI. Here, the applicability of BIOGEL was expanded with drug-nanomaterial conjugates for enhanced pharmacokinetic control and tissue regeneration. The presented drug-carrier-hydrogel platform is highly modular (i.e., different components can be integrated with minimal difficulty), meaning it can be reconfigured to target and treat other disease states. Since MnO₂—BIOGEL can be combined with a large variety of pharmaceutical agents to accelerate tissue regeneration, this technology should be applicable across numerous biomedical applications.

### Experimental Methods

### Materials

SYBR Green PCR Master Mix was purchased from Applied Biosystems. (2S)—N-[(3,5-Difluorophenyl)acetyl]-L-alanyl-2-phenyl] glycine 1,1-dimethylethyl ester (DAPT) and phorbol 12-myristate 13-acetate (PMA) were purchased from Bio-Techne. Dulbecco's Modified Eagle Medium/Nutrient Mixture F-12 (DMEM/F-12) and Matrigel® were purchased from Corning. Anhydrous ethanol was purchased from Decan Labs. Carbon tape was purchased from Electron Microscope Sciences. Dimethylformamide (DMF) and hydrogen peroxide were purchased from Fisher Scientific. Accutase was purchased from Innovative Cell Technologies. DNA primers were purchased from Integrated DNA Technologies. DNase I was purchased from New England Biolabs. Silicon wafers were purchased from Omniscience. Atomic force microscopy cantilevers (14 μm height, <7 nm radius, 160 μm length, 26 N/m, 300 kHz) were purchased from NanoAndMore USA. Parafilm was purchased from Pechiney Plastic Packaging. Recombinant human FGF-basic (bFGF) was purchased from PeproTech. Nuclease-free water was purchased from Qiagen. Anhydrous dimethylsulfoxide (DMSO), chloroform, fluorescein, neutral buffered formalin solution, 2-propanol, tissue culture-treated 24-well plates, tissue culture-treated 96-well plates, and Triton X-100 were purchased from Sigma-Aldrich. Carbon tape, mica discs, and stainless-steel discs were purchased from Ted Pella. β-mercaptoethanol in Dulbecco's phosphate buffered saline (DPBS), B27™ Supplement, DPBS, fetal bovine serum, glycogen, lipopolysaccharide (LPS), N2™ Supplement, Neurobasal™ media, normal goat serum, Pierce™ Quantitative Peroxide Assay Kit, PrestoBlue™ Cell Viability Reagent, RPMI 1640, SuperScript III First-Strand Synthesis System, and TRIzol was purchased from Thermo Fisher. Cell culture inserts (0.4 μm pore size, polycarbonate, 6.5 mm) were purchased from VWR. BIOGEL precursors were synthesized as reported elsewhere [8].

### (GFP-)hiPSC-NPC and THP-1 Cell Culture

(GFP-)hiPSC-NPC were maintained using 0.5% B27, 0.5% N2, 20 ng ml⁻¹ bFGF in 1:1 Neurobasal: DMEM/F-12 proliferation media on Matrigel®-coated tissue culture plastic in a humidified, 37° C. incubator with 5% carbon dioxide. Cell passaging was performed using Accutase. Genotypic stability was ensured by culturing both cell lines for at least 20 population doublings and at least two cryopreservation cycles using 10% DMSO in proliferation media.

THP-1 were maintained using 10% FBS, 55 μM β-mercaptoethanol in RPMI 1640 on tissue culture plastic in a humidified, 37° C. incubator with 5% carbon dioxide.

DAPT-MnO₂ Cytotoxicity Assay for hiPSC-NPC

For MnO₂ cytotoxicity, a 96-well plate was coated with Matrigel® prior to seeding with hiPSC-NPC (20,000 cells, 0.125 ml per well) suspended in proliferation media. Cells were allowed to stabilize overnight. Various stock MnO₂ solutions (8.5×) were prepared in distilled water and directly administered to hiPSC-NPC in proliferation media (e.g., 16.7 μL of 8.5 mg ml⁻¹ stock MnO₂ solution was prepared to test 1 mg ml⁻¹ MnO₂).

For DAPT, MnO₂, and DAPT-MnO₂ cytotoxicity, cell seeding was slightly modified (5,000 cells, 0.200 ml per well). MnO₂ (0.3 mg ml⁻¹ in distilled water, 1 μL), DAPT (0.1 mg ml⁻¹ in DMF, 1 μL), and DAPT-MnO₂ (0.3 mg ml⁻¹, 1 μL) solutions were added to test wells.

hiPSC-NPC were allowed to incubate with their respective test solutions for 24 hours. Cytotoxicity was assayed using PrestoBlue™ Cell Viability Reagent. Cells were washed with DPBS twice to remove excess payload before PrestoBlue™ in proliferation media (10% w/w, 150 μl) was added. The assay was allowed to develop in a humidified, 37° C. incubator before being read on a microwell plate reader (Tecan) according to manufacturer specification.

### Reverse Transcriptase-Quantitative Polymerase Chain Reaction (RT-qPCR)

For in vitro RT-qPCR characterization, THP-1 cells were lysed with TRIzol Reagent (1 ml) on day 10 of neuroinflammatory co-culture.

For in vivo RT-qPCR characterization, homogenized spinal cord segments were centrifuged (12,000 g for 10 minutes at 4° C.) to separate solubilized RNA from solid tissue matter.

RNA was isolated from TRIzol via chloroform (200 μL) extraction and glycogen in 2-propanol (50 μg mL⁻¹, 500 μL) precipitation at 4° C. The resulting crude RNA was collected via centrifugation and washed with aqueous ethanol (75% v/v, 1 mL) before resuspension in nuclease-free water (15 μL). The concentration and purity were quantified with a Nanodrop Lite Spectrophotometer (Thermo Fisher) before an aliquot of crude RNA (1 μg) was digested using DNase I (to remove residual genomic DNA according to manufacturers' specification). The purified RNA solution was converted to cDNA using SuperScript III First Strand Synthesis System (according to manufacturer specification) with the aid of a Mastercycler ep Gradient S (Eppendorf) thermal cycler. Oligo(dT)₂₀ primers were used to selectively convert mRNA to cDNA.

qPCR was performed using SYBR Green PCR Master Mix on a StepOnePlus™ Real-Time PCR System (Applied Biosystems) with ΔΔC_{T} quantitation method. Primers are listed below (Table 3). ΔΔC_{T} values were subjected to one-way ANOVA followed by Tukey's HSD post-hoc analysis to determine statistical significance.

TABLE 3
RT-qPCR primer sequences

| Gene | Forward | Reverse |
|---|---|---|
| Name | Primer (5′-3′) | Primer (5′-3′) |
| Notch3 | TGC CAG AGT TCA | CAC AGG CAA ATC |
| | GTG GTG G | GGC CAT C |
| | (SEQ ID NO: 13) | (SEQ ID NO: 14) |
| Sox2 | GCG GAG TGG AAA | GGG AAG CGT GTA |
| | CTT TTG TCC | CTT ATC CTT CT |
| | (SEQ ID NO: 15) | (SEQ ID NO: 16) |
| Nestin | CCC TGA AGT CGA | CTG CTG CAC CTC |
| | GGA GCT G | TAA GCG A |
| | (SEQ ID NO: 17) | (SEQ ID NO: 18) |
| MAP2 | GCC AGC CTC GGA | GCT CAG CGA ATG |
| | ACA AAC A | AGG AAG GA |
| | (SEQ ID NO: 19) | (SEQ ID NO: 20) |
| GFAP | CGG AGA CGC ATC | AGG GAG TGG AGG |
| | ACC TCT G | AGT CAT TCG |
| | (SEQ ID NO: 21) | (SEQ ID NO: 22) |
| IL-1β | CTT CAA ATC TCA | CCA CGG GAA AGA |
| | CAG CAG CAG CAC | CAC AGG TAG |
| | ATC | (SEQ ID NO: 24) |
| | (SEQ ID NO: 23) | |
| IL-10 | GCT CTT ACT GAC | CGC AGC TCT AGG |
| | TGG CAT GAG | AGC ATG TG |
| | (SEQ ID NO: 25) | (SEQ ID NO: 26) |
| Ki67 | ATC ATT GAC CGC | GCT CGC CTT GAT |
| | TCC TTT AGG T | GGT TCC T |
| | (SEQ ID NO: 27) | (SEQ ID NO: 28) |
| GAPDH | ATG ACA TCA AGA | CAT ACC AGG AAA |
| | AGG TGG TG | TGA GCT TG |
| | (SEQ ID NO: 29) | (SEQ ID NO: 30) |

### In Vitro Neuroinflammatory Characterization (Whole Mount Immunohistology Staining)

On day 10 of neuroinflammatory co-culture, hiPSC-NPC in BIOGEL were washed twice with Dulbecco's phosphate buffered saline (DPBS) before overnight fixation in neutral buffered formalin solution (10%) at 4° C. Excess formalin was removed, and BIOGEL samples were washed twice with DPBS.

hiPSC-NPC in BIOGEL were subjected to a serial de-/rehydration using (1) 50% ethanol in DPBS, (2) 80% ethanol in distilled water, (3) 100% ethanol, (4) 20% DMSO in ethanol, (5) 80% ethanol in distilled water, (6) 50% ethanol in DPBS, (7) DPBS, and (8) 2% Triton X-100 in DPBS (2 ml, 15 minute, 4° C. per step) before overnight incubation in blocking buffer (5% v/v normal goat serum and 0.3% Triton X-100 in DPBS, 2 ml, 4° C.). Samples were placed on a rotary shaker during incubation to promote diffusion throughout the BIOGEL matrix.

Excess blocking buffer was removed before overnight incubation with primary antibodies (see Table 4) in ADB (1% w/v bovine serum albumin and 0.3% Triton X-100 in DPBS, 0.5 ml, 4° C.). BIOGEL samples were washed with DPBS overnight (1 ml, 4° C.) twice to remove excess primary antibodies before incubation with secondary antibodies in ADB overnight (0.5 ml, 4° C.) protected from light. BIOGEL samples were washed with DPBS overnight (1 ml, 4° C.) twice to remove excess secondary antibodies and stored in DPBS (4° C.) protected from light.

TABLE 4
In Vivo BIOGEL Injection Sites

| # | Sagittal Coordinate from SCI Epicenter | Transverse Coordinate from SCI Epicenter |
|---|---|---|
| Injection | (+: rostral, −: caudal) | (+: right, −: left) |
| 1 | +1 mm | +1 mm |
| 2 | −1 mm | +1 mm |
| 3 | −1 mm | −1 mm |
| 4 | +1 mm | −1 mm |

Whole mount samples were imaged with an epifluorescent microscope (Nikon). Z-stack images were reconstructed into a projection image and processed with ImageJ.

### In Vivo Contusion SCI Characterization (Paraffin Section Immunohistology Staining)

OCT-embedded spinal cords were sectioned (12 μm) using a cryostat (Thermo Scientific) and mounted on charged glass slides. Primary antibody, secondary antibody, and nuclear staining solutions were prepared in cryosection blocking buffer 0.05% Triton X-100, 2% donkey serum, 3% bovine serum albumin in PBS).

Spinal cord sections were immersed in methanol (10 minute) before treatment with primary antibodies (overnight, 4° C.), secondary antibodies (1 hour, ambient), and DAPI (1 hour, ambient).

PBS washing (3×10 minutes) was performed in between each step. Stained spinal cord sections were sealed with Cytoseal 60 as per manufacturer specification.

BIOGEL precursors GelTz and GelNb were synthesized as previously reported [8]. In addition, MnO₂ nanosheets were synthesized as reported in the literature [12], the content of which is incorporated in its entirety.

### Drug-MnO₂—BIOGEL Preparation

Stock solutions were made by dissolving DAPT in dimethylformamide (1 mg ml⁻¹), MnO₂ in distilled water (3 mg ml⁻¹), fluorescein in dimethylformamide (1 mg ml⁻¹), and GelTz in distilled water (5% w/v).

Stock DAPT, MnO₂, and/or fluorescein solution were mixed with deionized water (DAPT and MnO₂: 10% v/v, fluorescein: 2% v/v payload solution), covered with aluminum foil, and placed on a tube rotator overnight. After overnight mixing, stock GelTz solution was added to each payload solution (1.67% w/v GelTz). The payload-GelTz solutions were placed back on the tube rotator for overnight mixing before vacuum concentration to produce a fibrous solid (stored at −20° C. until point of use).

### AFM, DLS, TEM, XPS, and Zeta Potential Sample Preparation

AFM samples were prepared by drop-casting (DAPT-)MnO₂ solutions (30 μg ml⁻¹ MnO₂ basis, 20 μL) onto mica discs. The coated substrates were placed on a spin-coater set to 2000 rpm for 1 minute, mounted onto stainless-steel discs with the aid of carbon tape, and allowed to dry overnight before imaging using a Cypher VRS Atomic Force Microscope (Oxford Instruments).

(DAPT-)MnO₂ solutions were diluted to 30 μg ml⁻¹ (MnO₂ basis) prior to filling spectrophotometer cuvettes and folded capillary cells for dynamic light scattering and Zeta potential measurements with a ZetaSizer Nano NS (Malvern).

TEM samples were prepared by drop-casting (DAPT-)MnO₂ solutions (30 μg ml⁻¹ MnO₂ basis, 3 μL) onto copper TEM grids. The solutions were allowed to dry overnight before imaging using a Philips CM12 TEM (FEI).

Silicon wafers were cleaned via successive ethanol, water, and Triton X-100 in water (1% v/v) washings in a sonicator bath. XPS samples were prepared by drop-casting DAPT (1 mg ml⁻¹, 10 μL), MnO₂, and DAPT-MnO₂ solutions (0.3 mg ml⁻¹ MnO₂ basis, 10 μL) onto cleaned silica wafers. The samples were allowed to dry under vacuum before measurement using a K-Alpha X-Ray Photoelectron Spectrophotometer (Thermo Fisher).

### Hydrogen Peroxide Neutralization

Dried (MnO₂)—GelTz and GelNb samples were reconstituted in DPBS (5% w/v). An equivalent volume of (MnO₂)-GelTz and GelNb solution were mixed and pipetted into a cylindrical 0.5 cm diameter, 0.8 cm height Parafilm divot (50 μL mixed solution per divot) within 5 minutes of mixing. Mixed BIOGEL precursor solutions were allowed to crosslink for 15 minutes at room temperature.

The crosslinked BIOGEL samples were then transferred into a centrifuge tube containing excess hydrogen peroxide in DPBS (100 μM H₂02, 50 ml). BIOGEL in H₂O₂ was allowed to incubate at room temperature on a tube rotator for 30 minutes. BIOGEL samples were retrieved, carefully pipetted to remove excess H₂02, and incubated with Peroxide Working Reagent (0.5 ml, Pierce™ Quantitative Peroxide Assay Kit) for 15 minutes according to manufacturer specification.

### Fluorescein Model Drug Release

Dried fluorescein(-MnO₂)-GelTz samples were reconstituted in DPBS (5% w/v). An equivalent volume of fluorescein(-MnO₂)-GelTz and GelNb solution were mixed and pipetted into a cylindrical 0.5 cm diameter, 0.8 cm height Parafilm divot (50 μL mixed solution per divot) within 5 minutes of mixing. BIOGEL precursor solutions were allowed to crosslink for 15 minutes at room temperature.

The crosslinked BIOGEL samples were then transferred into a 24-well plate with DPBS (1.5 ml per well) and incubated at 37° C. on a rotary shaker (40 rpm). Parafilm was used to seal the well plate edges and reduce evaporation. At predetermined times (i.e., 1 hour, 4 hr, 1 day, 3 d, 7 d, 14 d, 21 d, 28 d), aliquots (50 μl) were taken from each well and diluted with DPBS (300 μl) for spectroscopic assessment on a microwell plate reader (Tecan, λₑₓ=488 nm, λₑₘ, =525 nm).

### Rheometry and Viscometry

Rheology and viscometry measurements were conducted using a Kinexus Ultra rotational rheometer (Malvern) equipped with two flat stainless-steel plates (top geometry 20 mm diameter). Solutions (200 μL) were loaded into a 0.6 mm gap prior to data collection.

Bare BIOGEL and MnO₂—BIOGEL hydrogel crosslinking was characterized via oscillometry at a constant 37° C., 1 Hz, 1% shear strain for two hours. (MnO₂)—GelTz and GelNb solutions (5% w/v in PBS) were mixed immediately prior to loading onto the rheometer.

Crosslinked BIOGEL samples were characterized via the following oscillometry tests: (1) frequency sweep from 0.01 to 10 Hz (constant 37° C., 1% shear strain) and (2) amplitude sweep from 0.1 to 10% shear strain (constant 37° C., 1 Hz frequency).

Bare GelTz and MnO₂—GelTz hydrogel precursor injectability was characterized via viscometry in a temperature sweep from 37° C. to 4° C. (cooling rate 1° C. per minute) at 1 and 100 s-1 shear rates.

### In Vitro Neuroinflammatory Co-Culture

A 24-well plate were seeded with THP-1 (100,000 cell, 0.5 ml per well) in proliferation media supplemented with 50 ng ml-1 phorbol 12-myristate-13-acetate (PMA). Cells were allowed to adhere and differentiate into macrophages for 3 days before media was replaced with (PMA absent) proliferation media for overnight culture.

To initiate neuroinflammation (Day 0), THP-1 proliferation media was replaced with hiPSC-NPC proliferation media (1 ml) supplemented with lipopolysaccharide (1 μg ml⁻¹, LPS). THP-1-derived macrophages were incubated in the LPS-supplemented media for 4 hours before the media was replaced with (LPS absent) hiPSC-NPC proliferation media (1 ml).

Meanwhile, hiPSC-NPC were dissociated using Accutase. Excess media was removed via centrifugation, and hiPSC-NPC were resuspended in GelNb precursor solution (10 mil ml⁻¹ cells, 5% w/v GelNb in DPBS). The cell-GelNb solution was mixed with an equivalent volume of payload-GelTz precursor solution (5% w/v GelTz in DPBS) and pipetted into a cylindrical 0.5 cm diameter, 0.8 cm height Parafilm divot (50 μL mixed solution per divot) within 5 minutes of mixing. BIOGEL precursor solutions were allowed to crosslink for 15 minutes at room temperature before transfer into cell culture inserts with hiPSC-NPC proliferation media (200 μL) and placement into THP-1-seeded wells.

To initiate hiPSC-NPC differentiation and simulate chronic neuroinflammation (Day 2, 4, 6, and 8), THP-1 were restimulated with LPS (1 μg ml-1) in hiPSC-NPC neurogenesis media (proliferation media without bFGF) for 4 hours. During this time, BIOGEL cell culture inserts and THP-1 conditioned media were transferred to a fresh 24-well plate. After the 4-hour restimulation period, the BIOGEL cell culture inserts were placed back in the THP-1-seeded wells for continued culture.

After 10 days of co-culture, hiPSC-NPC in BIOGEL and THP-1 were harvested for whole mount immunocytochemistry staining and RT-qPCR, respectively. Primers and immunostaining antibodies are listed in Tables S1 and S2, respectively.

### Animal Model of Contusion SCI and Treatment

All animal study procedures were approved by the Institutional Animal Care and Use Committee (IACUC) of Rutgers University (PROTO999900038). 8- to 12-week-old female C57BL/6 mice (18-22 g) were kept in a controlled environment (22±1° C., 50±1% relative humidity, and 12/12 hr light/dark cycle) for the duration of the study. Animals were anesthetized by isoflurane inhalation (1-5%) before being placed on heating pads for the duration of surgery and until recovered from anesthesia. Additional analgesics buprenorphine (3.25 mg/kg) and bupivacaine (2 mg/kg) were administered subcutaneously. The eyes were lubricated with ophthalmic ointment to prevent corneal drying. The surgical area (thoracic vertebrae T8-T12) was prepared by shaving the dorsum of the mouse followed by sterilization with betadine and 70% ethanol. A skin incision was made longitudinally (~1.5 cm) over the laminectomy site to expose the fascia covering T8-T12. After removing the fascia, a pair of micro-scissors were used to remove the vertebral processes covering the spinal cord. Once the spinal cord is exposed and surrounding bone is removed, the mouse was placed on a stereotactic frame and placed under an RWD Precision Impactor (RWD Life Science). A 10-g metal probe was propelled at 1.6 m/s [dwell time (xx)] was used to induce a moderate contusion injury.

After injury, the mouse was transferred to a stereotactic injector. Four injections (see Table Sxx) were performed using a 26-gauge Hamilton needle and syringe. Each injection administered 0.5 μl of mixed bare or DAPT-MnO₂ BIOGEL solution at 0.4- and 0.8-mm depths (total 1 μl) at a rate of 0.2 μl/min. After confirming no BIOGEL leakage, a piece of subcutaneous fat was placed on top of the contusion site before the fascia was closed using Nylon sutures. The surgical site was further closed with stainless steel wound clips.

Mice were transferred into single-housing cages maintained with heating pads until normal activity level resumed. Cephazolin (25-50 mg/kg) and saline (0.5 ml) were subcutaneously injected daily and twice-daily, respectively. Additionally, assisted bladder voiding was performed twice daily.

### In Vivo Contusion SCI Characterization

Seven days after contusion SCI and treatment, animals were sacrificed for tissue harvest.

For gene expression, animals were anesthetized with isoflurane before euthanasia via decapitation. A 4 mm segment of spinal cord tissue (centered at the SCI lesion site) was immediately removed and placed in TRIzol. The tissue was homogenized via needle shearing (20 G needle) before RT-qPCR.

For immunostaining, animals were anesthetized with isoflurane before euthanasia via transcardiac puncture and saline/paraformaldehyde perfusion. The spinal cord was removed, immersed in sucrose (30%) overnight, and embedded in OCT compound before sectioning and immunostaining.

### Statistical Analysis

Statistical testing was conducted using R (statistical program) or OriginLab. All data are visualized as mean±standard deviation.

### Computer Graphics

OriginLab was used for data graphing unless indicated otherwise. Blender and ChemDraw were used to generate select graphics. Select graphics were created from BioRender.com.

### References in Example 3

[1] S. Liu, T. Schackel, N. Weidner, R. Puttagunta, Biomaterial-supported cell transplantation treatments for spinal cord injury: challenges and perspectives, Frontiers in cellular neuroscience 11 (2018) 430, https://doi.org/10.3389/fncel.2017.00430.
[2] S. Saadoun, N. D. Jeffery, Acute traumatic spinal cord injury in humans, dogs, and other mammals: the under-appreciated role of the dura, Frontiers in neurology 12 (2021) 629445, https://doi.org/10.3389/fneur.2021.629445.
[3] M. D. Norenberg, J. Smith, A. Marcillo, The pathology of human spinal cord injury: defining the problems, Journal of neurotrauma 21 (2004) 429-440, https://doi.orQ/10.1089/089771504323004575.
[4] E. A. Gilbert, N. Lakshman, K. S. Lau, C. M. Morshead, Regulating Endogenous Neural Stem Cell Activation to Promote Spinal Cord Injury Repair, Cells 11 (2022) 846, https://doi.org/10.3390/cells11050846.
[5] D. Kempuraj, R. Thangavel, P. Natteru, G. Selvakumar, D. Saeed, H. Zahoor, S. Zaheer, S.

Iyer, A. Zaheer, Neuroinflammation induces neurodegeneration, Journal of neurology, neurosurgery, and spine 1 (2016)
[6] Z. Jia, H. Zhu, J. Li, X. Wang, H. Misra, Y. Li, Oxidative stress in spinal cord injury and antioxidant-based intervention, Spinal cord 50 (2012) 264-274, https://doi.org/10.1038/sc.2011.111.
[7] G. Dey, L. Yang, K.-B. Lee, L. Wang, Characterizing molecular adsorption on biodegradable MnO₂ nanoscaffolds, The Journal of Physical Chemistry C 122 (2018) 29017-29027, https://doi.org/10.1021/acs.jpcc.8b09562.
[8] J. Luo, A. Darai, T. Pongkulapa, B. Conley, L. Yang, I. Han, K.-B. Lee, Injectable bioorthogonal hydrogel (BIOGEL) accelerates tissue regeneration in degenerated intervertebral discs, Bioactive Materials 23 (2023) 551-562, https://doi.org/https://doi.org/10.1016/j.bioactmat.2022.11.017.
[9] M. H. Amer, F. R. Rose, K. M. Shakesheff, M. Modo, L. J. White, Translational considerations in injectable cell-based therapeutics for neurological applications: concepts, progress and challenges, NPJ Regenerative medicine 2 (2017) 1-13, https://doi.org/10.1038/s41536-017-0028-x.
[10] P.-N. Tsao, S.-C. Wei, M.-T. Huang, M.-C. Lee, H.-C. Chou, C.-Y. Chen, W.-S. Hsieh, Lipopolysaccharide-induced Notch signaling activation through JNK-dependent pathway regulates inflammatory response, Journal of biomedical science 18 (2011) 1-9, https://doi.org/10.1186/1423-0127-18-56.
[11] L. Borghese, D. Dolezalova, T. Opitz, S. Haupt, A. Leinhaas, B. Steinfarz, P. Koch, F. Edenhofer, A. Hampl, O. Brustle, Inhibition of notch signaling in human embryonic stem cell-derived neural stem cells delays G1/S phase transition and accelerates neuronal differentiation in vitro and in vivo, Stem cells 28 (2010) 955-964, https://doi.org/10.1002/stem.408.
[12] J. Luo, L. Yang, S.-T. Dean Chueng, B. Conley, C. Rathnam, K.-B. Lee, Advanced Drug Delivery Modulation via Hybrid Nanofibers Enhances Stem Cell Differentiation, ACS Applied Materials Interfaces 14 (2022) 34488-34501, https://doi.orQ/10.1021/acsami.2c10288.
[13] C. A. Grabowski, A. Mukhopadhyay, Size effect of nanoparticle diffusion in a polymer melt, Macromolecules 47 (2014) 7238-7242, https://doi.org/10.1021/ma501670u.
[14] K. M. Andruska, B. A. Racette, Neuromythology of manganism, Current epidemiology reports 2 (2015) 143-148, https://doi.org/10.1007/s40471-015-0040-x.
[15] F. Gambinossi, S. E. Mylon, J. K. Ferri, Aggregation kinetics and colloidal stability of functionalized nanoparticles, Advances in colloid interface science 222 (2015) 332-349, https://doi.org/10.1016/j.cis.2014.07.015.
[16] X. Huangfu, J. Jiang, J. Ma, Y. Liu, J. Yang, Aggregation kinetics of manganese dioxide colloids in aqueous solution: influence of humic substances and biomacromolecules, Environmental science technology 47 (2013) 10285-10292, https://doi.org/10.1021/es4003247.
[17] H. Bayir, V. E. Kagan, Y. Y. Tyurina, V. Tyurin, R. A. Ruppel, P. D. Adelson, S. H. Graham, K. Janesko, R. S. Clark, P. M. Kochanek, Assessment of antioxidant reserves and oxidative stress in cerebrospinal fluid after severe traumatic brain injury in infants and children, Pediatric research 51 (2002) 571-578, https://doi.org/10.1203/00006450-200205000-00005.
[18] M. S. Ting, J. Travas-Sejdic, J. Malmstrom, Modulation of hydrogel stiffness by external stimuli: soft materials for mechanotransduction studies, Journal of Materials Chemistry B 9 (2021) 7578-7596, https://doi.org/10.1039/d1tb01415c.
[19] E. Eisenbarth, Biomaterials for tissue engineering, Adv. Eng. Mater. 9 (2007) 1051-1060, https://doi.org/10.1002/adem.200700287.
[20] C. Compagnucci, F. Piemonte, A. Sferra, E. Piermarini, E. Bertini, The cytoskeletal arrangements necessary to neurogenesis, Oncotarget 7 (2016) 19414, https://doi.org/10.18632/oncotarget.6838.
[21] A. Kumar, V. Pareek, M. A. Faiq, P. Kumar, C. Kumari, H. N. Singh, S. K. Ghosh, Transcriptomic analysis of the signature of neurogenesis in human hippocampus suggests restricted progenitor cell progression post-childhood, IBRO reports 9 (2020) 224-232, https://doi.org/10.1016/j.ibror.2020.08.003.

### Example 4: Engineered Neuronal Progenitor Cell-Derived Exosomes Ameliorate Traumatic Brain Injury

Traumatic Brain Injury (TBI) is a significant public health challenge that causes over 2 million emergency room visits and collectively costs billions of dollars annually. TBI begins with a sudden blow, jolt, or penetrating head injury, termed primary injury, that causes significant tissue damage. The damage then initiates neuroinflammatory pathways, called secondary injury, in which the surrounding tissue yields to high oxidative stress, axonal degeneration, and immune cell activation. Prolonged exposure from secondary injury results in widespread cell death of surrounding viable cells that increases the injury lesion area. Secondary injury also hinders regeneration for months following impact by the continuous upregulation of pro-inflammatory and neurotoxic cytokines. Curtailing inflammation reduces tissue damage sustained during secondary injury. Many therapeutic moieties have been identified as reducing inflammation, ranging from small molecules to genes and antibodies. However, the therapy's efficacy depends widely on multi-day delivery to the target tissue. Targeting neural tissue multiple times requires the therapeutic to intrinsically pass the Blood-Brain Barrier (BBB) and reach the injury lesion. Poor BBB penetration can significantly limit a therapeutic's drug efficacy.

Biomaterial-based approaches aid in neural tissue remodeling. During TBI, the ECM matrix becomes severely disordered and prevents regeneration. Activated astrocytes release chondroitin sulfate proteoglycans (CSPGs) that directly inhibit neurogenesis (Brain Research, 2023). CSPG release also correlates with upregulated levels of collagen, laminin, and fibronectin, creating a dense environment prohibiting neurogenesis. Upregulation of these ECM biomolecules also cause dysfunction for many cell signaling pathways vital for neuronal regeneration, including the Rho/Rho-associated protein kinase (ROCK), focal adhesion kinase (FAK), and the PI3K/Akt signaling pathways. These signaling pathways can be restored with hydrogels. Hydrogels restore the ECM by mimicking the biophysical properties of neural tissue. Gelatin-based hydrogels inhibit CSPG synthesis to reduce ECM density, which encourages neuronal regeneration. By mimicking neural tissue, hydrogels can stop the activation of dysfunctional pathways and synthesis of proteoglycans to increase neuronal regeneration and reduce glial scarring.

Exosomes have emerged in recent years as a powerful therapeutic moiety to treat CNS disease and injuries. Exosomes produced under hypoxic conditions are particularly potent in facilitating wound regeneration. Deferoxamine (DFO) stabilizes the HIF-1a complex, which then localizes to the nucleus to activate several growth factor pathways, including VEGF. VEGF promotes angiogenesis and reduces the ischemia plaguing the injury site that is also hindering neural tissue regeneration. Incubating DFO with NPCs also upregulates miRNA-9 to control neural stem progenitor cell fate through transcription factor silencing and upregulation. Notable transcription pathways include FoxO transcription factor family, nuclear receptor TLX, and Res1 silencing transcription family (REST). The exosomes composing miRNA-9 and VEGF, represent a dual strategy to promote neuronal differentiation and angiogenesis. Both factors facilitate wound healing of the injury site.

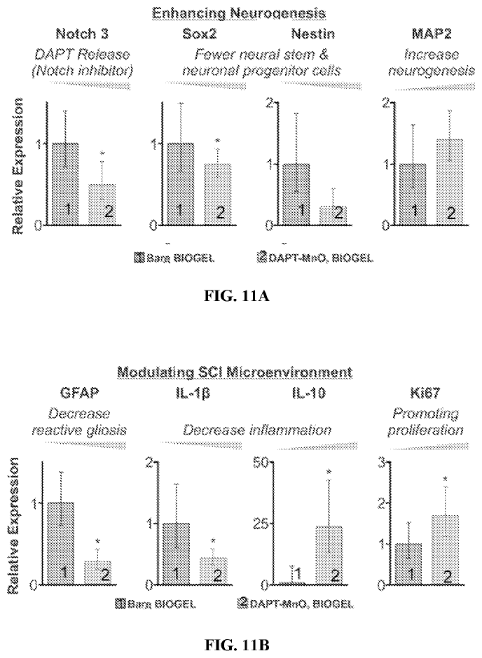

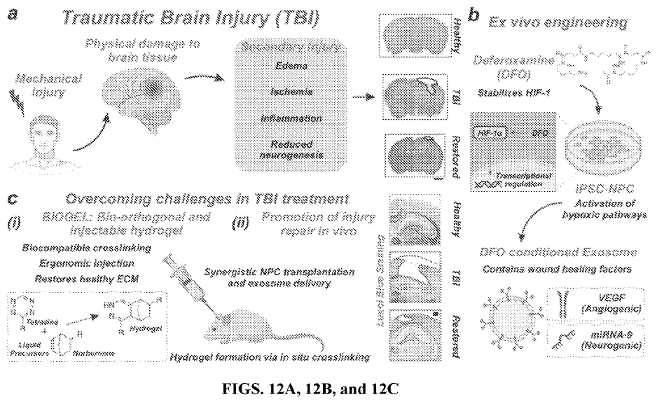

To address these limitations, the above-described hydrogel (BIOGEL) was combined with exosomes containing miRNA-9 and VEGF for traumatic brain injury (FIGS. 12A-12C). Biogel is biocompatible, delivers therapeutic moieties over multiple days, and reduces invasive surgery. Biogel was prepared by mixing gelatin-tetrazine and gelatin-norbornene liquid precursors. After 5 minutes, the liquid precursors underwent a bio-orthogonal reaction between the tetrazine and the norbornene to form the hydrogel. The exosomes were then prepared via ex vivo engineering of iPSC-NPCs with deferoxamine (DFO) to mimic hypoxic conditions. The exosomes and iPSC-NPCs were then co-transplanted inside Biogel for a TBI rat model via right craniotomy. The neurogenic and pro-angiogenic potential of the exosomes were investigated.

### Results and Discussion

### Pro-angiogenic Exosome Isolation and Biogel Synthesis

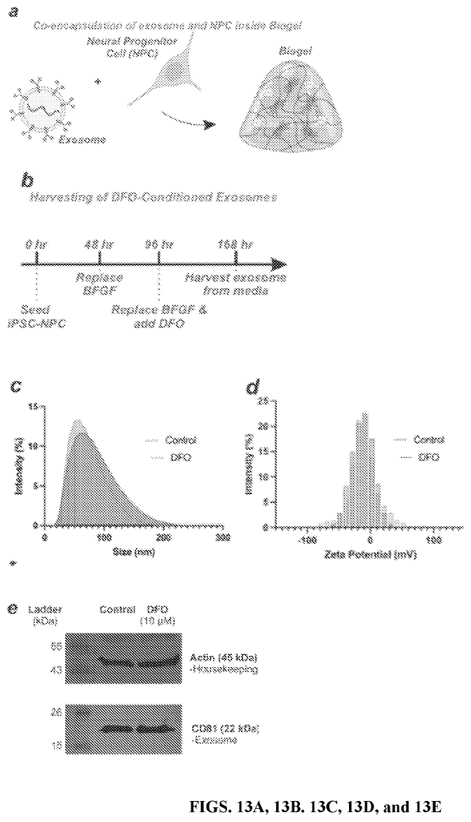

miRNA-9 containing exosomes were created from Induced Pluripotent Stem Cells-Neural Progenitor Cells (iPSC-NPCs). To upregulate miRNA-9 expression, cells were stimulated with Deferoxamine (DFO) to mimic hypoxic conditions. During this time, hypoxia-inducible factor 1-alpha expression increases. This upregulation leads to significant CNS tissue regeneration—as reported elsewhere. Following DFO incubation, the media was collected and exosome isolation was performed (FIG. 13B). Nanoparticle tracking analysis showed an even size distribution between 20-200 nm (FIG. 13C). The exosomes were also co-spotted with uranyl acetate onto a TEM grid. The uranyl acetate bound to all phospholipids of the exosome lipid membrane, making the nanoparticles white spheres (FIG. 13D). Western blot identified the exosome marker CD81 (FIG. 13E). With all of this characterization data, the correct isolation of exosomes from iPSC-NPCs following DFO incubation was confirmed. Next, the co-encapsulated the isolated exosomes with iPSC-NPCs inside the Biogel platform to form the Biogel-Exosome (DFO) platform.

Furthermore, the platform supports sustained exosome release, which is integral since delivery of miRNA-9 has been shown to reduce the therapeutic effect dramatically. The data shows the isolation of DFO-conditioned exosomes that contain a correct size distribution, which NTA and TEM confirm, and the correct housekeeping markers. The correct mechanical properties for CNS tissue regeneration were shown by the hydrogel mimicking the viscosity of CNS tissue. The Biogel+Exo (DFO) significantly decreased lesion area, encouraged neurogenesis and neuronal differentiation, protected oligodendrocytes, and increased remyelination. Thus, the Biogel+Exo (DFO) platform led to significant neural tissue regeneration.

### Experimental Methods

### BIOGEL-Exosome Preparation and Encapsulation

Dried GelTz and GelNb samples were reconstituted in DPBS (5% and 10% w/v, respectively). GelNb solution was diluted with an appropriate amount of exosome solution to reach the target exosome concentration and a final concentration of 5% w/v GelNb.

Hydrogels were formed by mixing an equal volume of GelTz and GelNb solution (total 50 μL) and pipetting into a cylindrical 0.5 cm diameter, 0.8 cm height parafilm divot within 5 minutes of mixing. Mixed BIOGEL precursor solutions were allowed to crosslink for 15 minutes under ambient temperatures.

### Exosome Release Profile

Dried fluorescein(-MnO₂)-GelTz samples were reconstituted in DPBS (5% w/v). An equivalent volume of fluorescein(-MnO₂)-GelTz and GelNb solution were mixed and pipetted into a cylindrical 0.5 cm diameter, 0.8 cm height Parafilm divot (50 μL mixed solution per divot) within 5 minutes of mixing. BIOGEL precursor solutions were allowed to crosslink for 15 minutes at room temperature.

The crosslinked BIOGEL samples were then transferred into a 24-well plate with DPBS (1.5 ml per well) and incubated at 37° C. on a rotary shaker (40 rpm). Parafilm was used to seal the well plate edges and reduce evaporation. At predetermined times (i.e., 1 hour, 4 hr, 1 day, 3 d, 7 d, 14 d, 21 d, 28 d), aliquots (50 μl) were taken from each well and diluted with DPBS (300 μl) for spectroscopic assessment on a microwell plate reader (Tecan, λex=488 nm, λem=525 nm).

### Rheometry and Viscometry

Rheology and viscometry measurements were conducted using a Kinexus Ultra rotational rheometer (Malvern) equipped with two flat stainless-steel plates (top geometry 20 mm diameter). Solutions (200 μL) were loaded into a 0.6 mm gap prior to data collection.

Bare BIOGEL and MnO₂—BIOGEL hydrogel crosslinking was characterized via oscillometry at a constant 37° C., 1 Hz, 1% shear strain for two hours. (MnO₂)—GelTz and GelNb solutions (5% w/v in PBS) were mixed immediately prior to loading onto the rheometer.

Crosslinked BIOGEL samples were characterized via the following oscillometry tests: (1) frequency sweep from 0.01 to 10 Hz (constant 37° C., 1% shear strain) and (2) amplitude sweep from 0.1 to 10% shear strain (constant 37° C., 1 Hz frequency).

Bare GelTz and MnO₂-GelTz hydrogel precursor injectability was characterized via viscometry in a temperature sweep from 37 to 4° C. (cooling rate 1° C. per minute) at 1 and 100 s-1 shear rates.

### In Vitro Neuroinflammatory Co-Culture

A 24-well plate was seeded with THP-1 (100,000 cell, 0.5 ml per well) in proliferation media supplemented with phorbol 12-myristate-13-acetate (PMA, 50 ng ml-1). Cells were allowed to adhere and differentiate into macrophages for 3 days before media was replaced with (PMA absent) proliferation media for overnight culture.

To initiate neuroinflammation (Day 0), THP-1 proliferation media was replaced with hiPSC-NPC proliferation media (1 ml) supplemented with lipopolysaccharide (1 μg ml-1, LPS).

THP-1-derived macrophages were incubated in the LPS-supplemented media for 4 hours before the media was replaced with (LPS absent) hiPSC-NPC proliferation media (1 ml). Meanwhile, hiPSC-NPC were dissociated using Accutase. Excess media was removed via centrifugation, and hiPSC-NPC were resuspended in GelNb precursor solution (10 mil ml-1 cells, 5% w/v GelNb in DPBS). The cell-GelNb solution was mixed with an equivalent volume of payload-GelTz precursor solution (5% w/v GelTz in DPBS) and pipetted into a cylindrical 0.5 cm diameter, 0.8 cm height Parafilm divot (50 μL mixed solution per divot) within 5 minutes of mixing. BIOGEL precursor solutions were allowed to crosslink for 15 minutes at room temperature before transfer into cell culture inserts with hiPSC-NPC proliferation media (200 μL) and placement into THP-1-seeded wells.

To initiate hiPSC-NPC differentiation and simulate chronic neuroinflammation (Day 2, 4, 6, and 8), THP-1 were restimulated with LPS (1 μg ml-1) in hiPSC-NPC neurogenesis media (proliferation media without bFGF) for 4 hours. During this time, BIOGEL cell culture inserts and THP-1 conditioned media were transferred to a fresh 24-well plate. After the 4-hour restimulation period, the BIOGEL cell culture inserts were placed back in the THP-1-seeded wells for continued culture.

After 10 days of co-culture, hiPSC-NPC in BIOGEL and THP-1 were harvested for whole mount immunocytochemistry staining and RT-qPCR, respectively.

### Animal Model of Cortical TBI and Treatment

All animal study procedures were approved by the Institutional Animal Care and Use Committee (IACUC) of CHA Bundang Medical Center (IACUC200141).

10-week-old female Sprague Dawley rats were kept in a controlled environment (22±1° C., 50±1 0% relative humidity, and 12/12 hr light/dark cycle) for the duration of the study. Animals were anesthetized by intraperitoneal injection of Zoletil® (50 mg kg-1) and Rompun® (10 mg kg-1) before being placed on heated pads for the duration of surgery. The surgical area was prepared by shaving the dorsum of the skull followed by sterilization with 70% ethanol and povidone iodine. A midline skin incision was made to expose the right parietal bone for craniotomy via a diamond bur drill. Once the brain was exposed, the rat was placed on a stereotactic frame and positioned under an RWD Precision Impactor (RWD Life Science). A 5 mm cylindrical hammer was propelled at 4 m s-1 to a depth of 3 mm (dwell time 5 s) to induce a mild/moderate cortical TBI. The surgery site was closed using Nylon sutures.

Rats were transferred into single-housing cages maintained with heating pads until normal activity level resumed. For 3 days post-surgery, animals were prophylactically treated with Cefazolin and Ketoprofen.

One week after TBI, the surgical site was reopened to expose original injury site. Any fluid buildup in the cystic lesion site was aspirated. Exosome and mixed BIOGEL-exosome solutions were prepared and administered to the TBI lesion site (20 μL). The surgery site was closed using Nylon sutures.

Rats were returned to their single-housing cages and recovery proceeded as described for the TBI. Additionally, Cefazolin and Ketoprofen was resumed for 3 days post-surgery.

### Histological Recovery Assessment of Cortical TBI Regeneration

Animals were sacrificed six weeks post-treatment to evaluate tissue structure and morphology. Brains were fixed in neutral buffered formalin (10%) for one week, paraffin-embedded, sliced using a microtome (Leica) to produce coronal sections (10 μm), dewaxed, and rehydrated.

Tissue architecture was determined by Hematoxylin & Eosin staining. Sections were mounted using mounting media and scanned with a microscope (OLYMPUS). Lesion volume was determined using built-in ImageJ functions and blinded reviewers.

Immunohistochemistry staining was performed. Sectioned samples were incubated with primary antibodies at 4° C. for overnight, washed with DPBS and Tween 20, incubated with secondary antibodies at room temperature for one hour, washed, and counter-stained with DAPI for 10 minutes. Stained sections were mounted using mounting media and scanned with a fluorescence microscope (Zeiss). Positive staining areas were determined using built-in ImageJ functions.

### Statistical Analysis

Statistical testing was conducted using R (statistical program) or OriginLab except for in vivo data, which was analyzed using GraphPad Prism. All data were visualized as mean: standard deviation. Statistical significance was defined as p-value<0.05 when analyzed by One-Way ANOVA followed by Tukey's HSD or Dunnett's test.

### Computer Graphics

OriginLab was used for data graphing unless indicated otherwise. Blender and ChemDraw were used to generate select graphics. Select graphics were created with BioRender.com.

### Example 5: In Vivo Studies of DFO-Treated Exosome-Loaded BIOGEL

In this study, presented is an innovative treatment strategy for TBI that hinges on the potential of bioengineered exosomes derived from human iPSC-derived NPCs, coupled with an advanced delivery system using an injectable BIOGEL. Exosomes were harvested from deferoxamine-treated human induced pluripotent stem cell (iPSC)-derived neuronal progenitor cells (NPCs) cultures. Deferoxamine, a hypoxia mimetic agent, was used to precondition the iPSCs, enhancing the regenerative capabilities of the derived NPCs under hypoxic conditions, a common sequelae of TBI. The innovation in this approach also lies in the encapsulation of these engineered exosomes within a bioorthogonal hydrogel by grafting gelatin with tetrazine and norbornene groups (GelTz-Nb, BIOGEL). Bioorthogonal chemistry, which enables reactions to occur inside living systems without interfering with native biochemical processes, provides a unique platform for drug delivery systems. BIOGEL is specifically designed to be injectable, allowing for minimally invasive administration. Once injected, the gel forms a matrix that provides a localized and sustained release of exosomes at the injury site. This targeted delivery is crucial in TBI treatment, where addressing the injury directly can significantly influence the therapeutic outcome.

The bioorthogonal nature of the gel ensures biocompatibility and minimizes immunogenic responses, which are often a concern with foreign substance administration into the body. This compatibility is vital for ensuring that the therapeutic potential of the exosomes is not hindered by adverse host reactions. Moreover, the gel's physical properties are fine-tuned to degrade over a controlled period, matching the therapeutic window required for effective TBI treatment.

In particular, this study involves: 1. characterization of engineered exosomes (detailing their biogenesis, molecular content, and the specific modifications induced by deferoxamine treatment), 2. evaluation of the interaction between these exosomes and the BIOGEL matrix, and 3. evaluation of the therapeutic efficacy of this system in ameliorating TBI. It was hypothesized that the synergy between NPC-derived exosomal therapy and the bioorthogonal gel delivery system represents a significant advancement in TBI treatment. By combining the intrinsic regenerative properties of NPC-derived exosomes with a targeted, sustained release system, this strategy holds promise for not only mitigating the immediate effects of TBI, but also for enhancing long-term neurological recovery.

### Results and Discussion

### DFO-Treated Exosome-Loaded BIOGEL Minimizes Scarring and Stabilizes Cells in the Cortex and Hippocampus

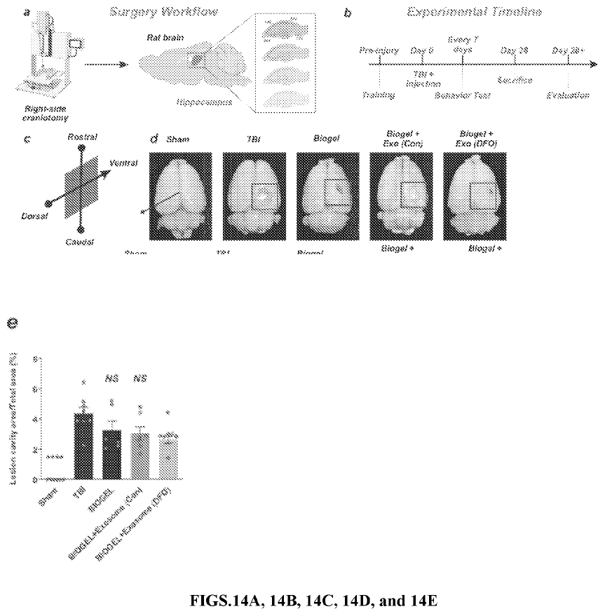

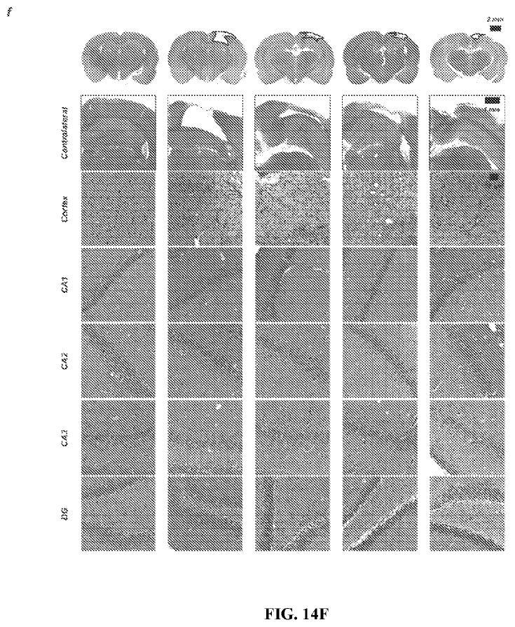

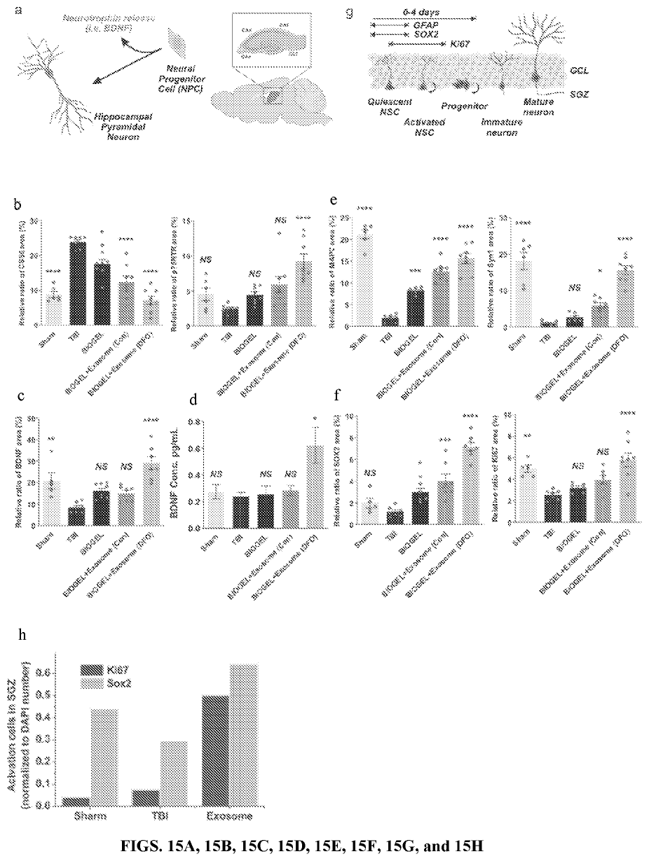

This study demonstrated a reduction in scarring from TBI and stabilization of altered cells in the cortex and hippocampus, as shown in FIGS. 14A-14F. The CCI model of brain injury was implemented by inducing trauma with an impactor between the bregma and lambda points on the rat brain. The brain samples were sectioned coronally, and the areas of interest, particularly the cortex and hippocampus, were examined due to their susceptibility to damage under the conditions of the experiment (FIG. 14A).

Prior to the TBI surgery, all animals underwent rotarod training for baseline behavioral assessment. Concurrently with the induction of TBI, therapeutic substances were administered. Behavioral tests were conducted at one-week intervals over a four-week period. Subsequently, on day 28 post-TBI, the animals were sacrificed, and samples of blood, CSF, and brain tissue were collected for comprehensive analysis (FIG. 14B).

A key focus of this in vivo experiment was to visually inspect the brain tissue for evidence of neuroregeneration following the implantation of materials such as BIOGEL+EXOSOME post-TBI. The 28-day post-injury mark was chosen for sample collection, aligning with the timeline of neurogenesis onset in animal models following TBI. Notably, the implantation of BIOGEL containing DFO exosomes appeared to significantly fill the voids at the lesion site in the cortex and reduce the scarring caused by the impactor (FIG. 14C).

Furthermore, stabilization in cellular changes was observed within the cortex and hippocampus. The traumatically injured brain tissue showed reduced scarring, and the cellular architecture in both the cortex and hippocampus areas appeared more stable.

To delve deeper into these findings, H&E staining was performed. This allowed for a closer examination of cellular changes and the precise location of lesions within the damaged cortex and hippocampus (FIGS. 14D-14F). The periphery of the cortex, encompassing the lesion site, exhibited distorted cell formations. TBI was observed to cause irregular cell arrangement and cell death. The hippocampal regions, including CA1, CA2, CA3, and the dentate gyrus (DG), also displayed irregularities in cellular layering (FIG. 14D). Quantitative analysis of the lesion cavity in the cortex, relative to the total brain area, indicated that the area was significantly reduced in samples treated with BIOGEL+Exosome (DFO) (FIG. 4F).

Following TBI, the lesion site in brain tissue is typically marked by alterations in the extracellular matrix, scar formation, and the accumulation of chondroitin sulfate proteoglycans (CSPGs) within the lesion core. These CSPGs are known to obstruct axonal growth, thereby impeding the repair of brain injury. This study aimed to investigate the effectiveness of exosomes, particularly those exposed to hypoxic conditions post-injury, in countering the negative impacts of CSPGs on neurogenesis. CSPG, notably neurotoxic at the core of the glial scar, is surrounded by reactive astrocytes. The inventors focused on these reactive astrocytes, which are known to both hinder and facilitate neuronal repair, to assess their role in the formation of glial scars. Additionally, the receptor p75NTR, belonging to the neurotrophin family, has a multifaceted role in promoting neuronal survival via TRK-mediated pathways and regulating neuronal cell death. Given that Brain-Derived Neurotrophic Factor (BDNF), a neuroprotective and restorative agent, can bind to both the p75NTR and the high-affinity tyrosine kinase (TrkB) receptors at elevated levels, the inventors concentrated on BDNF secretion as an indicator of neuroregeneration.

It was observed that the CSPG marker CS56 was minimally expressed in the dentate gyrus of the hippocampus, but more prominently in the cortex at the lesion site core. Remarkably, CS56 expression was significantly lower in samples treated with BIOGEL+Exosome (DFO) (FIG. 5B). In contrast, the expression level of p75NTR was observed in the cortex. The astrocyte marker GFAP exhibited high expression in the cortex and adjacent hippocampal areas, including the lesion site, particularly noted in the hippocampus on both sides post-TBI (FIG. 5C). Quantitative analysis showed lower expression in the treatment groups, excluding BIOGEL (FIG. 5D). Quantification of BDNF immunostaining in brain tissue was highest in the BIOGEL+Exosome (DFO) group (FIG. 5G). Furthermore, the ELISA quantitative results for BDNF concentration in CSF, obtained four weeks post-transplantation, indicated a significant increase. This data suggests that the use of hypoxia-induced exosomes in combination with biogels not only elevates BDNF levels, but also indicates that p75NTR, despite its low affinity for BDNF, contributes to neurotrophic signaling via TRK pathways.

### DFO-Treated Exosome-Loaded BIOGEL Promotes Early Brain Cell Growth and Connection Formation

In the study of neurogenesis, particularly following TBI, the dentate gyrus (DG), subgranular zone (SGZ), and subventricular zone (SVZ) of the hippocampus are critical, as they house the highest concentration of neural stem cells (NSCs) (FIG. 5E). Granule neurons in the DG, known to extend synapses and axons to the CA3 region, are directly linked with cognitive functions and neurological disorders. Consequently, the inventors focused on the DG and CA3 regions, examining the expression of Microtubule-associated protein 2 (Map2) and Synapsin1 (Syn1), which are crucial for dendritic specification and synaptogenesis, respectively.

In the DG region, the expression levels of Map2 and Syn1 in BIOGEL+Exosome (DFO) indicated slight variations. Notably, in the CA3 region, Map2 and Syn1 expressions were significantly reduced post-TBI, with a more pronounced difference in Syn1 expression at higher magnifications in the treatment group. Quantitative analysis in selected Regions of Interest (ROIs) of CA3 revealed significantly higher Map2 levels in BIOGEL+Exosome (DFO) compared to TBI (FIG. 5E). Similarly, Syn1 analysis showed a marked increase in BIOGEL+Exosome (DFO) as opposed to TBI.

The SGZ in the DG is known for initiating neurogenesis and proliferation of NSCs. To explore this, the inventors focused on the expression of Sox2, a marker for multipotent neural stem and progenitor cells, and Ki67, a nuclear protein indicative of the cell cycle phases, using immunofluorescence. Post-TBI, a decrease in Sox2 expression was observed in the hippocampus, particularly in the SGZ, when compared to other groups (FIG. 5H). Quantification of Sox2 through immunofluorescence revealed higher expression levels in BIOGEL+Exosome (DFO) (7.1967±1.025%) compared to the Sham group (1.988±0.852%) (FIG. 5F). Ki67 showed a similar trend, with higher expression levels in BIOGEL+Exosome (DFO). High magnification examination of Sox2 and Ki67 expressions in the SGZ highlighted a significantly enhanced level of activation in the BIOGEL+Exosome (DFO) group.

These findings suggest that the application of DFO-treated exosome-loaded BIOGEL facilitates early stages of neurogenesis and synaptogenesis, particularly in the critical neurogenic regions of the hippocampus.”

### DFO-Treated Exosome-Loaded BIOGEL Boosts Brain Remyelination After Injury

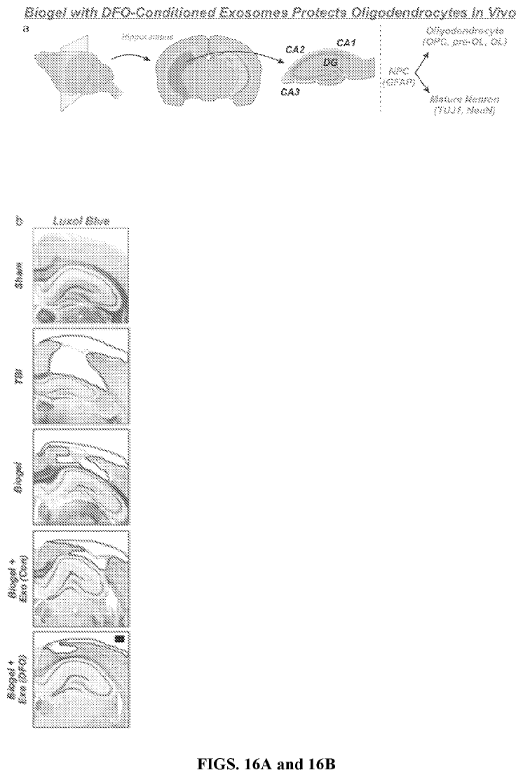

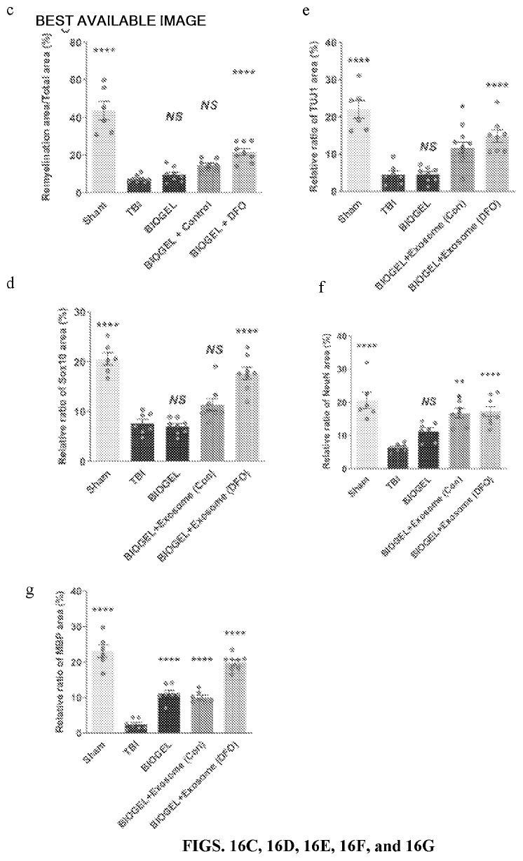

This study demonstrated a significant increase in the remyelination of oligodendrocytes in the brain following traumatic injury, achieved through the application of DFO-treated exosome-loaded BIOGEL. This was evidenced by the results of Luxol Fast Blue staining, which distinctly showed the most substantial remyelination effect in the group treated with DFO-treated exosome-loaded BIOGEL (FIGS. 16A and 16B). Further confirmation of this enhanced remyelination was obtained through immunofluorescence staining. Markers were utilized such as Sox 10, TUj1, NeuN, and MBP for this purpose. Across these markers, the results consistently indicated that the group receiving DFO-treated exosome-loaded BIOGEL exhibited superior remyelination effects (FIGS. 6C-6G).

These findings highlight the potential of DFO-treated exosome-loaded BIOGEL as a promising therapeutic approach for promoting remyelination in the context of brain injuries. The application of this novel treatment shows significant efficacy in facilitating the recovery and regeneration of oligodendrocytes, a crucial aspect of neural repair post-injury.”

### DFO-Treated Exosome-Loaded BIOGEL Improves Angiogenesis, Repairs Blood-Brain Barrier, Reduces Inflammation, and Boosts Functional Recovery

The application of DFO-treated exosome-loaded BIOGEL has demonstrated remarkable benefits in promoting angiogenesis, repairing the blood-brain barrier (BBB), reducing inflammatory responses, and facilitating functional recovery post brain injury. The repair of the BBB and the formation of new blood vessels are critical components of recovery following brain injury.

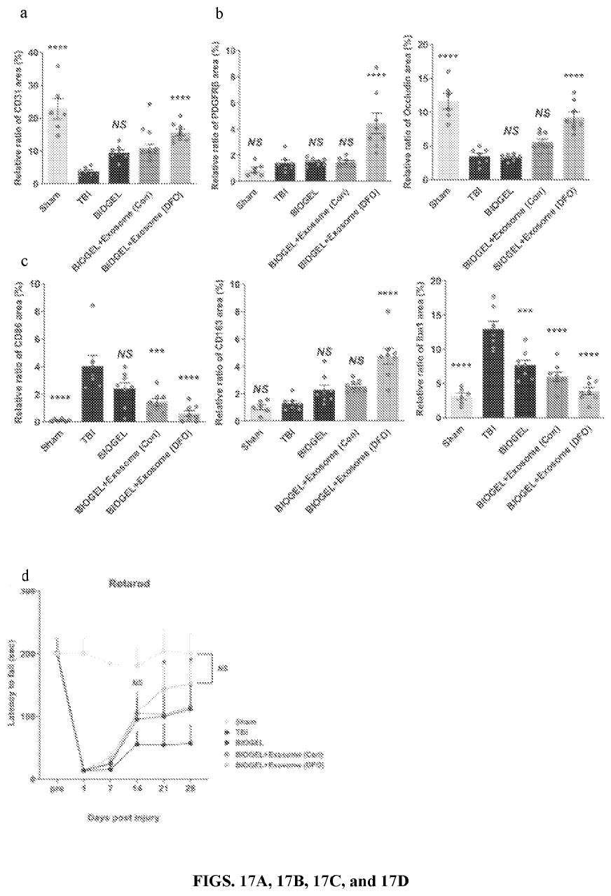

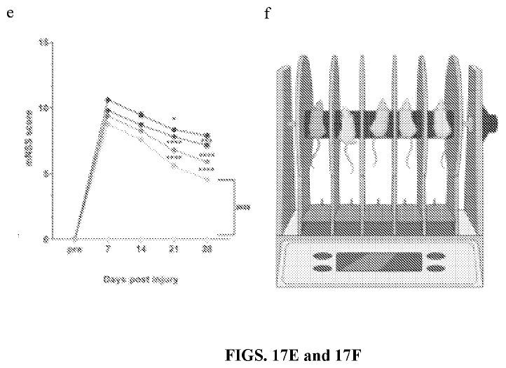

Through immunostaining with markers such as CD31, PDGFRβ, and occludin, this study confirmed that the group treated with DFO-treated exosome-loaded BIOGEL exhibited the most significant improvements in angiogenesis and BBB recovery (FIGS. 17A, 17B, and 17C). To further assess the impact on inflammatory responses post-injury, the IBA1 marker was employed, along with CD86 for M1 and CD163 for M2 polarization. Notably, the DFO-treated exosome-loaded BIOGEL group showed the greatest reduction in inflammation, as evidenced by the shift towards M2 polarization (FIGS. 17D, 17E, and 17F).

In addition to these molecular and cellular improvements, the functional recovery of the treated animals was evaluated through behavioral assessments, including the mNSS and the Rotarod test. These assessments were conducted over a four-week period following the treatment.

The results clearly indicated that the DFO-treated exosome-loaded BIOGEL group experienced the most substantial improvements in functional recovery (FIGS. 17D, 17E, and 17F).

Collectively, these findings underscore the potential of DFO-treated exosome-loaded BIOGEL as a comprehensive treatment strategy for brain injury, addressing key aspects of neurological repair and recovery.

### Experimental Methods

### Animal Model for Traumatic Brain Injury and Treatment

All procedures involving animals were conducted at the Laboratory Animal Center of CHA University of Science and Technology, in strict accordance with the guidelines approved by the Institutional Animal Care and Use Committee (IACUC-220189). Female Sprague-Dawley rats, aged 10 weeks (weighing approximately 230±30 g), were acquired from Koatech (Pyeongtaek, Korea) for these experiments. The animals were housed under controlled conditions: humidity (55%-65%), temperature (24° C.±3° C.), and a 12-hour light/dark cycle. They were acclimatized to these conditions for one week prior to any experimental procedures.

The surgical protocol was initiated with the administration of a combination of zolazepam and tiletamine (Zoletil, 50 mg/kg, intraperitoneal, Virbac Laboratories, France) and xylazine (Rompun, 10 mg/kg, intraperitoneal, Bayer, Korea) for anesthesia. Following anesthesia, the hair on the rat's skull was removed, and the exposed skin was disinfected using iodine and 70% ethanol. A precise incision was made in the skull skin, and a craniotomy was performed using a 1-mm round tip diamond burr (Strong 207A, SAESHIN PRECISION, Korea). The craniotomy site was carefully chosen to be a circular area of at least 5 mm diameter, situated between the Bregma and Lambda points of the skull.

For the induction of traumatic brain injury (TBI), a controlled cortical impact (CCI) was applied using a precision Impactor Device (#68099II, RWD life science, USA). The settings for the CCI were as follows: speed at 4.0 μm/s, depth at 3.0 mm, dwell time at 5.0 seconds, and a tip size of 5 (Ø, mm). This procedure resulted in moderate injury in severity. Post-injury, hemostasis was achieved, and various substances were administered into the brain tissue at the site of impact. The substances included BIOGEL alone, BIOGEL with control exosomes, and BIOGEL with Deferoxamine (DFO) conditioned exosomes. Following the injection, the incision was sutured, and the animals were placed on a heating pad to maintain their body temperature during recovery from anesthesia.

Postoperative care included the administration of Ketoprofen (1 mg/kg) and Cefazolin (15 mg/kg) in 0.9% Saline for three days. The experimental groups were divided as follows: (1) Sham (n=18), (2) TBI (n=18), (3) BIOGEL (n=15), (4) BIOGEL+iPSC-NPC derived Exosome (Control) (n=16), and (5) BIOGEL+iPSC-NPC derived Exosome (DFO-treated induced hypoxia) (n=16).

### Functional Recovery Assessments

The modified neurological severity score (mNSS) is a comprehensive behavioral analysis tool used to evaluate neurological function in animal models of brain injury. This scoring system encompasses assessments of sensory function, motor skills, reflexes, and balance. mNSS evaluations were conducted at multiple time points post-TBI—specifically on days 7, 14, 21, and 28. Each assessment was repeated three times to ensure consistency, adhering to established criteria and methodologies. The scoring range for mNSS classifies the severity of injury into mild (score 1-6), moderate (score 7-12), and severe (score 13-18). All assessments were performed by evaluators blinded to the experimental groups to maintain objectivity.

To assess the recovery of motor function after TBI, the Rotarod test was employed, which is a widely accepted method for evaluating motor coordination and balance in rodents. Prior to the surgical induction of TBI, all animals were acclimated to the Rotarod apparatus over a period of three days to standardize their performance baseline. The Rotarod test (Model #SK-RO02MR, SCITECH KOREA INC) was administered both pre-injury and on post-injury days 1, 7, 14, 21, and 28. During these tests, the Rotarod was set to gradually increase from a speed of 4 rpm to a maximum of 40 rpm, with each session lasting up to 300 seconds.

### Enzyme-Linked Immunosorbent Assay (ELISA)

Prior to euthanasia on day 28 post-TBI, samples of animal blood serum and cerebrospinal fluid (CSF) were meticulously collected for analysis. The CSF was extracted from the cisterna magna, a subarachnoid space at the base of the skull, filled with CSF. This procedure involved gently bending the animal's neck to expose the collection site, followed by careful insertion of a syringe to draw the clear CSF. The extracted CSF was then centrifuged at 3000 rpm at a temperature of 4° C. for 10 minutes. The processed samples were subsequently stored at −80° C. in a deep freezer for preservation.

Blood samples were collected from the dorsal aorta artery through a laparotomy procedure conducted prior to the perfusion of the animals. The collected blood was immediately placed in K2EDTA-coated tubes (#365974, BD Microtainer) to prevent coagulation. These samples were then centrifuged at 3000 rpm at room temperature (23° C.) for 15 minutes to separate the serum. Following centrifugation, the supernatant was carefully extracted and stored at −80° C. in a deep freezer.

For quantitative biochemical analysis, the CSF samples were assayed for Brain-Derived Neurotrophic Factor (BDNF) levels using the Total BDNF Quantikine ELISA Kit (#DBNT00, R&D Systems). Similarly, interleukin-10 (IL-10) levels in the blood serum were measured using the IL-10 ELISA Kit (#ab214566, Abcam). All analyses were conducted in strict accordance with the protocols provided by the respective manufacturers.

### Tissue Processing and Paraffin Sectioning for Histological Analysis

To conduct histological analysis and immunostaining, brain tissue samples were harvested from the animals on day 28 post-TBI. The perfusion process began with the flushing of over 100 mL of saline through the heart to clear the blood, followed by fixation of the entire body with 50 mL of 4% paraformaldehyde (#P2031, BIOSESANG, Korea). After the removal of the fixed rat skull, the brain was carefully extracted and submerged in 4% paraformaldehyde for 48 hours for further fixation.

Subsequently, the brain tissue underwent a graded dehydration process. This involved sequential immersion in increasing concentrations of ethanol—70%, 80%, 90%, 95%, and finally 99.9%—followed by a series of xylene solutions for clearing. The xylene solutions used were 50% xylene in 99.9% ethanol, 66.6% xylene in ethanol, and pure 99.9% xylene (#X0019, Samchun, Korea).

After dehydration and clearing, the tissue was embedded in fresh paraffin wax. This embedding process involved immersing the tissue in new paraffin twice, for an hour each time. The paraffin-embedded blocks were then sectioned using a microtome (#RM2255; Leica, Germany) to achieve a uniform thickness of 5 μm. The sections were rehydrated in 40% ethanol, warmed, and then floated in distilled water maintained at 48° C. to ensure proper expansion. Finally, the sections were carefully mounted on glass slides. All tissue sections were obtained in the coronal plane, ensuring that the lesion site was included in each sample.

### Luxol Fast Blue Staining (LFB)

Luxol Fast Blue staining was utilized to identify nerve cells and myelination in the brain tissue sections. The procedure was conducted on 5 μm thick sections prepared from paraffin-embedded animal brain tissues using a Luxol Fast Blue Staining Kit (#ab150675, Abcam, UK). Initially, the sections underwent deparaffinization in xylene (99.9%) and a graded series of ethanol (99.9%, 95%, 90%, 80%, 70%). Following deparaffinization, the sections were washed in distilled water (DW) and then stained with Luxol Fast Blue solution at a controlled room temperature of 25±2° C. for 24 hours.

After staining, the sections were washed in DW and differentiated in a 0.05% lithium carbonate solution for 20 seconds, followed by brief immersion in 70% alcohol. This step was succeeded by cytoplasmic staining with Cresyl echt violet for 3 minutes. The sections were then cleared twice in 99.9% xylene for 10 minutes each. Finally, the stained sections were mounted using Canada balsam (#D1242, Reagent Duksan, Korea) under a cover glass. Imaging of all LFB-stained slides was performed using a Zeiss Axio Scan Z1 digital slide scanner.

### Hematoxylin and Eosin Staining (H&E) for Tissue Morphology

For Hematoxylin and Eosin (H&E) staining, unstained slide samples were used. The slides were first deparaffinized in xylene (99.9%) for three 10-minute intervals, followed by rehydration through a descending ethanol series (99.9% twice for 5 minutes, then 95%, 90%, 80%, and 70% ethanol, each for 3 minutes). After rehydration, the slides were washed under running tap water for 3 minutes.

Nuclear staining was performed using Harris Hematoxylin (#3550, BBC Biochemical, USA) for 10 minutes, followed by a wash under running tap water for another 3 minutes. Decolorization was achieved by tapping in 1% Alcoholic HCl (1% HCl in 70% ethanol) three times, followed by a subsequent wash in running tap water for 3 minutes. Neutralization was carried out with 1% ammonia water (in DW) for 1 minute, followed by another wash in running tap water for 3 minutes. Cytoplasmic staining was then conducted using Eosin Y Alcoholic (#3610, BBC Biochemical, USA) for 1 minute.

The slides then underwent a dehydration process, first through an ascending ethanol series (70%, 80%, 90%, 95%) and then twice in 99.9% ethanol (for 1 minute and 3 minutes, respectively). This was followed by a clearing process in 99.9% xylene, performed twice for 10 minutes each. The final step involved mounting the slides using Canada balsam (#D1242, Reagent Duksan, Korea) under a cover glass. Imaging of all H&E-stained slides was similarly performed using the Zeiss Axio Scan Z1 digital slide scanner.

### Immunofluorescence (IF) Analysis

For immunofluorescence analysis, slide samples were first deparaffinized in a sequential manner using xylene (99.9%) and a graded series of ethanol (99.9%, 95%, 90%). Following this, the slides were washed with distilled water (DW) and treated with pepsin (#E06-50, GBI Labs, Bothell, WA, USA) for 10 minutes to facilitate antigen retrieval. Subsequently, the slides were washed twice with PBS-T (1×PBS (phosphate-buffered saline)+0.1% Tween 20 (#P9416-100 ML, Sigma-Aldrich), each wash lasting 2 minutes.

The next step involved blocking non-specific binding sites using a blocking solution (#E07-100, GBI Labs) for 1 hour. This was followed by incubation with the primary antibody, prepared in the appropriate dilution solution (#E09-300, GBI Labs), at 4° C. for 24 hours. Post-primary antibody incubation, the slides were washed twice for 2 minutes each with PBS-T.

For detection, the appropriate secondary antibody was applied for 50 minutes, considering the host specificity for each primary antibody used. After the secondary antibody incubation, the slides were washed three times with PBS-T, each for 2 minutes, and then rinsed with Dulbecco's PBS solution (DPBS, #SH30028.02, Cytiva, USA).

Counterstaining of the nuclei was performed using 300 mM DAPI (#D1306, Invitrogen) at a dilution of 1:1000 for 10 minutes. Following the counterstaining, the slides were washed once with PBS-T and then with DW. Mounting of the slides was completed using DAKO Mounting Medium (#S3023, Invitrogen). Imaging of the immunofluorescence-stained slides was conducted using a Zeiss Axio Scan Z1 digital slide scanner.

For quantitative analysis of the immunofluorescence images, Zen 3.1 Blue Edition software was utilized. To ensure objective and representative quantification, three random Regions of Interest (ROIs) were selected from each organization (tissue sample) for analysis.

### Statistical Analysis

The data acquired from the digital slide scanner (Zeiss Axio Scan Z1, Carl Zeiss) were analyzed using the Zeiss analysis software, Zen 3.1 Blue Edition. All the data was presented as the mean I standard error of the mean (SEM) to provide a clear and accurate representation of the findings.

For statistical analyses, a One-Way analysis of variance (ANOVA) was primarily employed, followed by Tukey's post-hoc test for multiple comparisons. This analysis was carried out using GraphPad Prism software (version 8.0.2, GraphPad Software). However, for the assessment of animal behavior, specifically mNSS scoring and Rotarod testing, a Two-Way ANOVA with Tukey's post-hoc test was utilized. This approach was chosen to more appropriately analyze the repeated measures and interactions between different factors in these behavioral tests.

Statistical significance in this study was determined based on p-values, with a threshold set at P<0.05 for statistical significance, indicating a 95% confidence interval. For highly significant results, a threshold of P<0.0001 was set, corresponding to a 99.9% confidence interval. Detailed information regarding the statistical significance and the specifics of the analysis can be found in the legends accompanying each figure in the results section.

### EQUIVALENTS

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A biogel comprising:
a gelatin polymer formed by crosslinking of a first gelatin precursor and a second gelatin precursor, wherein the first gelatin precursor comprises one or more tetrazine groups and the second gelatin precursor comprises one or more olefin groups; and
a biologically active material,
wherein the first gelatin precursor has a structure of Formula (I): 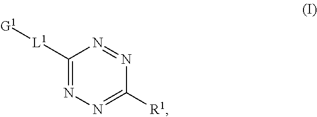
wherein:
G¹ is a gelatin protein, or a derivative or fragment thereof;
L¹ is a linking group selected from a bond, substituted or unsubstituted alkylene, or substituted or unsubstituted heteroalkylene; and
R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein the second gelatin precursor has a structure of Formula (II): 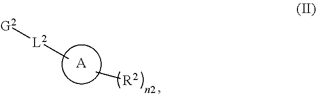
wherein:
G² is a gelatin protein, or a derivative or fragment thereof;
L² is a linking group selected from a bond, substituted or unsubstituted alkylene, or substituted or unsubstituted heteroalkylene;
Ring A is cycloalkenyl dienophile;
Each R² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein two or more R² may be same or different; or two or more R² may join to form a substituted or unsubstituted cycloalkyl, substituted or unsubstituted heterocycloalkyl, substituted or unsubstituted aryl, or substituted or unsubstituted heteroaryl; and
n2 is an integer from 0 to 6,
wherein G¹ and G² may be same or different.

2. The biogel composition of claim 1, wherein:
L¹ is —NH(CO)-L^{1A}-C(O)NH-L^{1B}-L^{1C}-, —NH(CO)-L^{1A}-C(O)-L^{1B}-L^{1C}-, or —NH(CO)-L^{1A}-NH-L^{1B}-L^{1C}-
L² is —NH(CO)-L^{2A}-C(O)NH-L^{2B}-L^{2C}-, —NH(CO)-L^{2A}-C(O)-L^{2B}-L^{2C}-, or —NH(CO)-L^{2A}-NH-L^{2B}-L^{1C}; and
Each L^{1A}, L^{1B}, L^{1C}, L^{2A}, L^{2B}, and L^{2C} is independently a bond, substituted or unsubstituted C₁-C₈ alkylene, or substituted or unsubstituted phenylene.

3. The biogel composition of claim 2, wherein each L¹ and L² is independently —NH(CO)—(CH₂)ₚ—C(O)NH—(CH₂)_{q}—, or —NH(CO)—(CH₂)ₚ—C(O)NH—(CH₂)_{q}—(C₆H₅)—.

4. The biogel composition of claim 1, wherein R¹ is hydrogen.

5. The biogel composition of claim 1, wherein n2 is 0 or 1.

6. The biogel composition of claim 1, wherein:
the first gelatin precursor has a structure of 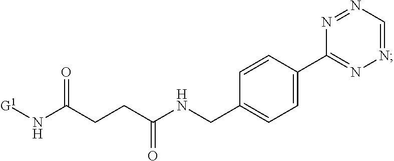
the second gelatin precursor has a structure of 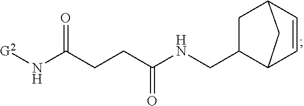
and
each G¹ and G² independently comprises a gelatin protein derived from aggrecan or collagen.

7. The biogel composition of claim 1, wherein the biologically active material comprises a protein, antibody, nucleic acid, biologic drug, peptide, small molecule, ligand, cytokine, chemotherapeutic agent, antipyretic, analgesic, anesthetic, antibiotic, antiseptic, hormone, stimulant, depressant, statin, beta blocker, anticoagulant, antiviral, anti-fungal, anti-inflammatory, growth factor, vaccine, cell, stem cell, tissue, diagnostic composition, psychiatric medication, psychoactive compound, or combinations thereof.

8. The biogel composition of claim 7, wherein the biologically active material comprises a growth factor.

9. The biogel composition of claim 8, wherein the biologically active material comprises TGF-β.

10. The biogel composition of claim 7, wherein the biologically active material comprises a stem cell.

11. The biogel composition of claim 1, further comprising one or more of manganese dioxide (MnO₂) nanosheets and/or a polymer.

12. The biogel composition of claim 1, wherein the biogel composition has a water content of about 10 to 80% by weight.

13. A method of treating a tissue injury in a subject comprising:
administering a biogel composition of claim 1 to the subject at the site of the tissue injury.

14. The method of claim 13, wherein the subject has been or is suffering from an injury to a neural tissue, a cartilage, a muscle, or a bone.

15. The method of claim 13, wherein the subject has been or is suffering from an injury to a neural tissue.

16. The method of claim 15, wherein the subject has been or is suffering from degenerated intervertebral discs (IVD) and the biogel is applied to the site of IVD degeneration.

17-22. (canceled)

23. A kit for treating a tissue in a subject, comprising:
a first vial comprising a first gelatin precursor;
a second vial comprising a second gelatin precursor;
a biologically active material, wherein the biologically active material is contained in the first vial and/or second vial; and
an applicator,
wherein the first gelatin precursor has a structure of Formula (I): 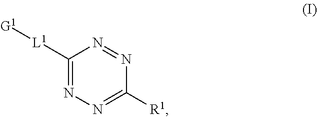
wherein:
G¹ is a gelatin protein, or a derivative or fragment thereof;
L¹ is a linking group selected from a bond, substituted or unsubstituted alkylene, or substituted or unsubstituted heteroalkylene; and
R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl,
wherein the second gelatin precursor has a structure of Formula (II): 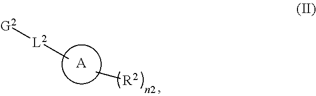
wherein:
G² is a gelatin protein, or a derivative or fragment thereof;
L² is a linking group selected from a bond, substituted or unsubstituted alkylene, or substituted or unsubstituted heteroalkylene;
Ring A is cycloalkenyl dienophile;
Each R² is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, wherein two or more R² may be same or different; or two or more R² may join to form a substituted or unsubstituted cycloalkyl, substituted or unsubstituted heterocycloalkyl, substituted or unsubstituted aryl, or substituted or unsubstituted heteroaryl; and
n2 is an integer from 0 to 6,
wherein G¹ and G² may be same or different.

24-34. (canceled)

35. A biocompatible tissue implant comprising the biogel of claim 1.

36. A tissue regenerating product comprising the biogel of claim 1.

37. An exosome comprising the biogel of claim 1.


SEQUENCE LISTING (30 sequences)

SEQ ID NO: 1 - DNA, 22 bp, Synthetic construct
  source 1..22: mol_type = other DNA
catgttccaatatgattccacc

SEQ ID NO: 2 - DNA, 21 bp, Synthetic construct
  source 1..21: mol_type = other DNA
gatgggatttccattgatgac

SEQ ID NO: 3 - DNA, 20 bp, Synthetic construct
  source 1..20: mol_type = other DNA
tcacagtgtgtggtcaacat

SEQ ID NO: 4 - DNA, 20 bp, Synthetic construct
  source 1..20: mol_type = other DNA
agcccctttgttctaagcca

SEQ ID NO: 5 - DNA, 23 bp, Synthetic construct
  source 1..23: mol_type = other DNA
aaacaacctgaaccttccaaaga

SEQ ID NO: 6 - DNA, 19 bp, Synthetic construct
  source 1..19: mol_type = other DNA
gcaagtctcattgaatcca

SEQ ID NO: 7 - DNA, 19 bp, Synthetic construct
  source 1..19: mol_type = other DNA
cccaaccctaaacatccaa

SEQ ID NO: 8 - DNA, 19 bp, Synthetic construct
  source 1..19: mol_type = other DNA
aaacagctccgcatcaacc

SEQ ID NO: 9 - DNA, 21 bp, Synthetic construct
  source 1..21: mol_type = other DNA
atgcagcaccattcaggtctg

SEQ ID NO: 10 - DNA, 20 bp, Synthetic construct
  source 1..20: mol_type = other DNA
ccagccgatgagtcacacag

SEQ ID NO: 11 - DNA, 21 bp, Synthetic construct
  source 1..21: mol_type = other DNA
cgacagcgaccagatcaagga

SEQ ID NO: 12 - DNA, 19 bp, Synthetic construct
  source 1..19: mol_type = other DNA
tgcattgcactcctttcat

SEQ ID NO: 13 - DNA, 19 bp, Synthetic construct
  source 1..19: mol_type = other DNA
tgccagagttcagtggtgg

SEQ ID NO: 14 - DNA, 19 bp, Synthetic construct
  source 1..19: mol_type = other DNA
cacaggcaaatcggccatc

SEQ ID NO: 15 - DNA, 21 bp, Synthetic construct
  source 1..21: mol_type = other DNA
gcggagtggaaacttttgtcc

SEQ ID NO: 16 - DNA, 23 bp, Synthetic construct
  source 1..23: mol_type = other DNA
gggaagcgtgtacttatccttct

SEQ ID NO: 17 - DNA, 19 bp, Synthetic construct
  source 1..19: mol_type = other DNA
ccctgaagtcgaggagctg

SEQ ID NO: 18 - DNA, 19 bp, Synthetic construct
  source 1..19: mol_type = other DNA
ctgctgcacctctaagcga

SEQ ID NO: 19 - DNA, 19 bp, Synthetic construct
  source 1..19: mol_type = other DNA
gccagcctcggaacaaaca

SEQ ID NO: 20 - DNA, 20 bp, Synthetic construct
  source 1..20: mol_type = other DNA
gctcagcgaatgaggaagga

SEQ ID NO: 21 - DNA, 19 bp, Synthetic construct
  source 1..19: mol_type = other DNA
cggagacgcatcacctctg

SEQ ID NO: 22 - DNA, 21 bp, Synthetic construct
  source 1..21: mol_type = other DNA
agggagtggaggagtcattcg

SEQ ID NO: 23 - DNA, 27 bp, Synthetic construct
  source 1..27: mol_type = other DNA
cttcaaatctcacagcagcagcacatc

SEQ ID NO: 24 - DNA, 21 bp, Synthetic construct
  source 1..21: mol_type = other DNA
ccacgggaaagacacaggtag

SEQ ID NO: 25 - DNA, 21 bp, Synthetic construct
  source 1..21: mol_type = other DNA
gctcttactgactggcatgag

SEQ ID NO: 26 - DNA, 20 bp, Synthetic construct
  source 1..20: mol_type = other DNA
cgcagctctaggagcatgtg

SEQ ID NO: 27 - DNA, 22 bp, Synthetic construct
  source 1..22: mol_type = other DNA
atcattgaccgctcctttaggt

SEQ ID NO: 28 - DNA, 19 bp, Synthetic construct
  source 1..19: mol_type = other DNA
gctcgccttgatggttcct

SEQ ID NO: 29 - DNA, 20 bp, Synthetic construct
  source 1..20: mol_type = other DNA
atgacatcaagaaggtggtg

SEQ ID NO: 30 - DNA, 20 bp, Synthetic construct
  source 1..20: mol_type = other DNA
cataccaggaaatgagcttg